(19) United States
(12) Patent Application Publication
(10) Pub. No.: US 2025/0228293 A1
(43) Pub. Date: Jul. 17, 2025

(54) AEROSOL PROVISION SYSTEM

(71) Applicant: NICOVENTURES TRADING LIMITED, London (GB)

(72) Inventors: Colin DICKENS, London (GB); Jeremy CAMPBELL, London (GB); Joanna SOFFE, London (GB); Dominic WOODCOCK, London (GB); Mark HARRIMAN, London (GB); Barry DIMMICK, London (GB); Thomas VICKERY, London (GB); Rosa GALATI, London (GB); David RUSHFORTH, London (GB); Andrew BURTON, London (GB); Theresa O'DONNELL, London (GB); Myles BATES, London (GB); Paul FROBISHER, London (GB); Trevor JOHNSON, London (GB)

(21) Appl. No.: 18/730,748

(22) PCT Filed: Jan. 19, 2023
(86) PCT No.: PCT/GB2023/050099

§ 371 Date: Jul. 19, 2024

(30) Foreign Application Priority Data
Jan. 21, 2022 (GB) 2200793.4

(51) Int. Cl. A24F 40/42; A24F 40/10; A24F 40/30

(52) U.S. Cl. CPC A24F 40/42; A24F 40/10; A24F 40/30

(57) ABSTRACT

An aerosol provision system (1) for generating an aerosol. The aerosol provision system comprises a first reservoir (31) for storing a first aerosol-generating material, wherein the aerosol provision system (1) is configured to generate a first aerosol using the first aerosol-generating material. The aerosol provision system (1) also comprises a second reservoir (101) for storing an active substance, or second aerosol-generating material such that the aerosol provision system (1) is configured to generate a second aerosol using the second aerosol-generating material. In this way, the first aerosol may be generated in a way which then allows the second aerosol, or active substance, to then be either added to, mixed with, and/or supplied alongside the first aerosol in a way which allows the user to effectively customise to what extent this first aerosol, which is delivered to the user, is supplemented with the second aerosol/active substance.

### FIELD

The present disclosure relates to aerosol provision systems such as, but not limited to, nicotine delivery systems (e.g. electronic cigarettes and the like).

### BACKGROUND

Electronic aerosol provision systems often employ an electronic cigarette (e-cigarette) or more generally an aerosol provision device. Such an aerosol provision system typically contains aerosolisable material (also called aerosol-generating material), such as a reservoir of fluid or liquid containing a formulation, typically but not necessarily including nicotine, or a solid material such as a tobacco-based product, from which a gas/vapour/aerosol is generated for inhalation by a user, for example through heat vaporisation. Thus, an aerosol provision system will typically comprise a vaporiser (also called an aerosol generator), e.g. a heating element, arranged to vaporise/aerosolise a portion of aerosolisable material to generate a vapour/aerosol.

Once a vapour has been generated, the vapour may be passed through flavouring material to add flavour to the vapour (for example, if the aerosolisable material was not itself flavoured), after which the (flavoured) vapour may be then delivered to a user via a mouthpiece from the aerosol provision system.

A potential drawback of existing aerosol provision systems and associated aerosol provision devices is of their relative inflexibility to control the aerosol which is delivered to the user during use. A further drawback is that certain flavouring materials, sensate materials and/or additives which are configured to be delivered to a user as an aerosol may be more effectively aerosolised at different environmental/temperature conditions than other flavouring materials and/or additives forming part of the delivered aerosol to the user.

Various approaches are therefore described herein which seek to help address or mitigate some of these issues, through the implementation of an aerosol provision system in which first and second reservoirs of differing aerosol generating materials may be provided, to allow a first aerosol to be generated from first aerosol generating material from the first reservoir, and to allow a second aerosol to be generated from second aerosol generating material from the second reservoir, and in some cases such that the aerosol provision system is configured to generate the first aerosol independently of the second aerosol. In this way, the first aerosol may be generated in a way which then allows the second aerosol to then be either added to, mixed with, and/or supplied alongside the first aerosol in a way which allows the user to effectively customise to what extent this first aerosol is supplemented with the second aerosol as part of an end aerosol which is delivered to the user (e.g. via a mouthpiece provided as part of the aerosol provision system).

In respect of the same, also described herein is a module, which can store the second aerosol generating material, and which can be retro-refitted to an existing aerosol provision system to allow the user to customise any delivered first aerosol from the existing aerosol provision system by either supplementing, or mixing, this first aerosol using a second aerosol generated from the second aerosol generating material in the module. Thus again in these embodiments, the first aerosol is generated independently of the second aerosol, such that the second aerosol is effectively an optional supplement to the existing aerosol generation properties of the aerosol provision system.

Also described herein is a module which may be used to couple to a container of fluid (such as a drinks bottle, e.g. a container comprising a shooter or shot), for providing a vapour/aerosol alongside, or mixed with, any fluid dispensed as part of the container.

Also described herein are aerosol provision systems in which various different upstream aerosol generating materials, e.g. comprising one or more acids, flavouring materials or other additives, may be aerosolised and delivered to a downstream first reservoir containing a different/first aerosol generating material, such to again allow the user to yet further customise how a first aerosol from this first aerosol generating material is ultimately supplied to the user via a mouthpiece of the aerosol provision system.

Also described herein are aerosol provision systems in which various different downstream aerosol generating materials, e.g. comprising different flavouring materials or other additives, may be aerosolised and delivered (potentially independently/selectively of each other) downstream of a first reservoir containing a different/first aerosol generating material, such to again allow the user to yet further customise how a first aerosol from this first aerosol generating material is ultimately supplied to the user alongside one or more (or even both, in some instances) of these downstream aerosol generating materials.

### SUMMARY

Thus according to a first aspect of certain embodiments there is provided an aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing a second aerosol-generating material, wherein the aerosol provision system is configured to generate a second aerosol using the second aerosol-generating material;
wherein the aerosol provision system is configured to generate the first aerosol independently of the second aerosol.

According to a second aspect of certain embodiments there is provided a consumable for use in an aerosol provision system for generating an aerosol, wherein the consumable comprises:
a first reservoir for storing a first aerosol-generating material, wherein the aerosol provision system is configured to generate a first aerosol using the first aerosol-generating material;
a second reservoir for storing a second aerosol-generating material, wherein the aerosol provision system is configured to generate a second aerosol using the second aerosol-generating material;
wherein the aerosol provision system is configured to generate the first aerosol independently of the second aerosol.

According to a third aspect of certain embodiments there is provided a consumable for use in an aerosol provision system for generating an aerosol, wherein the consumable comprises:
a first reservoir for storing a first aerosol-generating material, wherein the aerosol provision system is configured to generate a first aerosol using the first aerosol-generating material;
a second reservoir for storing a second aerosol-generating material, wherein the consumable is configured to generate a second aerosol using the second aerosol-generating material;
wherein the consumable is configured to generate the first aerosol independently of the second aerosol.

According to a fourth aspect of certain embodiments there is provided an aerosol provision system comprising a consumable according to the second or third aspects and an aerosol provision device, wherein the aerosol provision device comprises a consumable receiving section that includes an interface arranged to cooperatively engage with an interface from the consumable so as to releasably couple the consumable to the aerosol provision device.

According to a fifth aspect of certain embodiments there is provided a method of generating an aerosol in an aerosol provision system, wherein the method comprises:
generating a first aerosol using a first aerosol-generating material received from a first reservoir of the aerosol provision system;
generating a second aerosol using a second aerosol-generating material received from a second reservoir of the aerosol provision system;
wherein the first aerosol is generated independently of the second aerosol.

According to a sixth aspect of certain embodiments there is provided a method of retrofitting an aerosol provision system configured to generate a first aerosol using a first aerosol-generating material received from a first reservoir of the aerosol provision system, wherein the method comprises:
releasably coupling a module, comprising a second reservoir storing a second aerosol-generating material, to the aerosol provision system;
generating a first aerosol using the first aerosol-generating material;
generating a second aerosol, inside the module, using the second aerosol-generating material.

According to a seventh aspect of certain embodiments there is provided a module, for use in an aerosol provision system which is configured to generate a first aerosol using a first aerosol-generating material, wherein the module is configured to be releasably coupled to the aerosol provision system, and wherein the module comprises:
a second reservoir for storing a second aerosol-generating material; and
an attachment portion for releasably coupling the module to the aerosol provision system;
wherein the module is configured for receiving a first aerosol from the aerosol provision system when the module is coupled to the aerosol provision system using the attachment portion, and is configured to generate a second aerosol using the second aerosol-generating material when the module is receiving the first aerosol.

According to an eight aspect of certain embodiments there is provided an assembly comprising the module as described above, and the aerosol provision system to which the module is configured to be releasably coupled.

Other numerous aspects of certain embodiments are as defined in the claims and various clauses as recited at the end of this specification.

It will be appreciated that features and aspects of the invention described above in relation to the various aspects of the invention are equally applicable to, and may be combined with, embodiments of the invention according to other aspects of the invention as appropriate, and not just in the specific combinations described herein.

### BRIEF DESCRIPTION OF THE DRAWINGS

Embodiments of the invention will now be described, by way of example only, with reference to the accompanying drawings, in which:

FIG. 1 schematically represents in perspective view an aerosol provision system comprising a cartridge and aerosol provision device (shown separated) in accordance with certain embodiments of the disclosure;

FIG. 2 schematically represents in exploded perspective view of components of the cartridge of the aerosol provision system of FIG. 1;

FIGS. 3A to 3C schematically represent various cross-section views of a housing part of the cartridge of the aerosol provision system of FIG. 1;

FIGS. 4A and 4B schematically represent a perspective view and a plan view of a dividing wall element of the cartridge of the aerosol provision system of FIG. 1;

FIGS. 5A to 5C schematically represent two perspective views and a plan view of a resilient plug of the cartridge of the aerosol provision system of FIG. 1;

FIGS. 6A and 6B schematically represent a perspective view and a plan view of a bottom cap of the cartridge of the aerosol provision system of FIG. 1;

FIG. 7 schematically represents an embodiment of aerosol provision system in accordance with certain embodiments of the disclosure, and which employs a second reservoir for storing a second aerosol-generating material, such that the aerosol provision system is configured to generate a first aerosol independently of any generation of a second aerosol using the second aerosol-generating material.

FIG. 8A schematically represents an embodiment of aerosol provision system in accordance with certain embodiments of the disclosure, and which employs a second reservoir for storing a second aerosol-generating material, such that the aerosol provision system is configured to generate a first aerosol independently of any generation of a second aerosol using the second aerosol-generating material.

FIG. 8B schematically represents an embodiment of aerosol provision system in accordance with certain embodiments of the disclosure, and which employs a second reservoir for storing a second aerosol-generating material, such that the aerosol provision system is configured to generate a first aerosol independently of any generation of a second aerosol using the second aerosol-generating material.

FIG. 9 schematically represents embodiments of aerosol provision system (or assembly) in accordance with certain embodiments of the disclosure, and which employs a module, comprising a second reservoir storing a second aerosol-generating material, which is configured to releasably couple to the rest of the aerosol provision system, such as a consumable or mouthpiece therefrom.

FIG. 10 schematically represents embodiments of assemblies where a variant of module, similar to that from FIG. 9, may be fitted to a container of fluid, such as a drinks bottle, for providing an aerosol to the fluid from the container, in accordance with certain embodiments of the disclosure.

FIG. 11 schematically represents an embodiment of aerosol provision system in accordance with certain embodiments of the disclosure, and which employs multiple (primary/secondary) second reservoirs for storing potentially different (primary/secondary) second aerosol-generating materials, such that the aerosol provision system is configured to generate an aerosol using the first aerosol along with either of the primary/secondary second aerosol-generating materials. The same FIG. 11 also includes an optional third reservoir, which may be upstream of the first reservoir in some embodiments, for storing a third aerosol-generating material, in accordance with certain embodiments of the disclosure.

FIG. 12 schematically represents additional embodiments of aerosol provision system in accordance with certain embodiments of the disclosure, and which each employs a patch-like consumable for use with, and releasable engagement/attachment with, an aerosol provision system for generating a first aerosol, wherein the consumable comprises a reservoir for storing an active substance which is configured to be delivered to a user of the aerosol provision system, in accordance with certain embodiments of the disclosure.

FIG. 13 schematically represents an embodiment of aerosol provision system in accordance with certain embodiments of the disclosure, and which employs a sleeve-like consumable for use, which may be made of cardboard or paper for instance, and which is in releasable engagement/attachment with, an aerosol provision system for generating a first aerosol, wherein the consumable comprises a reservoir for storing an active substance which is configured to be delivered to a user of the aerosol provision system, in accordance with certain embodiments of the disclosure.

FIG. 14 schematically represents an embodiment of aerosol provision system in accordance with certain embodiments of the disclosure, and which employs a consumable for use with, and releasable engagement/attachment with, a mouthpiece from, an aerosol provision system for generating a first aerosol, wherein the consumable comprises a reservoir for storing an active substance which is configured to be delivered to a user of the aerosol provision system, in accordance with certain embodiments of the disclosure.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to non-combustible aerosol provision systems (such as an e-cigarette). According to the present disclosure, a “non-combustible” aerosol provision system is one where a constituent aerosolisable material of the aerosol provision system (or component thereof) is not combusted or burned in order to facilitate delivery to a user. Aerosolisable material, which also may be referred to herein as aerosol generating material or aerosol precursor material, is material that is capable of generating aerosol, for example when heated, irradiated or energized in any other way. The aerosolisable material may also be flavoured, in some embodiments.

Throughout the following description the term “e-cigarette” or “electronic cigarette” may sometimes be used, but it will be appreciated this term may be used interchangeably with an aerosol provision system. An electronic cigarette may also be known as a vaping device or electronic nicotine delivery system (END), although it is noted that the presence of nicotine in the aerosolisable material is not a requirement.

In some embodiments, the aerosol provision system is a hybrid device configured to generate aerosol using a combination of aerosolisable materials, one or a plurality of which may be heated. In some embodiments, the hybrid device comprises a liquid or gel aerosolisable material and a solid aerosolisable material. The solid aerosolisable material may comprise, for example, tobacco or a non-tobacco product.

Typically, the (non-combustible) aerosol provision system may comprise a cartridge/consumable part and a body/reusable/aerosol provision device part, which is configured to releasably engage with the cartridge/consumable part.

The aerosol provision system may be provided with a means for powering a vaporiser therein, and there may be provided an aerosolisable material transport element for receiving the aerosolisable material that is to be vaporised. The aerosol provision system may also be provided with a reservoir for containing aerosolisable material, and in some embodiments a further reservoir for containing flavouring material for flavouring a generated vapour from the aerosol provision system.

In some embodiments, the vaporiser may be a heater/heating element capable of interacting with the aerosolisable material so as to release one or more volatiles from the aerosolisable material to form a vapour/aerosol. In some embodiments, the vaporiser is capable of generating an aerosol from the aerosolisable material without heating. For example, the vaporiser may be capable of generating a vapour/aerosol from the aerosolisable material without applying heat thereto, for example via one or more of vibrational, mechanical, pressurisation or electrostatic means. In a further example, the vaporiser may be capable of generating a vapour/aerosol from the aerosolisable material without applying heat thereto by reducing pressure within a reservoir, for example, where air is drawn through an aerosol provision system a reduction in pressure may be observed across the second reservoir causing droplets or particles to be lifted off of a substrate (e.g. a porous substrate) comprised within the second reservoir, thereby generating a vapour/aerosol.

In some embodiments, the substance to be delivered may be an aerosolisable material which may comprise an active constituent, a carrier constituent and optionally one or more other functional constituents.

The active constituent may comprise one or more physiologically and/or olfactory active constituents which are included in the aerosolisable material in order to achieve a physiological and/or olfactory response in the user. The active constituent may for example be selected from nutraceuticals and nootropics. The active constituent may be naturally occurring or synthetically obtained. The active constituent may comprise for example nicotine, caffeine, taurine, theine, a vitamin such as B6 or B12 or C, melatonin, a cannabinoid, or a constituent, derivative, or combinations thereof. The active constituent may comprise a constituent, derivative or extract of tobacco or of another botanical. In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, or mixtures thereof.

We have found that by protonating nicotine present in aerosol generating materials and/or aerosols per se to varying degrees, such that the aerosol generating materials and the aerosol contains nicotine in unprotonated form and nicotine in protonated form, the final aerosol inhaled by a user provides desirable properties of flavour, impact, irritation, smoothness and/or nicotine reward for the user. We have particularly found that certain levels of acid addition are particularly favourable, namely wherein the total content of acid present in the solution is no greater than 5.0 mole equivalents based on the nicotine, preferably no greater than 2.5 mole equivalents based on the nicotine, more preferably no greater than 1.0 mole equivalents based on the nicotine, most preferably no greater than 0.6 mole equivalents based on the nicotine. At this level of acid addition aerosol generating materials may be provided having desirable properties of flavour, impact, irritation, smoothness and/or nicotine reward for the user both when the nicotine content is relatively low, such as 1.8 wt % nicotine or less and when the nicotine content is relatively high, such as greater than 1.8 wt % nicotine.

As discussed herein the aerosol generating materials and/or aerosols per se may comprise nicotine in unprotonated form and nicotine in protonated form. As will be understood by one skilled in the art, the protonated form of nicotine is prepared by reacting unprotonated nicotine with an acid. The acids are one or more suitable acids selected from lauric acid, myristic acid, salicylic acid, malic acid, citric acid, phosphoric acid, nicotinic acid, levulinic acid, tartaric acid, lactic acid, and/or a carbonic acid source, e.g. carbon dioxide. In some embodiments a single acid are be used to protonate nicotine. In some embodiments two or more acids are be used to protonate nicotine.

In some embodiments, a first aerosol-generating material comprises unprotonated nicotine and a second aerosol-generating material comprises one or more suitable acids selected from lauric acid, myristic acid, salicylic acid, malic acid, citric acid, phosphoric acid, nicotinic acid, levulinic acid, tartaric acid, lactic acid, and/or a carbonic acid source, e.g. carbon dioxide. In one aspect of this embodiment, the unprotonated nicotine is protonated in the aerosol-generating material by the second aerosol generated from the second aerosol-generating material. In another aspect of this embodiment, unprotonated nicotine is protonated in the first aerosol by the second aerosol generated from the second aerosol-generating material.

Nicotine may be provided at any suitable amount of the aerosol generating materials, such as any combination of the first aerosol-generating material; the second aerosol-generating material; and/or any further aerosol-generating materials (e.g. in a potential third reservoir), depending on the desired dosage when inhaled by the user. In some embodiments nicotine is present in an amount of no greater than 6 wt % based on the total weight of the aerosol generating materials. In some embodiments nicotine is present in an amount of from 0.4 to 6 wt % based on the total weight of the aerosol generating materials. In some embodiments nicotine is present in an amount of from 0.8 to 6 wt % based on the total weight of the aerosol generating materials. In some embodiments nicotine is present in an amount of from 1 to 6 wt % based on the total weight of the aerosol generating materials. In some embodiments nicotine is present in an amount of from 1.8 to 6 wt % based on the total weight of the aerosol generating materials.

In some embodiments, the active constituent is an olfactory active constituent and may be selected from a “flavour” and/or “flavourant” which, where local regulations permit, may be used to create a desired taste, aroma or other somatosensorial sensation in a product for adult consumers. In some instances such constituents may be referred to as flavours, flavourants, flavouring material, cooling agents, warming agents, and/or sweetening agents. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gasone or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

In some embodiments, the flavouring material (flavour) may comprise menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

The carrier constituent may comprise one or more constituents capable of forming an aerosol. In some embodiments, the carrier constituent may comprise one or more of water, glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate. The carrier constituent (or more generally the aerosol generating material) in accordance with some embodiments may comprise no more than 80% glycerine, or no more than 90% glycerine, and/or no more than to 10% water, and/or no more than to 20% water (each (% w/w)). For instance, potential more specific embodiments include carrier constituents (or more generally the aerosol generating material), which in some instances may be a liquid, comprising any of the following percentages (% w/w):

TABLE 1

36% glycerine; 54% propylene glycol, 10% water
50% glycerine; 50% propylene glycol
63% glycerine; 36% propylene glycol, 1% water
62% glycerine; 30% propylene glycol, 8% water
50% glycerine; 32% propylene glycol, 18% water
20% glycerine; 70% propylene glycol, 10% water
43.5% glycerine; 55% propylene glycol, 1.5% water

In potential related embodiments, the carrier constituent (or more generally the aerosol generating material) phrased more generally may comprise glycerol. In one embodiment, the carrier constituent (or more generally the aerosol generating material) may comprise at least 10% w/w glycerol. In one embodiment, the carrier constituent (or more generally the aerosol generating material) comprises at least 15% w/w glycerol. In one embodiment, the carrier constituent (or more generally the aerosol generating material) comprises at least 20% w/w glycerol. In one embodiment, the carrier constituent (or more generally the aerosol generating material) comprises at least 25% w/w glycerol. In one embodiment, the carrier constituent (or more generally the aerosol generating material) comprises at least 30% w/w glycerol.

In one embodiment, glycerol and propylene glycol are present in any individual of the herein described aerosol generating materials in the following amounts: 60 to 90% w/w propylene glycol; and 40 to 10% w/w glycerol, based on the total weight of glycerol and propylene glycol present in the material.

In one embodiment, glycerol and propylene glycol are present in any herein described aerosol generating material(s) in the following amounts: 70 to 80% w/w propylene glycol; and 30 to 20% w/w glycerol, based on the total weight of glycerol and propylene glycol present in the material.

In one embodiment, any herein described aerosol generating material(s) may comprise about 70% w/w propylene glycol and about 30% glycerol.

In one embodiment, any herein described aerosol generating material(s) may be a liquid at about 25° C.

The one or more other functional constituents may comprise one or more of pH regulators, colouring agents, preservatives, binders, fillers, stabilizers, and/or antioxidants.

### Cannabinoids

For completeness as well, and in accordance with some embodiments, any combination of the herein described aerosols or aerosolisable material/aerosol-generating material (such as any combination of the first aerosol-generating material; the second aerosol-generating material; and/or any further aerosol-generating materials (e.g. in a potential third reservoir, as will be described further in due course) may comprise one or more cannabinoids, and/or at least one cannabinoid compound/species—for instance any of the compound/species as noted as follows. In that respect, cannabinoids are a class of natural or synthetic chemical compounds that act on cannabinoid receptors (i.e., CB1 and CB2) in cells that repress neurotransmitter release in the brain. Cannabinoids are cyclic molecules exhibiting particular properties such as the ability to cross the blood-brain barrier with ease. Cannabinoids may be naturally occurring (phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), including its isomers Δ^{6a,10a}-tetrahydrocannabinol (Δ^{6a,10a}-THC), Δ^{6a(7)}-tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCV A).

Although the legal status of specific cannabinoids varies from jurisdiction to jurisdiction, certain cannabinoids, for example cannabidiol (CBD), tetrahydrocannabinol (THC) and cannabinol (CBN), are being considered for use in a wide variety of applications, such as in formulations for use in aerosol provision systems. However, the stability of cannabinoids, such as cannabidiol (CBD), tetrahydrocannabinol (THC) and cannabinol (CBN), has been found to vary depending on certain environmental conditions, such as exposure to air or light, or variation in temperature and pH. This may have unintended and detrimental consequences.

For example, CBD may oxidise and degrade when exposed to light and/or air to form cannabidiol hydroxyquinone (CBDHQ or HU-331) and its isomeric or functional derivatives. Furthermore, CBD may be converted to Δ⁹-tetrahydrocannabinol (Δ⁹-THC) in response to variations in temperature and/or pH. As a result, the accuracy of the specified cannabinoid content and/or concentration may vary widely in the formulations, while regulated and restricted cannabinoids may be produced unintentionally that will render the product as illicit or unlicensed in certain jurisdictions. As such, there is a desire to provide formulations comprising one or more cannabinoids that maintain a high degree of purity during manufacture and storage, and in turn prevent the loss or degradation of one or more cannabinoids, such as cannabidiol (CBD), tetrahydrocannabinol (THC) or cannabinol (CBN), in a formulation.

In some embodiments, any of the aerosol provision system comprises aerosol-generating materials that comprise at least one carboxylated cannabinoid, and wherein the system is configured to provide for selective decarboxylation of the carboxylated cannabinoid.

The carboxylated form of some cannabinoids may have a different stability profile compared to their decarboxylated form. For example, the carboxylated form of cannabidiol, cannabidiolic acid (CBDA), behaves differently in some solvent systems compared to the decarboxylated form (CBD). On the one hand, this difference in stability can be exploited since it is possible to deploy a particular form of the cannabinoid so as to achieve a desired stability profile. However, it is generally the case that cannabinoids exert a greater pharmacological effect in their decarboxylated form. Thus, providing a cannabinoid in its carboxylated form may be less desirable. It is, however, possible to convert cannabinoids from their carboxylated form to their decarboxylated form.

In some embodiment, the aerosol provision system provides for the selective decarboxylation of cannabinoids within the system. By “selective decarboxylation of the carboxylated cannabinoid” it is meant that the system is able to selectively increase the extent to which decarboxylation of the carboxylated cannabinoid takes place. This is advantageous, since it is possible to exploit the benefits of controlling the stability profile of the cannabinoid, whilst also allowing for the provision of an aerosol with a decarboxylate quantity similar to that which might be derived from an aerosol-generating material containing the decarboxylate form of the cannabinoid only.

During use, and due to the presence of a controller with variable power delivery to the aerosol generator (e.g. the heating element), it is possible for the user to operate the aerosol provision system so as to control the extent of in situ conversion of the carboxylated form to the decarboxylated form. Since the rate of in situ conversion for some cannabinoids will generally be dependent on temperature (see Cannabis and Cannabinoid Research. Volume 1.1, 2016, Decarboxylation Study of Acidic Cannabinoids: A Novel Approach Using Ultra-High-Performance Supercritical Fluid Chromatography/Photodiode Array-Mass Spectrometry) providing a higher power to an aerosol generator, e.g. a heater/heating element, will generally result in a higher localized temperature at the heater meaning that conversion from the carboxylated form to the decarboxylated form will generally be greater. As such, the user is able to control the system so as to provide an aerosol with varying amounts of decarboxylated cannabinoid. For example, where the carboxylated cannabinoid is CBDA, the user is able to control the system so as to provide an aerosol with varying amounts of CBD.

An alternative way the system may provide for the selective decarboxylation of the carboxylated cannabinoid is for the aerosol provision system to comprise either a first aerosol-generating material or a second aerosol-generating material, wherein either aerosol-generating material comprises the at least one carboxylated cannabinoid. For the avoidance of doubt, the use of this alternative approach can be combined with the other approaches described herein for the selective decarboxylation of the carboxylated cannabinoid.

Providing a carboxylated cannabinoid in either the first aerosol-generating material or the second aerosol-generating material can be beneficial for a number of reasons. Firstly, it can allow for the either aerosol-generating material to be subjected to selective heating to a temperature which is lower than the temperature to which the other aerosol-generating material is heated. For example, the second aerosol-generating material might comprise one or more carboxylated cannabinoid(s), which can be selectively heated (via power from a power source in the device or elsewhere) so as to facilitate decarboxylation of the carboxylated cannabinoid (e.g. CBD) contained therein. For the avoidance of doubt, the first aerosol-generating material may comprise one or more carboxylated cannabinoid(s), which can be selectively heated in the same manner. In some embodiments, one or more heating elements or heaters can be provided to heat the aerosol-generating materials. In some embodiments, the first reservoir or the second reservoir could contain an internal heating element which would be in contact with the aerosol-generating material contained therewithin and/or an external heater which would not be in contact with the aerosol-generating material. This heater configuration is not limited to systems comprising cannabinoids and can be used in connection with the other approaches described herein.

The extent to which the first reservoir or the second reservoir is heated affects the extent of decarboxylation that may occur. For example, the heating element (whether it be internal, external or both) may be configured to heat the second aerosol-generating material to a temperature above ambient, but below the temperature at which significant vaporization of the second aerosol-generating material would take place. In this regard, the second aerosol-generating material may be heated to a temperature such as greater than 50° C., greater than 60° C., greater than 70° C., greater than 80° C., greater than 90° C., greater than 100° C., greater than 110° C., greater than 120° C., greater than 130° C., greater than 140° C., or greater than 145° C. using an appropriate aerosol generator(s) 40 from the aerosol provision system 1 which may be configured to heat this second aerosol-generating material (e.g. using power from power source which is fed to this aerosol generator(s) 40 via the contact electrodes 46).

### Sensates

In some instances, any combination of the herein described aerosols or aerosolisable material/aerosol-generating material (such as any combination of the first aerosol-generating material; the second aerosol-generating material; and/or any further aerosol-generating materials (e.g. in a potential third reservoir) may comprise a flavouring material comprising one or more sensate or sensate compound (or combination(s) thereof). By the term “sensate compound” or “sensate”—used interchangeably herein—is meant a compound that triggers a sensation mediated by the trigeminal nerve of a user. The use of sensate compounds is well-documented in the food and pharmaceutical industry, and the triggered sensations include cooling, warming, and tingling sensations. When used in an aerosolisable material, such sensations should be experienced in the oral cavity, the nasal cavity and/or by the skin of the user. The present disclosure is not limited in this respect.

In one embodiment, the one or more sensates are selected from cooling agents, warming agents or tingling agents. The terms “cooling”, “warming” and “tingling” are well-understood in the art.

Cooling agents, warming agents and tingling agents are each typically small organic molecules which deliver a cooling, warming or tingling sensation to a user upon contact with the oral cavity, nasal cavity and/or skin. This sensation falls under the category of chemesthetic sensations and arises because the small organic molecule activates certain receptors in the skin and/or mucous membranes. The experience of a cooling, warming and/or tingling sensation thus relies on chemesthesis of the user. Chemesthesis is also referred to in the art as the “common chemical sense” or trigeminal chemosensation because it typically refers to sensations that are mediated by the trigeminal nerve and which are elements of the somatosensory system, distinguishing them from olfaction (sense of smell) and taste.

In one embodiment, the one or more sensates comprise a cooling agent. The cooling agent is typically not menthol. In one embodiment the one or more sensates comprise a cooling agent which is a compound of formula (I) or a salt and/or solvate thereof:

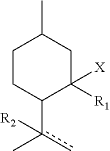

wherein X is hydrogen or OR′, wherein R′ is an alkyl group or an alkenyl group which may be taken together with R₁ to form a three to five-membered heterocyclyl group, wherein the heterocyclyl group is optionally substituted by one or more substituents selected from OH, O-alkyl, alkyl-OH, alkyl-O-alkyl, NH₂, NH-alkyl, N-(alkyl)₂, NO₂ and CN; and wherein R₁ and R₂ are each independently selected from hydrogen, OH, ORₐ, C(O)NR_{b}R_{c} and C(O)OR_{b}R_{c}; with the proviso that when R₁ is OH the compound of formula (I) is not menthol; and when the double bond is present, R₂ is absent;
wherein Rₐ is an alkyl group, an alkenyl group, a C(O)R_{f} group, or a C(O)-alkyl-C(O)R_{f} group wherein the alkyl groups and alkenyl groups are optionally substituted by one or more substituents selected from OH, O-alkyl, NH₂, NH-alkyl, N-(alkyl)₂, NO₂ and CN; and wherein R_{f} is an alkyl group, an alkenyl group, OH, O-alkyl, NH₂, NH-alkyl or N-(alkyl)₂, wherein the alkyl groups and alkenyl groups are optionally substituted by one or more substituents selected from OH, O-alkyl, NH₂, NH-alkyl, N-(alkyl)₂, NO₂ and CN;
wherein R_{b} and R_{c} are each independently hydrogen, an alkyl group, an alkenyl group, an aryl group, an aralkyl group, a heteroaryl group, or a heteroaralkyl group, wherein the alkyl groups, alkenyl groups, aryl groups and heteroaryl groups are optionally substituted by one or more substituents selected from OH, O-alkyl, NH₂, NH-alkyl, N-(alkyl)₂, NO₂, CN and C(O)R_{f}.

In one embodiment X is hydrogen.

In one embodiment X is OR′, wherein R′ is an alkyl group or an alkenyl group which is taken together with R₁ to form a three to five-membered heterocyclyl group, wherein the heterocyclyl group is optionally substituted by OH, O-alkyl or alkyl-OH. In one embodiment X is OR′, wherein R′ is an alkyl group which is taken together with R₁ to form a four or five-membered heterocyclyl group, wherein the heterocyclyl group is optionally substituted by alkyl-OH. In one embodiment X is OR′, wherein R′ is an alkyl group which is taken together with R₁ to form a four or five-membered heterocyclyl group, wherein the heterocyclyl group is optionally substituted by alkyl-OH, and wherein R₁ is ORₐ wherein Rₐ is an alkyl group and wherein R₂ is absent or hydrogen.

In one embodiment R₁ is selected from OH, ORₐ and C(O)NR_{b}R_{c} and R₂ is either absent or selected from OH and ORₐ. In one embodiment R₁ is OH with the proviso that the compound of formula (I) is not menthol. In one embodiment R₁ is OH and R₂ is selected from OH and ORₐ.

In one embodiment X is hydrogen and R₁ is selected from OH, ORₐ and C(O)NR_{b}R_{c}, with the proviso that, when R₁ is OH, the compound of formula (I) is not menthol. R₂ is either absent or selected from OH and ORₐ. In one embodiment X is hydrogen, R₁ is selected from ORₐ and C(O)NR_{b}R_{c} and R₂ is either absent or selected from OH and ORₐ.

In one embodiment R₁ is ORₐ and Rₐ is an alkyl group substituted by one or more OH substituents. R₂ may be hydrogen.

In one embodiment R₁ is ORₐ and Rₐ is a C(O)R_{f} group, or a C(O)-alkyl-C(O)R_{f} group, wherein R_{f} is an alkyl group optionally substituted by one or more OH substituents or R_{f} is OH. R₂ may be hydrogen.

In one embodiment R₁ is C(O)NR_{b}R_{c}, wherein R_{b} and R_{c} are each independently hydrogen, an alkyl group, an aryl group, an aralkyl group, a heteroaryl group, or a heteroaralkyl group. In one embodiment R₁ is C(O)NR_{b}R_{c} and at least one of R_{b} and R_{c} is hydrogen. R₂ may be hydrogen.

In one embodiment R₁ is C(O)NR_{b}R_{c}, wherein R_{b} is hydrogen and R_{c} is selected from the group consisting of an alkyl group, an aryl group, an aralkyl group and a heteroaralkyl group. R₂ may be hydrogen.

As used herein, the term “alkyl” includes both saturated straight chain and branched alkyl groups which may be substituted (mono- or poly-) or unsubstituted. In one embodiment the alkyl group is a C₁₋₁₀ alkyl group. In one embodiment the alkyl group is a C₁₋₈ alkyl group. In one embodiment the alkyl group is a C₁₋₆ alkyl group. In one embodiment the alkyl group is a C₁₋₃ alkyl group. In one embodiment the alkyl groups include, for example, methyl, ethyl, propyl, isopropyl, butyl, isobutyl, tert-butyl, pentyl and hexyl. In one embodiment the alkyl groups include methyl, ethyl, propyl or isopropyl.

As used herein, the term “alkenyl” includes both unsaturated straight chain and branched alkenyl groups which may be substituted (mono- or poly-) or unsubstituted. In one embodiment the alkenyl group is a C₂₋₁₀ alkenyl group. In one embodiment the alkenyl group is a C₂₋₈ alkenyl group. In one embodiment the alkenyl group is a C₂₋₆ alkenyl group. In one embodiment the alkenyl group is a C₂₋₃ alkenyl group.

As used herein, the term “aryl” refers to a C₆₋₁₂ aromatic group which may be substituted (mono- or poly-) or unsubstituted. Typical examples include phenyl and naphthyl etc. In one embodiment the aryl group is phenyl.

The term “aralkyl” is used as a conjunction of the terms alkyl and aryl as given above. For example an aryl group may be bonded to the compound of formula (I) through a diradical alkylene bridge, (—CH₂—)ₙ, where n is 1-10 and where “aryl” is as defined above. Alternatively an alkyl group may be bonded to the compound of formula (I) through a diradical aryl bridge, e.g. phenyl, where “alkyl is as defined above. In one embodiment the term “aralkyl” refers to a phenyl-alkyl group where the phenyl is bonded to the compound of formula (I).

As used herein the term “heteroaryl” refers to a monovalent aromatic group of from 1 to 12 carbon atoms having one or more oxygen, nitrogen, and sulfur heteroatoms within the ring. In one embodiment there are 1 to 4 oxygen, nitrogen and/or sulfur heteroatoms within the ring. In one embodiment there are 1 to 3 oxygen, nitrogen and/or sulfur heteroatoms within the ring. In one embodiment there are 2 oxygen and/or nitrogen heteroatoms within the ring. In one embodiment there is 1 oxygen or nitrogen heteroatom within the ring. The nitrogen and sulfur heteroatoms may optionally be oxidized. Such heteroaryl groups can have a single ring (e.g., pyridyl or furyl) or multiple condensed rings provided that the point of attachment is through a heteroaryl ring atom.

In one embodiment the heteroaryl is selected from the group consisting of pyridyl, pyridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, and benzothiophenyl. Heteroaryl rings may be unsubstituted or substituted. In one embodiment the heteroaryl is selected from the group consisting of pyridyl, pyridazinyl, pyrimidinyl, pyrazinyl, triazinyl and pyrrolyl. In one embodiment the heteroaryl is pyridyl.

As used herein the term “heterocyclyl” refers to fully saturated or unsaturated, monocyclic groups, which have one or more oxygen, sulfur or nitrogen heteroatoms in the ring. In one embodiment the heterocyclyl has 1 to 3 heteroatoms in the ring. In one embodiment the heterocyclyl has 1 to 3 oxygen and/or nitrogen heteroatoms in the ring. In one embodiment the heterocyclyl has 1 to 3 oxygen heteroatoms in the ring. The nitrogen and sulfur heteroatoms may optionally be oxidized and the nitrogen heteroatoms may optionally be quaternized. The heterocyclic group may be unsubstituted or substituted.

Exemplary monocyclic heterocyclic groups include, but are not limited to, pyrrolidinyl, pyrrolyl, pyrazolyl, oxiranyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, and triazinyl.

In one embodiment the heterocycyl is selected from the group consisting of oxiranyl, oxetanyl, tetrahydrofuryl, tetrahydropyranyl, and 1,3-dioxolane. In one embodiment the heterocycyl is 1,3-dioxolane.

All embodiments include, where appropriate, all enantiomers, tautomers and geometric isomers of the compounds of formula (I). The person skilled in the art will recognise compounds that possess optical properties (one or more chiral carbon atoms) or tautomeric characteristics. The corresponding enantiomers and/or tautomers may be isolated/prepared by methods known in the art. Some of the compounds of formula (I) may also exist as stereoisomers and/or geometric isomers—e.g. they may possess one or more asymmetric and/or geometric centres and so may exist in two or more stereoisomeric and/or geometric forms. All embodiments include, where appropriate, the use of all the individual stereoisomers and geometric isomers of those compounds, and mixtures thereof. The terms used in the claims encompass these forms.

Suitable salts of the compounds of formula (I) include suitable acid addition or base salts thereof. Such salts and solvates thereof will be known in the art. Suitable acid addition salts include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts.

In one embodiment, the one or more sensates comprise a cooling agent which is selected from the group consisting of:
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-2,3-trimethyl-2-propan-2-yl butanamide,
N-(2-pyridin-2-yl)ethyl) menthyl carboxamide,
menthone-1,2-glycerol ketal,
menthyl lactate,
isopulegol,
3-menthoxypropan-1,2-diol, and menthyl succinate.

In one embodiment the cooling agent is selected from the group consisting of:
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-2,3-trimethyl-2-propan-2-yl butanamide,
N-(2-pyridin-2-yl)ethyl) menthyl carboxamide,
menthone-1,2-glycerol ketal,
menthyl lactate,
3-menthoxypropan-1,2-diol, and
menthyl succinate.

In one embodiment the cooling agent may be selected from the group consisting of:
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-(2-pyridin-2-yl)ethyl) menthyl carboxamide,
menthone-1,2-glycerol ketal,
menthyl lactate,
isopulegol,
3-menthoxypropan-1,2-diol, and
menthyl succinate,
   or
   from the group consisting of:
N-ethyl-5-methyl-2-(propan-2-yl) cyclohexanecarboxamide,
ethyl-2-(5-methyl-2-propan-2-yl cyclohexanecarbonyl amino) acetate,
N-(4-methoxyphenyl)-p-menthanecarboxamide,
N-(2-pyridin-2-yl)ethyl) menthyl carboxamide,
menthone-1,2-glycerol ketal,
menthyl lactate,
3-menthoxypropan-1,2-diol, and
menthyl succinate.

In one embodiment, the cooling agent is selected from the group consisting of:

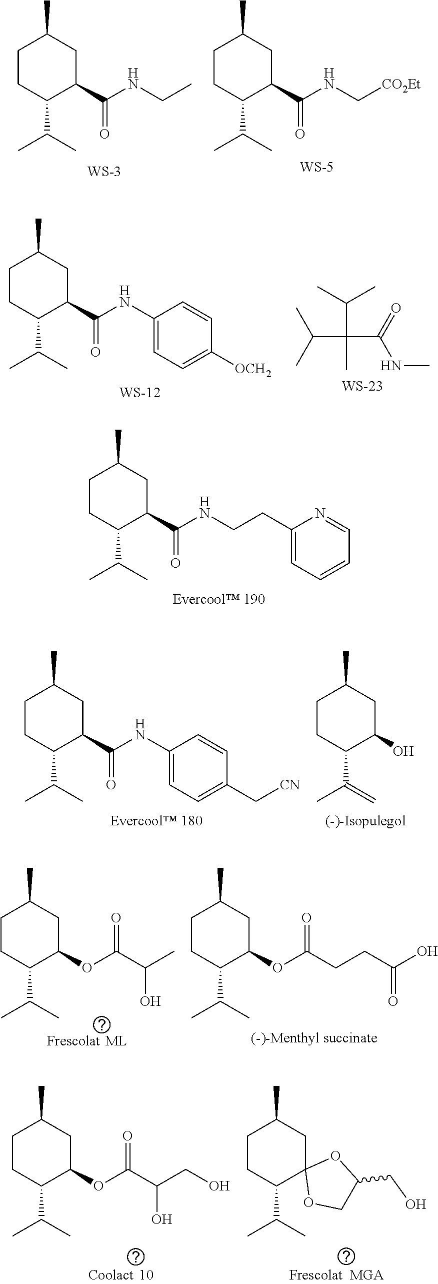

In one embodiment the cooling agent is not WS-23, i.e. N,2,3-trimethyl-2-propan-2-ylbutanamide.

In one embodiment the cooling agent is WS-23, i.e. N,2-3-trimethyl-2-propan-2-ylbutanamide.

In one embodiment the cooling agent is selected from the group consisting of (1S,2R,5S)—N-ethyl-5-methyl-2-(propan-2-yl) cyclohexanecarboxamide, ethyl-2-[[(1R,2S,5R)-5-methyl-2-propan-2-ylcyclohexanecarbonyl]amino] acetate, (1R,2S,5R)—N-(4-methoxyphenyl-p-menthanecarboxamide, (1R,2S,5R)—N-(2-(pyridin-2-yl)ethyl)menthylcarboxamide, (−)-menthone 1,2-glycerol ketal, (−)-menthyl lactate, (−)-isopulegol, 3-((−)-menthoxy)propane-1,2-diol, and (−)-menthyl succinate.

In one embodiment the cooling agent is selected from the group consisting of (1S,2R,5S)—N-ethyl-5-methyl-2-(propan-2-yl) cyclohexanecarboxamide, ethyl-2-[[(1R,2S,5R)-5-methyl-2-propan-2-ylcyclohexanecarbonyl]amino] acetate, (1R,2S,5R)—N-(4-methoxyphenyl-p-menthanecarboxamide, (1R,2S,5R)—N-(2-(pyridin-2-yl)ethyl) menthylcarboxamide, (−)-menthone 1,2-glycerol ketal, (−)-menthyl lactate, 3-((−)-menthoxy) propane-1,2-diol, and (−)-menthyl succinate.

In one embodiment the cooling agent is selected from the group consisting of (1S,2R,5S)—N-ethyl-5-methyl-2-(propan-2-yl) cyclohexanecarboxamide, ethyl-2-[[(1R,2S,5R)-5-methyl-2-propan-2-ylcyclohexanecarbonyl]amino] acetate, (1R,2S,5R)—N-(4-methoxyphenyl-p-menthanecarboxamide, (1R,2S,5R)—N-(2-(pyridin-2-yl)ethyl) menthylcarboxamide, (−)-menthone 1,2-glycerol ketal, (−)-menthyl lactate, (−)-isopulegol, and 3-((−)-menthoxy) propane-1,2-diol.

In one embodiment the cooling agent is selected from the group consisting of (1S,2R,5S)—N-ethyl-5-methyl-2-(propan-2-yl) cyclohexanecarboxamide, ethyl-2-[[(1R,2S,5R)-5-methyl-2-propan-2-ylcyclohexanecarbonyl]amino] acetate, ((1R,2S,5R)—N-(2-(pyridin-2-yl)ethyl) menthylcarboxamide, (−)-menthone 1,2-glycerol ketal, (−)-menthyl lactate, (−)-isopulegol, and 3-((−)-menthoxy) propane-1,2-diol.

In one embodiment the cooling agent is selected from the group consisting of (1S,2R,5S)—N-ethyl-5-methyl-2-(propan-2-yl) cyclohexanecarboxamide, ethyl-2-[[(1R,2S,5R)-5-methyl-2-propan-2-ylcyclohexanecarbonyl]amino] acetate, ((1R,2S,5R)—N-(2-(pyridin-2-yl)ethyl) menthylcarboxamide, (−)-menthone 1,2-glycerol ketal, (−)-menthyl lactate, and 3-((−)-menthoxy) propane-1,2-diol.

In one embodiment the cooling agent is (1R,2S,5R)—N-(2-(pyridin-2-yl)ethyl) menthylcarboxamide.

In another embodiment the cooling agent is (1S,2R,5S)—N-ethyl-5-methyl-2-(propan-2-yl) cyclohexanecarboxamide.

As noted above, all embodiments include, where appropriate, all enantiomers and tautomers of the compounds. All embodiments include, where appropriate, the use of all the individual stereoisomers and geometric isomers of those compounds, and mixtures thereof. The terms used in the claims encompass these forms.

In one embodiment, the one or more sensates comprise a warming agent or a tingling agent. In one embodiment, the warming agent or tingling agent is selected from the group consisting of vanilloids, sanshools, piperine, allyl isothiocyanate, cinnamyl phenylpropyl compounds, ethyl esters, and combinations thereof, or the warming agent or tingling agent is an extract from at least one of horseradish oil, ginger oil, black pepper, long pepper, Szechuan pepper, cayenne pepper, Uzazi or mustard oil.

Vanilloids are compounds which possess a vanillyl group, and a number of vanilloids bind to the transient receptor potential vanilloid type 1 or TRPV1 receptor, an ion channel which naturally responds to stimuli. TRPV1 is therefore an element of the mammalian somatosensory system. Vanilloids include capsaicin (8-methyl-N-vanillyl-6-nonenamide) and nonivamide as well as 3-phenylpropyl homovanillate, the major component of SymHeat PV used in the Examples herein. Other vanilloids include gingerols, zingerone, and shogaols as well as vanillyl ethyl ether, vanillyl propyl ether, vanillyl butyl ether and vanillyl butyl ether acetate.

Sanshools are exemplified by hydroxy-alpha-sanshool, a compound responsible for the numbing and tingling sensation caused by eating food cooked with Szechuan peppercorns and Uzazi. The term “sanshool” is derived from the Japanese term for the Japanese pepper and the suffix “ol” meaning “alcohol”. It is an agonist of TRPV1 and TRPA1 (an ion channel best known as a sensor for pain, cold, and itch in humans and other mammals).

Cinnamyl phenylpropyl compounds have a common structural characteristic of an aryl substituted primary alcohol/aldehyde/ester. They include 3-phenylpropyl cinnamate and 3-phenyl-1-propanol, which each have a spicy taste and balsamic odour, as well as 3-phenylpropyl isobutyrate which has a fruity taste and odour. In one embodiment, the cinnamyl phenylpropyl compounds are selected from 3-phenylpropyl cinnamate, 3-phenyl-1-propanol and combinations thereof.

In one embodiment, the warming agent or tingling agent is selected from the group consisting of vanilloids, sanshools, piperine, cinnamyl phenylpropyl compounds, ethyl esters, and combinations thereof, or the warming agent or tingling agent is an extract from at least one of ginger oil, black pepper, long pepper, Szechuan pepper, cayenne pepper, or Uzazi.

In one embodiment, the warming agent or tingling agent is selected from the group consisting of sanshools, allyl isothiocyanate, cinnamyl phenylpropyl compounds, ethyl esters, and combinations thereof, or the warming agent or tingling agent is an extract from at least one of horseradish oil, Szechuan pepper, cayenne pepper, Uzazi or mustard oil.

In one embodiment, the warming agent or tingling agent is selected from the group consisting of vanilloids, piperine, cinnamyl phenylpropyl compounds, ethyl esters, and combinations thereof, or the warming agent or tingling agent is an extract from at least one of ginger oil, black pepper, long pepper, or cayenne pepper.

In one embodiment, the warming agent or tingling agent comprises a combination of a vanilloid and a cinnamyl phenylpropyl compound. In one embodiment, the warming agent or tingling agent comprises a combination of a vanilloid and 3-phenylpropyl cinnamate, 3-phenyl-1-propanol, or a combination thereof. In one embodiment, the warming agent or tingling agent comprises a combination of a vanilloid and 3-phenylpropan-1-ol, such as 3-phenylpropyl homovanillate and 3-phenylpropan-1-ol.

In one embodiment, the warming agent or tingling agent is a combination of a vanilloid, an ethyl ester and a cinnamyl phenylpropyl compound. In one embodiment, the warming agent or tingling agent is a combination of a vanilloid, an ethyl ester, and 3-phenylpropyl cinnamate, 3-phenyl-1-propanol, or a combination thereof. In one embodiment, the warming agent or tingling agent comprises a combination of a vanilloid, an ethyl ester and 3-phenylpropan-1-ol, such as 3-phenylpropyl homovanillate and 3-phenylpropan-1-ol. The ethyl ester may, in any of these embodiments, be ethyl acetate.

In one embodiment, the warming agent or tingling agent is selected from the group consisting of vanillyl ethyl ether, vanillyl propyl ether, capsaicinoids, gingerols (e.g. [6], [8], [10] and/or [12]-gingerol), vanillyl butyl ether, vanillyl butyl ether acetate, sanshools, piperine, zingerone, shogaols (e.g. (6)-shogaol), allyl isothiocyanate, and combinations thereof, or the warming agent or tingling agent is an extract from at least one of horseradish oil, ginger oil, black pepper, long pepper, Szechuan pepper, cayenne pepper, Uzazi or mustard oil.

In one embodiment, the warming agent or tingling agent is selected from the group consisting of vanillyl ethyl ether, vanillyl propyl ether, vanillyl butyl ether, vanillyl butyl ether acetate, and combinations thereof.

In one embodiment, the warming agent or tingling agent is selected from the group consisting of capsaicinoids, gingerols (e.g. [6], [8], [10] and/or [12]-gingerol), sanshools, piperine, zingerone, shogaols (e.g. (6)-shogaol), allyl isothiocyanate, and combinations thereof, or the warming agent or tingling agent is an extract from at least one of horseradish oil, ginger oil, black pepper, long pepper, Szechuan pepper, cayenne pepper, Uzazi or mustard oil.

In one embodiment, the warming agent or tingling agent is selected from the group consisting of gingerols (e.g. [6], [8], [10] and/or [12]-gingerol), zingerone, shogaols (e.g. (6)-shogaol), and combinations thereof, or the warming agent or tingling agent is an extract from ginger oil.

In one embodiment, the warming agent or tingling agent is selected from the group consisting of vanillyl ethyl ether, capsaicinoids (e.g. capsaicin), gingerols, hydroxy-alpha-sanshool, piperine, zingerone, shogaols, allyl isothiocyanate, and combinations thereof, or the warming agent or tingling agent is an extract from at least one of horseradish oil, ginger oil, black pepper, long pepper, Szechuan pepper, cayenne pepper, Uzazi or mustard oil.

As noted above, all embodiments include, where appropriate, all enantiomers and tautomers of the compounds. The person skilled in the art will recognise compounds that possess optical properties (one or more chiral carbon atoms) or tautomeric characteristics. The corresponding enantiomers and/or tautomers may be isolated/prepared by methods known in the art.

Some of the compounds may also exist as stereoisomers and/or geometric isomers—e.g. they may possess one or more asymmetric and/or geometric centres and so may exist in two or more stereoisomeric and/or geometric forms. All embodiments include, where appropriate, the use of all the individual stereoisomers and geometric isomers of those compounds, and mixtures thereof. The terms used in the claims encompass these forms. Piperine has, for example, four geometric isomers including chavicine, isochavicine and isopiperine. The term “piperine” is used herein to refer to all the individual geometric isomers, and mixtures thereof.

In one embodiment the one or more sensates consist of cooling agents.

In one embodiment the one or more sensates consist of warming agents.

In one embodiment the one or more sensates consist of tingling agents.

In one embodiment the one or more sensates consist of cooling agents and warming agents.

In one embodiment the one or more sensates consist of cooling agents and tingling agents.

In one embodiment the one or more sensates consist of warming agents and tingling agents.

The above definitions of cooling agents, warming agents and tingling agents apply to each of these embodiments. For example, the one or more sensates may consist of cooling agents wherein the cooling agents are compounds of formula (I) or a salt and/or solvate thereof. In one embodiment the one or more sensates are cooling agents selected from the group consisting of a compound of formula (I) or a salt and/or solvate thereof or N,2,3-trimethyl-2-propan-2-ylbutanamide. Alternatively, as an example, the one or more sensates may consist of warming agents or tingling agents as defined above. In one embodiment the one or more sensate is a warming agent selected from the group consisting of hydroxy-alpha sanshool, capsaicin, piperine, zingerone, gingerol, a shogaol, allyl isothiocyanate and combinations thereof, or an extract from horseradish oil, ginger oil, black pepper, long pepper, Szechuan pepper, cayenne pepper, mustard oil or Uzazi. In one embodiment the one or more sensate is a tingling agent which is a combination of a vanilloid such as 3-phenylpropyl homovanillate, an ethyl ester such as ethyl acetate, and 3-phenylpropan-1-ol.

The aerosolisable material includes the one or more sensates in an amount of 0.01 to 12% w/w. This concentration range and the concentrations defined below apply to the above definitions of the one or more sensates. For example, the concentration range of 0.01 to 10% w/w applies to the one or more sensates comprising a cooling agent, a warming agent, a tingling agent or a combination thereof. The concentration range of 0.01 to 10% w/w also applies to the one or more sensates with a narrower definition, e.g. consisting of cooling agents, warming agents or tingling agents, etc. The range of 0.01 to 10% w/w is being used here as an example, the present disclosure is not limited to the combination of this concentration range with the specified sensate.

Furthermore, the sensate concentrations are combinable with the above-defined concentrations of the at least one cannabinoid.

In one embodiment the one or more sensate as defined herein is present in the material in an amount of from about 0.01% w/w to about 12% w/w. In one embodiment the one or more sensate as defined herein is present in the material in an amount of from about 0.05% w/w to about 12% w/w. In one embodiment the one or more sensate as defined herein is present in the material in an amount of from about 0.1% w/w to about 12% w/w.

In another embodiment, the one or more sensate as defined herein is present in the material in an amount of no greater than about 10% w/w. In one embodiment the one or more sensate as defined herein is present in the material in an amount of from about 0.01% w/w to about 10% w/w. In one embodiment the one or more sensate as defined herein is present in the material in an amount of from about 0.05% w/w to about 10% w/w. In one embodiment the one or more sensate as defined herein is present in the material in an amount of from about 0.1% w/w to about 10% w/w.

In another embodiment the one or more sensate as defined herein is present in the material in an amount of no greater than about 8% w/w. In one embodiment the one or more sensate as defined herein is present in the material in an amount of from about 0.01% w/w to about 8% w/w. In one embodiment the one or more sensate as defined herein is present in the material in an amount of from about 0.05% w/w to about 8% w/w. In one embodiment the one or more sensate as defined herein is present in the material in an amount of from about 0.1% w/w to about 8% w/w.

In another embodiment the one or more sensate as defined herein is present in the material in an amount of no greater than about 5% w/w. In one embodiment the one or more sensate as defined herein is present in the material in an amount of from about 0.01% w/w to about 5% w/w. In one embodiment the one or more sensate as defined herein is present in the material in an amount of from about 0.05% w/w to about 5% w/w. In one embodiment the one or more sensate as defined herein is present in the material in an amount of from about 0.1% w/w to about 5% w/w.

In another embodiment the one or more sensate as defined herein is present in an amount of no greater than about 3% w/w, e.g. no greater than about 2.5% w/w. In one embodiment the one or more sensate as defined herein is present in the material in an amount of from about 0.01% w/w to about 2.5% w/w. In one embodiment the one or more sensate as defined herein is present in the material in an amount of from about 0.05% w/w to about 2.5% w/w. In one embodiment the one or more sensate as defined herein is present in the material in an amount of from about 0.1% w/w to about 2.5% w/w.

In some embodiments, the one or more sensate and its concentration is selected based on its solubility in a propylene glycol/glycerol system. For example, the aerosolisable material may include an amount of the one or more sensate in a carrier constituent comprising at least 50% propylene glycol, and glycerol, where the carrier constituent is present at 70% w/w or more of the aerosolisable material, such that the aerosolisable material has a turbidity of ≤1.0 NTU. Turbidity and its measurement is discussed further herein.

Where any sensate or sensate compound is employed for any combination(s) of aerosol(s) or aerosol-generating material(s) herein described, it will be appreciated that these may be employed with or without a flavouring material as part of the same aerosol(s) or aerosol-generating material(s) and/or as part of a different aerosol(s) or aerosol-generating material(s) from the aerosol provision system.

In some embodiments, the aerosolisable material comprises a terpene. In some embodiments, the terpene is a terpene derivable from a phytocannabinoid producing plant, such as a plant from the strain of the Cannabis sativa species, such as hemp. In some embodiments, the aerosolisable material comprises a cannabinoid isolate in combination with a terpene derivable from a phytocannabinoid producing plant.

Suitable terpenes in this regard include so-called “C10” terpenes, which are those terpenes comprising 10 carbon atoms. Further, suitable terpenes in this regard also include so-called “C15” terpenes, which are those terpenes comprising 15 carbon atoms. In some embodiments, the aerosolisable material comprises more than one terpene. For example, the aerosolisable material may comprise one, two, three, four, five, six, seven, eight, nine, ten or more terpenes as defined herein.

In some embodiments, any given herein described aerosol generating material (such as a first aerosol generating material, and/or a second aerosol generating material) may comprise a combination of terpenes. In some embodiments, the combination of terpenes may comprise a combination of at least geraniol and linalool. In some embodiments, the combination of terpenes may comprise a combination of at least eucalyptol and menthone. In some embodiments, the combination of terpenes may comprise a combination of at least eucalyptol, carvone, piperitone and menthone. In some embodiments, the combination of terpenes may comprise a combination of at least eucalyptol, carvone, beta-bourbonene, germacrene, piperitone, iso-menthone and menthone.

For completeness therefore, it may be appreciated that any given herein described aerosol generating material may typically contain a variety of components that are to be delivered to a user. Depending on the mode of action of the aerosol generator for a given aerosol generating material from the aerosol provision system, these components for the related aerosol generating material may be influenced by the aerosol generator in different ways.

### EXAMPLES

As noted above, aerosol provision systems (e-cigarettes) may often comprise a modular assembly including both a reusable part (body—or aerosol provision device) and a replaceable consumable (cartridge) part. Devices conforming to this type of two-part modular configuration may generally be referred to as two-part devices. It is also common for electronic cigarettes to have a generally elongate shape. For the sake of providing a concrete example, certain embodiments of the disclosure described herein may comprise this kind of generally elongate two-part device employing consumable parts. However, it will be appreciated the underlying principles described herein may equally be adopted for other electronic cigarette configurations, for example modular devices comprising more than two parts, as devices conforming to other overall shapes, for example based on so-called box-mod high performance devices that typically have a more boxy shape.

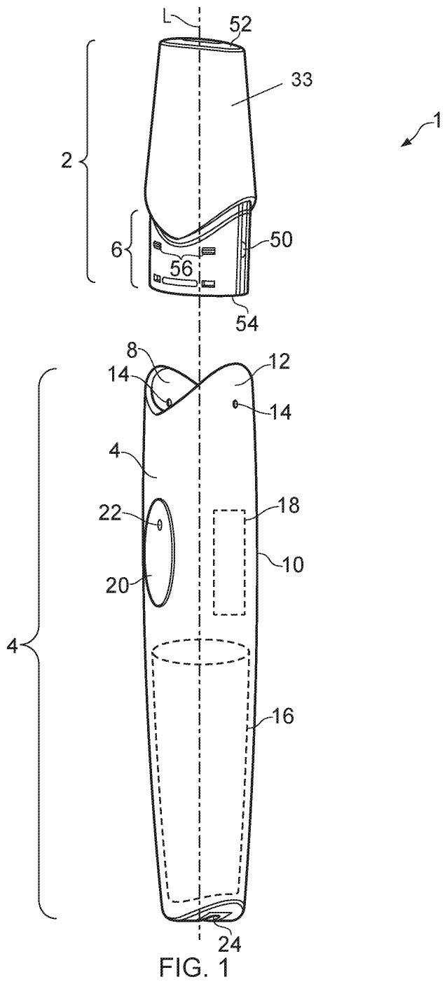

From the forgoing therefore, and with reference to FIG. 1 is a schematic perspective view of an example aerosol provision system (e-cigarette) 1 in accordance with certain embodiments of the disclosure. Terms concerning the relative location of various aspects of the electronic cigarette (e.g. terms such as upper, lower, above, below, top, bottom etc.) are used herein with reference to the orientation of the electronic cigarette as shown in FIG. 1 (unless the context indicates otherwise). However, it will be appreciated this is purely for ease of explanation and is not intended to indicate there is any required orientation for the electronic cigarette in use.

The e-cigarette 1 (aerosol provision system 1) comprises two main components, namely a cartridge 2 and an aerosol provision device 4. The aerosol provision device 4 and the cartridge 2 are shown separated in FIG. 1, but are coupled together when in use.

The cartridge 2 and aerosol provision device 4 are coupled by establishing a mechanical and electrical connection between them. The specific manner in which the mechanical and electrical connection is established is not of primary significance to the principles described herein and may be established in accordance with conventional techniques, for example based around a screw thread, bayonet, latched or friction-fit mechanical fixing with appropriately arranged electrical contacts/electrodes for establishing the electrical connection between the two parts as appropriate. For example electronic cigarette 1 represented in FIG. 1, the cartridge comprises a mouthpiece 33, a mouthpiece end 52 and an interface end 54 and is coupled to the aerosol provision device by inserting an interface end portion 6 at the interface end of the cartridge into a corresponding receptacle 8/receiving section of the aerosol provision device. The interface end portion 6 of the cartridge is a close fit to be receptacle 8 and includes protrusions 56 which engage with corresponding detents in the interior surface of a receptacle wall 12 defining the receptacle 8 to provide a releasable mechanical engagement between the cartridge and the aerosol provision device. An electrical connection is established between the aerosol provision device and the cartridge via a pair of electrical contacts on the bottom of the cartridge (not shown in FIG. 1) and corresponding sprung contact pins in the base of the receptacle 8 (not shown in FIG. 1). As noted above, the specific manner in which the electrical connection is established is not significant to the principles described herein, and indeed some implementations might not have an electrical connection between the cartridge and an aerosol provision device at all, for example because the transfer of electrical power from the reusable part to the cartridge may be wireless (e.g. based on electromagnetic induction techniques).

The electronic cigarette 1 (aerosol provision system) has a generally elongate shape extending along a longitudinal axis L. When the cartridge is coupled to the aerosol provision device, the overall length of the electronic cigarette in this example (along the longitudinal axis) is around 12.5 cm. The overall length of the aerosol provision device is around 9 cm and the overall length of the cartridge is around 5 cm (i.e. there is around 1.5 cm of overlap between the interface end portion 6 of the cartridge and the receptacle 8 of the aerosol provision device when they are coupled together). The electronic cigarette has a cross-section which is generally oval and which is largest around the middle of the electronic cigarette and tapers in a curved manner towards the ends. The cross-section around the middle of the electronic cigarette has a width of around 2.5 cm and a thickness of around 1.7 cm. The end of the cartridge has a width of around 2 cm and a thickness of around 0.6 mm, whereas the other end of the electronic cigarette has a width of around 2 cm and a thickness of around 1.2 cm. The outer housing of the electronic cigarette is in this example is formed from plastic. It will be appreciated the specific size and shape of the electronic cigarette and the material from which it is made is not of primary significance to the principles described herein and may be different in different implementations. That is to say, the principles described herein may equally be adopted for electronic cigarettes having different sizes, shapes and/or materials.

The aerosol provision device 4 may in accordance with certain embodiments of the disclosure be broadly conventional in terms of its functionality and general construction techniques. In the example of FIG. 1, the aerosol provision device 4 comprises a plastic outer housing 10 including the receptacle wall 12 that defines the receptacle 8 for receiving the end of the cartridge as noted above. The outer housing 10 of the aerosol provision device 4 in this example has a generally oval cross section conforming to the shape and size of the cartridge 2 at their interface to provide a smooth transition between the two parts. The receptacle 8 and the end portion 6 of the cartridge 2 are symmetric when rotated through 180° so the cartridge can be inserted into the aerosol provision device in two different orientations. The receptacle wall 12 includes two aerosol provision device air inlet openings 14 (i.e. holes in the wall). These openings 14 are positioned to align with an air inlet 50 for the cartridge when the cartridge is coupled to the aerosol provision device. A different one of the openings 14 aligns with the air inlet 50 of the cartridge in the different orientations. It will be appreciated some implementations may not have any degree of rotational symmetry such that the cartridge is couplable to the aerosol provision device in only one orientation while other implementations may have a higher degree of rotational symmetry such that the cartridge is couplable to the aerosol provision device in more orientations.

The aerosol provision device further comprises a battery 16 for providing operating power for the electronic cigarette, control circuitry 18 for controlling and monitoring the operation of the electronic cigarette, a user input button 20, an indicator light 22, and a charging port 24.

The battery 16 in this example is rechargeable and may be of a conventional type, for example of the kind normally used in electronic cigarettes and other applications requiring provision of relatively high currents over relatively short periods. The battery 16 may be recharged through the charging port 24, which may, for example, comprise a USB connector.

The input button 20 in this example is a conventional mechanical button, for example comprising a sprung mounted component which may be pressed by a user to establish an electrical contact in underlying circuitry. In this regard, the input button may be considered an input device for detecting user input, e.g. to trigger aerosol generation, and the specific manner in which the button is implemented is not significant. For example, other forms of mechanical button or touch-sensitive button (e.g. based on capacitive or optical sensing techniques) may be used in other implementations, or there may be no button and the device may rely on a puff detector for triggering aerosol generation.

The indicator light 22 is provided to give a user with a visual indication of various characteristics associated with the electronic cigarette, for example, an indication of an operating state (e.g. on/off/standby), and other characteristics, such as battery life or fault conditions. Different characteristics may, for example, be indicated through different colours and/or different flash sequences in accordance with generally conventional techniques.

The control circuitry 18 is suitably configured/programmed to control the operation of the electronic cigarette to provide conventional operating functions in line with the established techniques for controlling electronic cigarettes. The control circuitry (processor circuitry) 18 may be considered to logically comprise various sub-units/circuitry elements associated with different aspects of the electronic cigarette's operation. For example, depending on the functionality provided in different implementations, the control circuitry 18 may comprises power supply control circuitry for controlling the supply of power from the battery/power supply to the cartridge in response to user input, user programming circuitry for establishing configuration settings (e.g. user-defined power settings) in response to user input, as well as other functional units/circuitry associated functionality in accordance with the principles described herein and conventional operating aspects of electronic cigarettes, such as indicator light display driving circuitry and user input detection circuitry. It will be appreciated the functionality of the control circuitry 18 can be provided in various different ways, for example using one or more suitably programmed programmable computer(s) and/or one or more suitably configured application-specific integrated circuit(s)/circuitry/chip(s)/chipset(s) configured to provide the desired functionality.

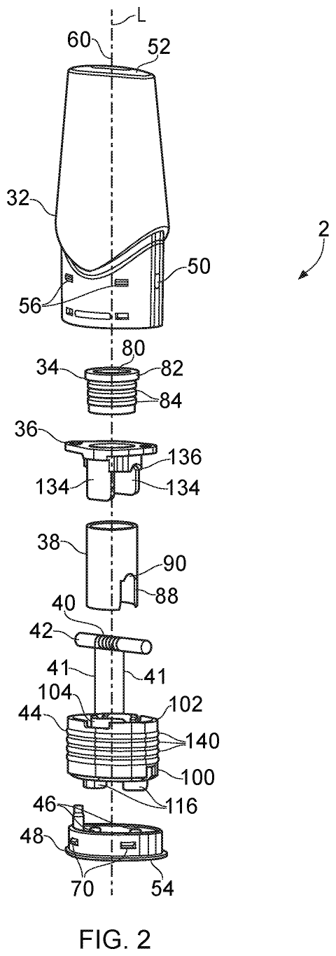

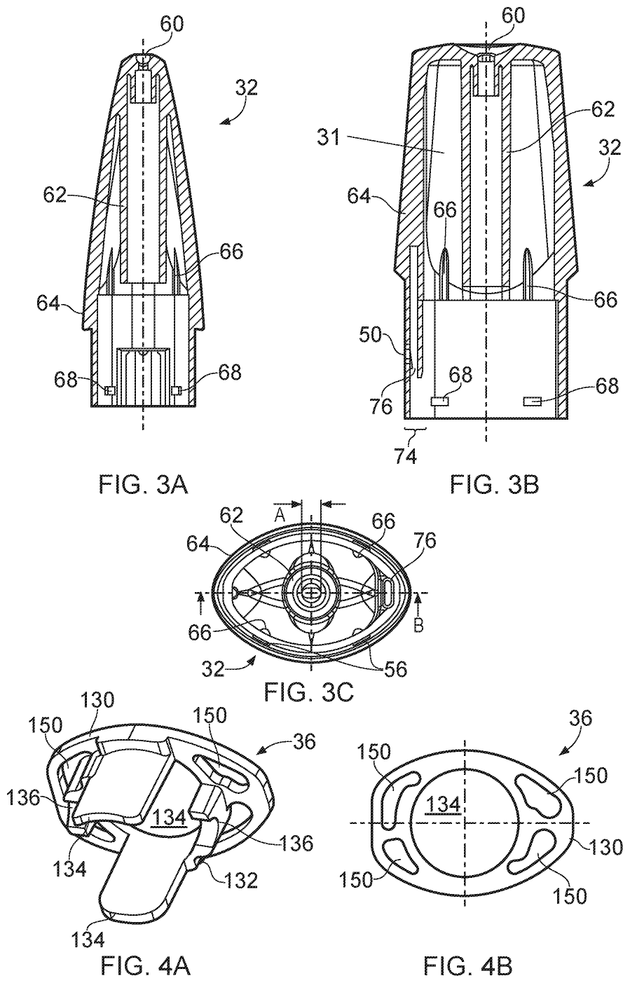

FIG. 2 is an exploded schematic perspective view of the cartridge 2 (exploded along the longitudinal axis L). The cartridge 2 comprises a housing part 32, an air channel seal 34, a dividing wall element 36, an outlet tube 38, a vaporiser/heating element 40, an aerosolisable material transport element 42, a plug 44, and an end cap 48 with contact electrodes 46. FIGS. 3 to 6 schematically represents some of these components in more detail.

FIG. 3A is a schematic cut-away view of the housing part 32 through the longitudinal axis L where the housing part 32 is thinnest. FIG. 3B is a schematic cut-away view of the housing part 32 through the longitudinal axis L where the housing part 32 is widest. FIG. 3C is a schematic view of the housing part along the longitudinal axis L from the interface end 54 (i.e. viewed from below in the orientation of FIGS. 3A and 3B).

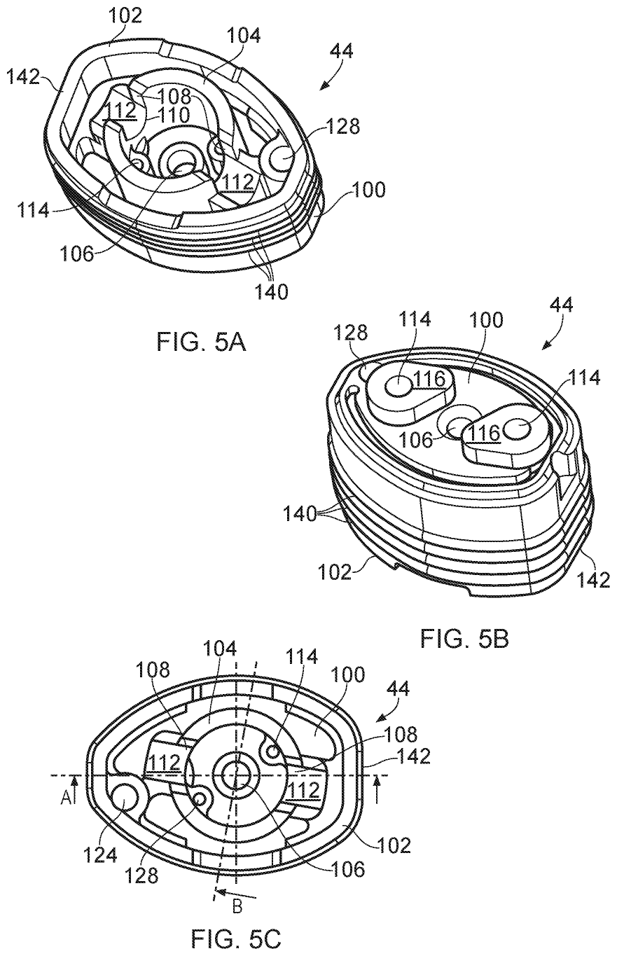

FIG. 4A is a schematic perspective view of the dividing wall element 36 as seen from below. FIG. 4B is a schematic cross-section through an upper part of the dividing wall element 36 as viewed from below.

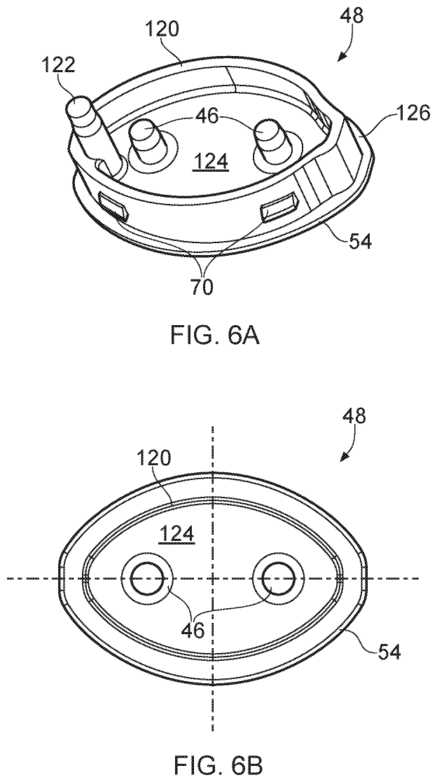

FIG. 5A is a schematic perspective view of the plug 44 from above and FIG. 5B is a schematic perspective view of the plug 44 from below. FIG. 5C is a schematic view of the plug 44 along the longitudinal axis L seen from the mouthpiece end 52 of the cartridge (i.e. viewed from above for the orientation in FIGS. 1 and 2).

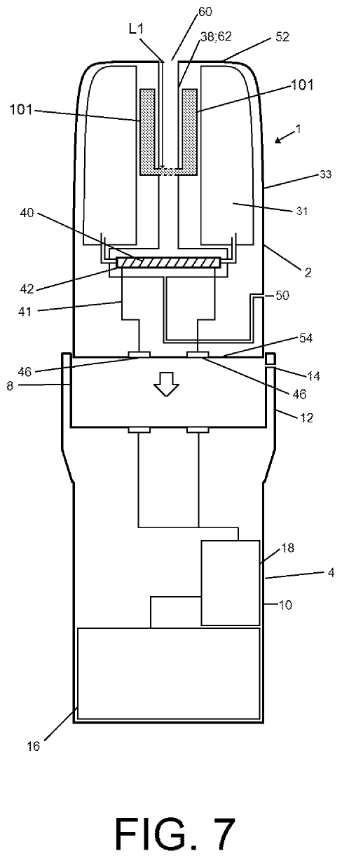

FIG. 6A is a schematic perspective view of the end cap 48 from above. FIG. 6B is a schematic view of the end cap 48 along the longitudinal axis L seen from the mouthpiece end 52 of the cartridge (i.e. from above).

The housing part 32 in this example comprises a housing outer wall 64 and a housing inner tube 62 which in this example are formed from a single moulding of polypropylene. The housing outer wall 64 defines the external appearance of the cartridge 2 and the housing inner tube 62 defines a part the air channel through the cartridge. The housing part is open at the interface end 54 of the cartridge and closed at the mouthpiece end 52 of the cartridge except for a mouthpiece opening/aerosol outlet 60, from the mouthpiece 33, which is in fluid communication with the housing inner tube 62. The housing part 32 includes an opening in a sidewall which provides the air inlet 50 for the cartridge. The air inlet 50 in this example has an area of around 2 mm². The outer surface of the outer wall 64 of the housing part 32 includes the protrusions 56 discussed above which engage with corresponding detents in the interior surface of the receptacle wall 12 defining the receptacle 8 to provide a releasable mechanical engagement between the cartridge and the aerosol provision device. The inner surface of the outer wall 64 of the housing part includes further protrusions 66 which act to provide an abutment stop for locating the dividing wall element 36 along the longitudinal axis L when the cartridge is assembled. The outer wall 64 of the housing part 32 further comprises holes which provide latch recesses 68 arranged to receive corresponding latch projections 70 in the end cap to fix the end cap to be housing part when the cartridge is assembled.

The outer wall 64 of the housing part 32 includes a double-walled section 74 that defines a gap 76 in fluid communication with the air inlet 50. The gap 76 provides a portion of the air channel through the cartridge. In this example the doubled-walled section 74 of the housing part 32 is arranged so the gap defines an air channel running within the housing outer wall 64 parallel to the longitudinal axis with a cross-section in a plane perpendicular to the longitudinal axis of around 3 mm². The gap/portion of air channel 76 defined by the double-walled section of the housing part extends down to the open end of the housing part 32.

The air channel seal 34 is a silicone moulding generally in the form of a tube having a through hole 80. The outer wall of the air channel seal 34 includes circumferential ridges 84 and an upper collar 82. The inner wall of the air channel seal 34 also includes circumferential ridges, but these are not visible in FIG. 2. When the cartridge is assembled the air channel seal 34 is mounted to the housing inner tube 62 with an end of the housing inner tube 62 extending partly into the through hole 80 of the air channel seal 34. The through hole 80 in the air channel seal has a diameter of around 5.8 mm in its relaxed state whereas the end of the housing inner tube 62 has a diameter of around 6.2 mm so that a seal is formed when the air channel seal 34 is stretched to accommodate the housing inner tube 62. This seal is facilitated by the ridges on the inner surface of the air channel seal 34.

The outlet tube 38 comprises a tubular section, for instance made of ANSI 304 stainless steel or polypropylene, with an internal diameter of around 8.6 mm and a wall thickness of around 0.2 mm. The bottom end of the outlet tube 38 includes a pair of diametrically opposing slots 88 with an end of each slot having a semi-circular recess 90. When the cartridge is assembled the outlet tube 38 mounts to the outer surface of the air channel seal 34. The outer diameter of the air channel seal is around 9.0 mm in its relaxed state so that a seal is formed when the air channel seal 34 is compressed to fit inside the outlet tube 38. This seal is facilitated by the ridges 84 on the outer surface of the air channel seal 34. The collar 80 on the air channel seal 34 provides a stop for the outlet tube 38.

The aerosolisable material transport element 42 comprises a capillary wick and the vaporiser (aerosol generator) 40 comprises a resistance wire heater wound around the capillary wick. In addition to the portion of the resistance wire wound around the capillary wick, the vaporiser comprises electrical leads 41 which pass through holes in the plug 44 to contact electrodes 46 mounted to the end cap 54 to allow power to be supplied to the vaporiser via the electrical interface the established when the cartridge is connected to an aerosol provision device. The vaporiser leads 41 may comprise the same material as the resistance wire wound around the capillary wick, or may comprise a different material (e.g. lower-resistance material) connected to the resistance wire wound around the capillary wick. In this example the heater coil 40 comprises a nickel iron alloy wire and the wick 42 comprises a glass fibre bundle. The vaporiser and aerosolisable material transport element may be provided in accordance with any conventional techniques and is may comprise different forms and/or different materials. For example, in some implementations the wick may comprise fibrous or solid a ceramic material and the heater may comprise a different alloy. In other examples the heater and wick may be combined, for example in the form of a porous and a resistive material. More generally, it will be appreciated the specific nature aerosolisable material transport element and vaporiser is not of primary significance to the principles described herein.

When the cartridge is assembled, the wick 42 is received in the semi-circular recesses 90 of the outlet tube 38 so that a central portion of the wick about which the heating coil is would is inside the outlet tube while end portions of the wick are outside the outlet tube 38.

The plug 44 in this example comprises a single moulding of silicone, may be resilient. The plug comprises a base part 100 with an outer wall 102 extending upwardly therefrom (i.e. towards the mouthpiece end of the cartridge). The plug further comprises an inner wall 104 extending upwardly from the base part 100 and surrounding a through hole 106 through the base part 100.

The outer wall 102 of the plug 44 conforms to an inner surface of the housing part 32 so that when the cartridge is assembled the plug in 44 forms a seal with the housing part 32. The inner wall 104 of the plug 44 conforms to an inner surface of the outlet tube 38 so that when the cartridge is assembled the plug 44 also forms a seal with the outlet tube 38. The inner wall 104 includes a pair of diametrically opposing slots 108 with the end of each slot having a semi-circular recess 110. Extended outwardly (i.e. in a direction away from the longitudinal axis of the cartridge) from the bottom of each slot in the inner wall 104 is a cradle section 112 shaped to receive a section of the aerosolisable material transport element 42 when the cartridge is assembled. The slots 108 and semi-circular recesses 110 provided by the inner wall of the plug 44 and the slots 88 and semi-circular recesses 90 of the outlet tube 38 are aligned so that the slots 88 in the outlet tube 38 accommodate respective ones of the cradles 112 with the respective semi-circular recesses in the outlet tube and plug cooperating to define holes through which the aerosolisable material transport element passes. The size of the holes provided by the semi-circular recesses through which the aerosolisable material transport element passes correspond closely to the size and shape of the aerosolisable material transport element, but are slightly smaller so a degree of compression is provided by the resilience of the plug 44. This allows aerosolisable material to be transported along the aerosolisable material transport element by capillary action while restricting the extent to which aerosolisable material which is not transported by capillary action can pass through the openings. As noted above, the plug 44 includes further openings 114 in the base part 100 through which the contact leads 41 for the vaporiser pass when the cartridge is assembled. The bottom of the base part of the plug includes spacers 116 which maintain an offset between the remaining surface of the bottom of the base part and the end cap 48. These spacers 116 include the openings 114 through which the electrical contact leads 41 for the vaporiser pass.

The end cap 48 comprises a polypropylene moulding with a pair of gold-plated copper electrode posts 46 mounted therein.

The ends of the electrode posts 44 on the bottom side of the end cap are close to flush with the interface end 54 of the cartridge provided by the end cap 48. These are the parts of the electrodes to which correspondingly aligned sprung contacts in the aerosol provision device 4 connect when the cartridge 2 is assembled and connected to the aerosol provision device 4. The ends of the electrode posts on the inside of the cartridge extend away from the end cap 48 and into the holes 114 in the plug 44 through which the contact leads 41 pass. The electrode posts are slightly oversized relative to the holes 114 and include a chamfer at their upper ends to facilitate insertion into the holes 114 in the plug where they are maintained in pressed contact with the contact leads for the vaporiser by virtue of the plug.

The end cap has a base section 124 and an upstanding wall 120 which conforms to the inner surface of the housing part 32. The upstanding wall 120 of the end cap 48 is inserted into the housing part 32 so the latch projections 70 engage with the latch recesses 68 in the housing part 32 to snap-fit the end cap 48 to the housing part when the cartridge is assembled. The top of the upstanding wall 120 of the end cap 48 abuts a peripheral part of the plug 44 and the lower face of the spacers 116 on the plug also about the base section 124 of the plug so that when the end cap 48 is attached to the housing part it presses against the resilient part 44 to maintain it in slight compression.

The base portion 124 of the end cap 48 includes a peripheral lip 126 beyond the base of the upstanding wall 112 with a thickness which corresponds with the thickness of the outer wall of the housing part at the interface end of the cartridge. The end cap also includes an upstanding locating pin 122 which aligns with a corresponding locating hole 128 in the plug to help establish their relative location during assembly.

The dividing wall element 36 comprises a single moulding of polypropylene and includes a dividing wall 130 and a collar 132 formed by projections from the dividing wall 130 in the direction towards the interface end of the cartridge. The dividing wall element 36 has a central opening 134 through which the outlet tube 38 passes (i.e. the dividing wall is arranged around the outlet tube 38). In some embodiments, the dividing wall element 36 may be integrally formed with the outlet tube 38. When the cartridge is assembled, the upper surface of the outer wall 102 of the plug 44 engages with the lower surface of the dividing wall 130, and the upper surface of the dividing wall 130 in turn engages with the projections 66 on the inner surface of the outer wall 64 of the housing part 32. Thus, the dividing wall 130 prevents the plug from being pushed too far into the housing part 32—i.e. the dividing wall 130 is fixedly located along the longitudinal axis of the cartridge by the protrusions 66 in the housing part and so provides the plug with a fixed surface to push against. The collar 132 formed by projections from the dividing wall includes a first pair of opposing projections/tongues 134 which engage with corresponding recesses on an inner surface of the outer wall 102 of the plug 44. The protrusions from the dividing wall 130 further provide a pair of cradle sections 136 configured to engage with corresponding ones of the cradle sections 112 in the part 44 when the cartridge is assembled to further define the opening through which the aerosolisable material transport element passes.

When the cartridge 2 is assembled an air channel extending from the air inlet 50 to the aerosol outlet 60 through the cartridge is formed. Starting from the air inlet 50 in the side wall of the housing part 32, a first section of the air channel is provided by the gap 76 formed by the double-walled section 74 in the outer wall 64 of the housing part 32 and extends from the air inlet 50 towards the interface end 54 of the cartridge and past the plug 44. A second portion of the air channel is provided by the gap between the base of the plug 44 and the end cap 48. A third portion of the air channel is provided by the hole 106 through the plug 44. A fourth portion of the air channel is provided by the region within the inner wall 104 of the plug and the outlet tube around the vaporiser 40. This fourth portion of the air channel may also be referred to as an aerosol/aerosol generation region, it being the primary region in which aerosol is generated during use. The air channel from the air inlet 50 to the aerosol generation region may be referred to as an air inlet section of the air channel. A fifth portion of the air channel is provided by the remainder of the outlet tube 38. A sixth portion of the air channel is provided by the outer housing inner tube 62 which connects the air channel to the aerosol outlet 60, which is located at an end of the mouthpiece 33. The air channel from the aerosol generation region to be the aerosol outlet may be referred to as an aerosol outlet section of the air channel.

Also, when the cartridge is assembled a reservoir 31 for aerosolisable material is formed by the space outside the air channel and inside the housing part 32. This may be filled during manufacture, for example through a filling hole which is then sealed, or by other means. The specific nature of the aerosolisable material, for example in terms of its composition, is not of primary significance to the principles described herein, and in general any conventional aerosolisable material of the type normally used in electronic cigarettes may be used. The present disclosure may refer to a liquid as the aerosolisable material, which as mentioned above may be a conventional e-liquid. However, the principles of the present disclosure apply to any aerosolisable material which has the ability to flow, and may include a liquid, a gel, a gas or a solid phase material, where for a solid phase material a plurality of solid particles may be considered to have the ability to flow when considered as a bulk.

The reservoir is closed at the interface end of the cartridge by the plug 44. The reservoir includes a first region above the dividing wall 130 and a second region below the dividing wall 130 within the space formed between the air channel and the outer wall of the plug. The aerosolisable material transport element (capillary wick) 42 passes through openings in the wall of the air channel provided by the semi-circular recesses 108, 90 in the plug 44 and the outlet tube 38 and the cradle sections 112, 136 in the plug 44 and the dividing wall element 36 that engage with one another as discussed above. Thus, the ends of the aerosolisable material transport element extend into the second region of the reservoir from which they draw aerosolisable material through the openings in the air channel to the vaporiser 40 for subsequent vaporisation.

In normal use, the cartridge 2 is coupled to the aerosol provision device 4 and the aerosol provision device activated to supply power to the cartridge via the contact electrodes 46 in the end cap 48. Power then passes through the connection leads 41 to the vaporiser 40. The vaporiser is thus electrically heated and so vaporises a portion of the aerosolisable material from the aerosolisable material transport element in the vicinity of the vaporiser. This generates aerosol in the aerosol generation region of the air path. Aerosolisable material that is vaporised from the aerosolisable material transport element is replaced by more aerosolisable material drawn from the reservoir by capillary action. While the vaporiser is activated, a user inhales on the mouthpiece end 52 of the cartridge. This causes air to be drawn through whichever aerosol provision device air inlet 14 aligns with the air inlet 50 of the cartridge (which will depend on the orientation in which the cartridge was inserted into the aerosol provision device receptacle 8). Air then enters the cartridge through the air inlet 50, passes along the gap 76 in the double-walled section 74 of the housing part 32, passes between the plug 44 and the end cap 48 before entering the aerosol generation region surrounding the vaporiser 40 through the hole 106 in the base part 100 of the plug 44. The incoming air mixes with aerosol generated from the vaporiser to form a condensation aerosol, which is then drawn along the outlet tube 38 and the housing part inner 62 before exiting through the mouthpiece outlet/aerosol outlet 60 for user inhalation.

From the above FIGS. 1-6B, it can be seen a possible embodiment construction of aerosol provision system 1 which is configured for generating an aerosol, which is suitable for use in the context of the present disclosure (alongside potentially other forms of aerosol provision system).

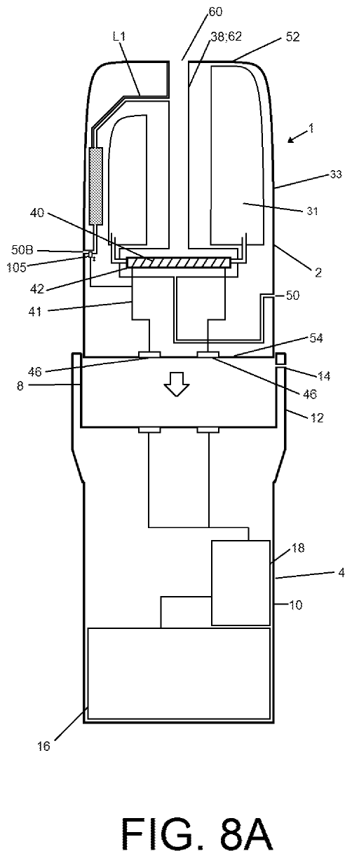

Turning now to FIGS. 7-11, the present disclosure also provides for other applications and embodiments of aerosol provision system 1 which may complement those embodiments of aerosol provision system illustrated and described above with reference to FIGS. 1-6B.

Thus with initial reference to FIGS. 7-9, there is provided in accordance with some embodiments an aerosol provision system 1 for generating an aerosol. The aerosol provision system comprises a first reservoir 31 for storing a first aerosol-generating material, wherein the aerosol provision system 1 is configured to generate a first aerosol using the first aerosol-generating material. This is thus as per some of the earlier embodiments, such as those shown in any of FIGS. 1-6B. In these latter embodiments however, the aerosol provision system 1 also comprises a second reservoir 101 for storing a second aerosol-generating material, wherein the aerosol provision system 1 is configured to generate a second aerosol using the second aerosol-generating material. At a general level therefore, the aerosol provision system with respect to these embodiments may be configured to generate the first aerosol independently of, and in some embodiments simultaneously to, the second aerosol.

By the term independently here, this may be understood as meaning that the first aerosol may be generated in a way which then allows the second aerosol to then be either added to, mixed with, and/or supplied alongside the first aerosol. Put differently, this could thus also mean that the aerosol provision system is configured to generate the second aerosol downstream of the first aerosol, such that any formation of the second aerosol does not impact any initial generation of the first aerosol by the aerosol provision system. Accordingly, the presence of this second reservoir thus allows the user to effectively customise to what extent this first aerosol is supplemented with the second aerosol as part of an end aerosol which is delivered to the user. This being said, the term independently here may also cover instances where the first aerosol is still generated irrespective of how the second aerosol is being generated, but still could cover instances where the second aerosol is nonetheless supplied to the first reservoir. In this way, the first aerosol may still generate the first aerosol independently of the second aerosol; it would just be the case in these instances that any generated second aerosol would then be supplied to the first reservoir as part of the first aerosol generation.

Noting the above, and starting with the first aerosol, it may be appreciated in accordance with some embodiments that the aerosol provision system may comprise an aerosol generator 40, such as a heating element or some other type of aerosol generator (e.g. using vibrational, mechanical, pressurisation or electrostatic means), for generating the first aerosol.

In accordance with some embodiments, the aerosol provision system 1 may be configured to generate the first aerosol at a first temperature, and be configured to generate the second aerosol at a second temperature, wherein the first temperature is greater than the second temperature. In this respect, and as noted previously, it has been recognised that some flavouring materials and/or additives which are configured to be delivered to a user as an aerosol are more effectively aerosolised at lower temperatures than other flavouring materials and/or additives forming part of the delivered aerosol to the user. In this way therefore, to reduce any impairment of these more delicate flavouring materials and/or additives, these may be aerosolised as part of the second, lower temperature, aerosol rather than being aerosolised as part of the first aerosol.

It will be appreciated in accordance with some of these embodiments that the temperature at which either of the first and/or the second aerosol is configured to be generated may vary depending on the intended composition of each of the first and second aerosol generating materials. In accordance with some embodiments however, the aerosol provision system 1 may be configured to generate the second aerosol at a temperature of no more than 50 degree Celsius, and preferably no more than 40 degree Celsius for allowing this second aerosol to better accommodate more delicate flavouring materials and/or additives which are configured to be delivered to a user as an aerosol.

Tying in with the above, and in accordance with some embodiments, the aerosol provision system 1 may be configured to generate the second aerosol (simultaneously to the first aerosol, in most cases) without actively heating the second aerosol generating material. This may be achieved, for instance, by employing an aerosol generator to generate the second aerosol which is not a heating element, such as through using vibrational or means.

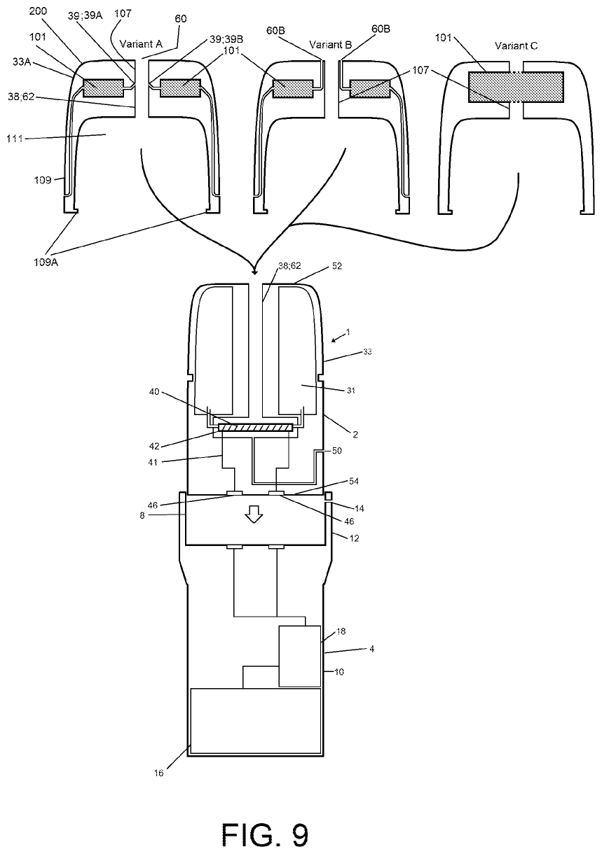

Equally however, the second reservoir may be configured to be at least partially heated, and/or at least passively heated, by the first aerosol for providing a gentle amount of heat which is sufficient enough to generate the second aerosol (as shown in the embodiments of FIG. 7 and FIG. 9 (variant C) for instance. However, in some embodiments, the second reservoir may have its own aerosol generator 40, which may be separate to the aerosol generator 40 relating to the first reservoir, and which may appreciably be powered using power from the power source 16 (which is potentially fed to this aerosol generator(s) 40 via the contact electrodes 46).

To help ensure that the first aerosol is sufficiently hot, and to assist with ensuring there is enough temperature in the first aerosol (for potential use in passively heating the second reservoir where this passive heating is employed), in accordance with some embodiments, the aerosol provision system may be configured to generate the first aerosol at a temperature of at least 15 degrees Celsius, or more limitedly of at least 20 degrees Celsius, or more limitedly of at least 25 degrees Celsius, or more limitedly of at least 30 degrees Celsius, such as by using a heating element as part of the aerosol generator 40.

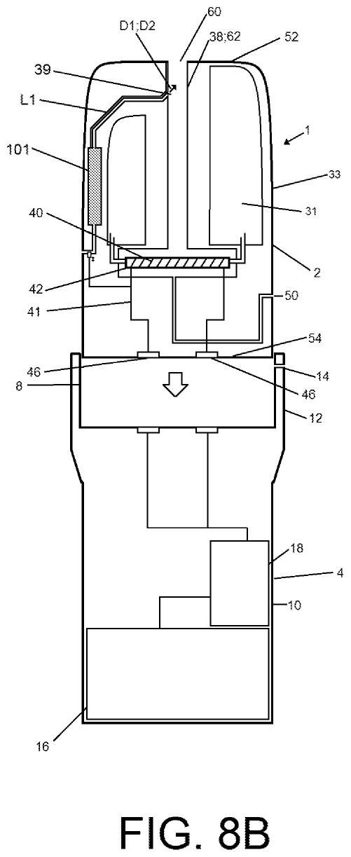

Irrespective of whether any active/passive heating is employed in the aerosol provision system 1 however, e.g. in respect of any of the reservoirs 31;101 therefrom, in at least some embodiments of the aerosol provisions system 1, the second reservoir 101 may be downstream of the first reservoir 31. In this way, the second aerosol may be configured to be imparted to a location which is downstream of where the first aerosol is generated, and potentially in a position where the second aerosol is delivered into the first aerosol. Such embodiments are shown in the embodiments of FIGS. 7, 8A-8B, and some of the embodiments from FIG. 9, where the second aerosol is effectively imparted to the first aerosol in a location which is downstream of where the first aerosol is generated.

Tying in with at least some of the embodiments, for more effectively transferring some residual heat to the second reservoir, and also to allow for better mixing of the second aerosol into the first aerosol, the aerosol provision system in accordance with some embodiments (as shown in the embodiment of FIG. 7, for instance) may be configured to supply the first aerosol through the second reservoir 101.

Whatever the construction/location of any provided second reservoir 101 however, the aerosol provision system 1 may be provided with an aerosol outlet channel 38;62 for receiving the first aerosol form the aerosol generator 40. Although not necessarily, to help contain the first aerosol though this channel, and to help provide a more uniform flow there-through, the aerosol outlet channel 38;62 in accordance with some embodiments may comprise an aerosol outlet tube.

With the provision of the aerosol outlet channel 38;62, for better guiding the second aerosol to the end user of the aerosol provision system 1, in some embodiments (as shown in the embodiments of FIGS. 8A and 8B, for instance) the aerosol provision system 1 may be configured to supply the second aerosol into the aerosol outlet channel 38;62. Doing so may also allow for an improved mixing of the second aerosol with the first aerosol, such for allowing a more uninform mixed aerosol which is delivered to the user.

In some instances, the aerosol provision system 1 may be configured to supply the second aerosol into the aerosol outlet channel 38;62 via at least one opening 39 in the aerosol outlet channel 38;62. For allowing a more improved flow of the second aerosol into the aerosol outlet channel 38;62 in some instances, the at least one opening may in accordance with some embodiments comprises a plurality of openings 39A;39B, as shown in the embodiment of FIG. 9 (variant A) for instance. In accordance with some very particular embodiments, the plurality of openings may be located about different circumferential positions around the aerosol outlet channel 38;62. In this respect as well, and for better ensuring the second aerosol is imparted into the aerosol outlet channel 38;62 more evenly, the plurality of openings in accordance with some embodiments may be all located at a same distance along a length of the aerosol outlet channel 38;62. Phrasing this in other ways, in accordance with some embodiments, the aerosol provision system 1 in some of these embodiments may be configured to supply the second aerosol around the aerosol outlet channel.

Staying with the plurality of openings, where these are employed, in accordance with some embodiments, the plurality of openings may comprise at least four openings, at least six openings, at least eight openings, and/or at least ten openings. By increasing the number of openings, this may thus allow for a more uniformed flow of second aerosol into the aerosol outlet channel 38;62, and also may provide improved reliability of the aerosol provision system in the event that one of the plurality of openings becomes inadvertently blocked.

Where any opening(s) 39 is employed, it will be appreciated that the size of each opening may be configured depending on the intended exact application of the opening for a given first and/or second aerosol. Though at a general level however, it has been found that each opening comprising a cross-sectional area of no more than 25 mm², or more limitedly no more than 15 mm², or even more limitedly no more than 10 mm², has been found to be particularly suitable in the context of aerosol provision systems 1 of some of the types/sizes herein described which may be used, for instance, as part of a system comprising a consumable 2 and an aerosol provision device 4 which is configured to receive the consumable 2.

Whatever the internal construction of the aerosol provision system 1 relating to any employed aerosol outlet channel 38;62, the aerosol outlet channel 38 at a general level may be configured to supply the first aerosol to a (first) aerosol outlet 60 from the aerosol provision system 1—about which the user can then inhale the first aerosol. In this way therefore, in some embodiments, any provided opening(s) 39 may in some instances be located upstream of the (first) aerosol outlet 60, such to allow the first aerosol and the second aerosol to be inhaled by the user about this aerosol outlet 60;60A. In such embodiments, for better directing the flow of the second aerosol towards this outlet, and to improve the uniformity of the aerosol in the proximity of the aerosol outlet 60;60A, in some of these embodiments (as shown in the embodiment of FIG. 8B for instance), the aerosol provision system 1 may be configured to supply the second aerosol into the aerosol outlet channel 38;62 in a direction D1 which extends towards the aerosol outlet 60, and/or a direction D2 which extends away from the aerosol generator 40.

Appreciably however, and in accordance with some embodiments (as shown in variant B from FIG. 9), the aerosol provision system may be configured to keep the second aerosol separate from the first aerosol for a longer period of time, such that in accordance with some embodiments, the aerosol provision system 1 may be configured to supply the second aerosol to a second aerosol outlet 60B (which may be separate from the first aerosol outlet 60A). Related to this functionality, and potentially other embodiments too, the aerosol provision system 1 in some of these embodiments may be configured to supply the second aerosol around the aerosol outlet channel 38;62, e.g. as part a concentric arrangement in some embodiments such that in some very narrow embodiments, the second aerosol may be configured to flow around (or at least partially around) the outside of, or flow concentrically around, the aerosol outlet channel 38;62. Such a particular embodiment is shown in variant B from FIG. 9, for example. By virtue of this arrangement, the second aerosol may be kept isolated from the first aerosol for a longer period of time, which may be preferable in embodiments where the aerosol provision system is configured to supply the second aerosol (potentially through the second aerosol outlet 60B) at a different (faster or slower) rate than the rate at which the first aerosol is configured to be supplied (potentially through the aerosol outlet 60;60A). Thus related to these embodiments, it may be seen that in some instances where the second aerosol outlet 60B is employed, any such second aerosol outlet may in some instances be annular.

Appreciably however, in so far as any second aerosol outlet is employed, in some instances where the second aerosol is configured to with the first aerosol, e.g. through the at least one opening 39 in the aerosol outlet channel 38;62, in such embodiments the first aerosol outlet 60;60A may be effectively downstream of the second aerosol outlet 60B, and/or such that the second aerosol outlet 60B comprises the at least one opening 39 in the aerosol outlet channel 38;62.

With respect to each of the generated first and second aerosol, it may be seen that there may be provided an air inlet for supplying air, to each of the first reservoir 31 and the second reservoir 101, for generating the respective first aerosol and the second aerosol. Thus at a general level, the aerosol provision system 1 may be configured to generate the first aerosol using air supplied from a first air inlet 50 from the aerosol provision system 1 (e.g. the air inlet 50 as described previously with respect to some of the aerosol provision systems shown in FIGS. 1-6B). The aerosol provision system 1 may be then configured to generate the second aerosol using air supplied from a second air inlet 50B from the aerosol provision system 1, and/or be configured to generate the second aerosol using air supplied from the first inlet 50 (as shown in the embodiment of FIG. 7, where the air from the (first) air inlet 50 passes through the second reservoir 101). Where any second air inlet 50B is employed, appreciably in some embodiments that first air inlet 50 may be separate from the second air inlet 50B. By keep the two air inlets separate, this may assist with providing a fresh source of air to the second reservoir, and may also allow for different flow rates of air to be delivered to each of the first reservoir 31 for generating the first aerosol, and the second reservoir 101 for generating the second aerosol.

To help allow the aerosol provision system 1 to be used with a user's lip, for providing a good seal around any provided aerosol outlet(s) 60;60B, which may in some embodiments also correspond to a mouthpiece outlet 60;60B, the aerosol provision system may comprise a mouthpiece 33 (again, like in some of the embodiments from FIGS. 1-6B).

For assisting with providing a shorter flow path of air/second aerosol, in so far as a second air inlet 50B may be employed, the second air inlet in some embodiments may be located more proximal to the mouthpiece 33 (and/or a first mouthpiece outlet 60 or second mouthpiece outlet 60B) than the first air inlet 50 is located to the mouthpiece 33 (and/or the first mouthpiece outlet 60 or the second mouthpiece outlet 60B).

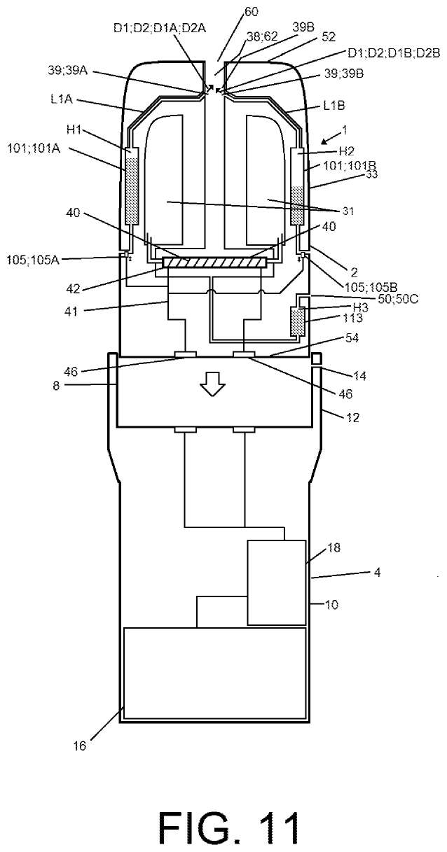

In this respect as well, noting the second aerosol may be configured for use with more delicate flavouring materials and/or additives, in some embodiments the minimum length L1 of the flow path between the second reservoir and the second (mouthpiece) aerosol outlet 60B, or to the first aerosol outlet 60 where the first aerosol is configured to mix with the second aerosol may be no more than 50 mm, preferably no more than 40 mm, more preferably no more than 30 mm, or even more preferably no more than 25 mm. By the phrase ‘minimum length’ here, this may be understood as meaning the shortest distance through which an aerosol may need to travel from the second reservoir 101 to reach the relevant outlet of the aerosol provision system, e.g. as noted by the pertinent distances L1;L1A;L1B as noted in the Figures (noting where more than two second reservoirs 101 are employed, one of these (e.g. primary) second reservoir may have a different minimum length than another (e.g. second) of these second reservoirs, e.g. as shown in the embodiment of FIG. 11, to cater for the particular different aerosol generating material properties relating to each of these different second reservoirs). Thus by keeping this distance to be relatively short, this may assist with preventing the second aerosol from condensing/decomposing before it reaches the relevant outlet (where the user's mouth is) from the aerosol provision system, such to ensure the second aerosol is preserved as best possible for the end user.

With respect to the composition of each of the first aerosol and the second aerosol, it is noted that these may be tailored to the specific application of the aerosol provision system 1, as required. For instance, the first aerosol may comprise any aerosol as described previously with respect to the embodiments from FIGS. 1-6B, for instance, such that the first aerosol, and/or the first aerosol generating material, may in some instances comprise a flavouring material, such as menthol; tobacco; and/or nicotine.

The same may be said for the second aerosol, in so far as the second aerosol may notionally comprise any aerosol as described previously with respect to the embodiments from FIGS. 1-6B. For instance, the second aerosol, and/or the second aerosol generating material, may in some instances comprise a flavouring material, such as menthol; tobacco; and/or nicotine. In this respect, a potential first application for the second aerosol generating material is to act as a supplement (or potentially an optional, or controllable, supplement as will be described) to modify or boost the first aerosol with further/different flavouring materials or additives using the second aerosol. For instance, in a very particular embodiment, the second aerosol generating material and/or the second aerosol may be configured to comprise a flavouring material comprising menthol or mint, or some other flavouring material including nicotine. In this way, the second aerosol may thus allow the user to deliver these additional flavouring materials or additives as part of the end aerosol which is delivered to the user. This may be particularly beneficial, from a retrofitting perspective as will be described, where the first aerosol and/or the first reservoir, may not be configured to deliver these flavouring materials delivered as part of the second aerosol. Thus the provision of the second aerosol in these instances has been found to greatly increase the versatility of the aerosol provision system. A further benefit of this second aerosol is its potential use to allow the user to modify a property of the aerosol which is delivered to the user. Indeed, at a most basic level, the second aerosol may not contain any flavouring material, but could instead just act as a tool for better controlling the flow or thermal properties of the aerosol which is delivered to the user (for instance, by using a cooler/faster aerosol, to cool/modify the properties of the aerosol otherwise delivered to the user form an otherwise hotter/slower first aerosol.

With respect to some of the above embodiments, the aerosol provision system may be configured to selectively prevent generation of the second aerosol from the second aerosol generating material, or may be configured to vary the rate at which second aerosol is generated from the second aerosol generating material. In this way, and as noted above, the second aerosol may thus be used as refining measure to selectively control the properties (or flavour) of the end aerosol which is delivered to the user. Two particular examples of this could include: i) the second aerosol comprising a flavouring material comprising menthol or a mint flavour (or some other flavouring material), and the aerosol provision system being configured to selectively prevent generation of the second aerosol from the second aerosol generating material. In this way, the user may be then able to control when the second aerosol is generated, e.g. in instances where the user requires a menthol/mint flavouring, and/or the user requires a temporary boost of nicotine; and/or ii) the second aerosol comprising one or more of a flavouring material, nicotine, cannabinoids, or some other medicament. In this way, the user may be configured to vary the rate at which the second aerosol is generated from the second aerosol generating material, for allowing the use to selectively control how much of this flavouring material, or medicament, is delivered to them (e.g. to control how intense the flavour of the aerosol should be).

It is to be noted, as set out above, that the second aerosol, and/or the second aerosol generating material, could in some instances comprise a medicament. This may thus be suitable for allowing the aerosol provision system to be retrofitted as a medicament dispenser, which can dispense the medicament as part of the second aerosol in a controlled manner, compared with trying to dispense the medicament as part of the first aerosol which may in some embodiments not be possible if the system is intended to be retrofitted to an existing aerosol provision system 1 comprising the first reservoir 31 which is otherwise not configured for use with such a particular medicament.

Thus in some embodiments, the aerosol provision system 1 may be configured to selectively prevent generation of the second aerosol from the second aerosol generating material, or may be configured to vary the rate at which second aerosol is generated from the second aerosol generating material. As to how this is achieved, this may appreciably be achieved through mechanical means and/or electrical means as required. For instance, a particular embodiment for varying the rate at which second aerosol is generated from the second aerosol generating material may be through the aerosol provision system comprising a throttling portion 105 for varying at least one of the rate of generation of the second aerosol, and/or the rate at which the second aerosol is configured to exit the aerosol provision system. With respect to this throttling portion 105 therefore, depending on its location, the throttling portion may be configured to control the flow of air to the second reservoir 101, and/or control the flow of second aerosol form the second reservoir 101 towards the user/second (mouthpiece) outlet 60B and/or the first mouthpiece outlet 60A. Where such a throttling portion is provided, the throttling portion could appreciably comprise a mechanical throttling portion, such as a moveable member inside the air/aerosol flow path relating to the second reservoir 101, such as being located just downstream of the second air inlet 50B as shown in FIG. 8A, and/or could be located upstream of the at least one opening 39 where such an opening 39 is provided, and/or between the at least one opening 39 and the second reservoir 101 (to avoid the throttling portion 105 from affecting the flow of the first aerosol from the first reservoir 31).

With respect to any employed throttling portion 105, this may also be achieved through the second air inlet 50B comprising an adjustable size, and the throttling portion 105 comprising the second air inlet 50B, such that the throttling portion is effectively configured to control a flow rate of air which is delivered to the second reservoir 101. Other embodiments for the throttling portion could also include the throttling portion 105 comprising a variable sized orifice/second air inlet 50B from the aerosol provision system, which may in some instances be user adjustable (e.g. in the form of a simple latch/slider, which can be adjusted by the user to control the size of the orifice or second air inlet 50B).

Appreciably however, the aerosol provision system 1 may be configured to selectively prevent generation of the second aerosol from the second aerosol generating material, or may be configured to vary the rate at which second aerosol is generated from the second aerosol generating material, without the use of a throttling portion 105 necessarily. For instance, and where a second aerosol generator 40 is provided for generating the second aerosol, the aerosol provision system 1 may be configured to vary the power delivered to the second aerosol generator 40, as required, to control the provision/generation of the second aerosol.

At a general level, a pressure reduction will conventionally be observed within the aerosol provision system 1 as the user draws on the system, causing air to pass through one or more air inlets, via one or more reservoirs, before exiting the system via one or more outlet channels, i.e. air/aerosol flow paths within the system. Such pressure drops may allow sufficient aerosol/vapour, including desirable active substances such as flavour compounds, to be inhaled by the consumer. It may also be appropriate to configure or design the air/aerosol flow paths present within the aerosol provision system 1 with certain characteristics, particularly when considering the form/nature of the aerosol-generating material(s) and/or active substances(s) contained within the reservoirs. For example, relating to any throttling techniques such as those outlined above, valves/orifices/throttling portions may be employed to alter the ratio of aerosol-generating material(s) and/or active substances(s) delivered to the user based upon the contents of each respective reservoir, e.g. differing nicotine concentrations, flavour materials and/or sensates. Such an embodiment may be adjusted, for example, by the control circuitry 18 for controlling and monitoring the operation of the electronic cigarette based upon environmental conditions, or user operation (e.g. number or length of draws on the aerosol provision system 1). A further example of such an embodiment may be the active control by the user during use, for example by using a user input button, such as the user input button 20, to increase/decrease/change aerosol-generating material(s) and/or active substances(s) concentrations delivered by the system to the user.

Noting the above, and in accordance with some embodiments, the total pressure difference across the reservoirs is from greater than 0 to 200 or less mm water gauge (mmWG). In some embodiments, the total pressure difference across the reservoirs is from about 50 to about 175 mmWG. In some embodiments, the total pressure difference across the reservoirs is from about 60 to about 160 mmWG. In some embodiments, the total pressure difference across the reservoirs is from about 70 to about 150 mmWG. In some embodiments, the total pressure difference across the reservoirs is from about 80 to about 140 mmWG. In some embodiments, the total pressure difference across the first reservoir and the second reservoir is from greater than 0 to 200 or less mmWG. In some embodiments, the total pressure difference across the first reservoir and the second reservoir is from about 50 to about 175 mmWG. In some embodiments, the total pressure difference across the first reservoir and the second reservoir is from about 60 to about 160 mmWG. In some embodiments, the total pressure difference across the first reservoir and the second reservoir is from about 70 to about 150 mmWG. In some embodiments, the total pressure difference across the first reservoir and the second reservoir is from about 80 to about 140 mmWG.

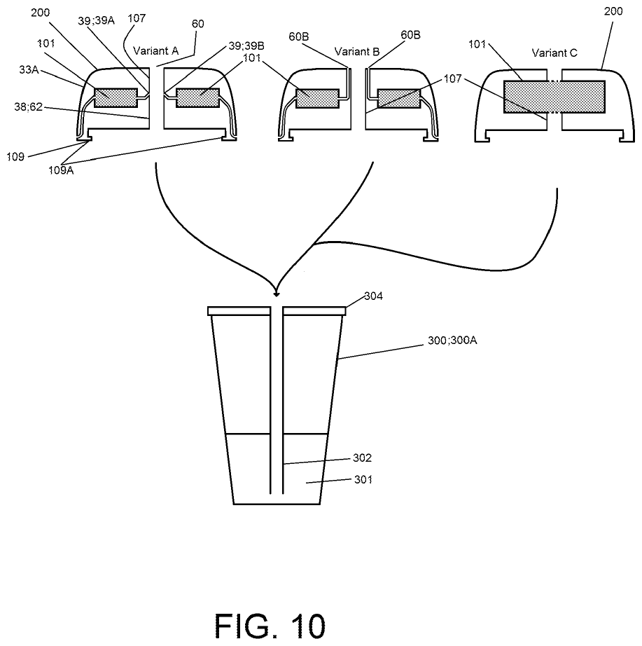

Moving away from the possible formation/uses of the second aerosol, and any related pressure drops relating to any related reservoirs, such as the first and second reservoirs, and turning to the structure of any potential employed aerosol provision system 1 using such a second aerosol, a particularly space saving arrangement may be through the second reservoir 101 at least partially surrounding the aerosol outlet channel 38;62, and/or such that the second reservoir 101 is annular (as shown in the embodiments from FIG. 7, or variants A and B from FIG. 9, for instance), or such that the second reservoir 101 is configured to removably fit/locate in a recess of the aerosol provision system 1. This may be further advantageous in more conveniently allowing the second reservoir 101 to be retrofitted to an existing aerosol provision system as part of a module 200, as will be described—but as is shown in the embodiments of FIGS. 9 and 10 in the meantime). In some space saving embodiments, one of the first reservoir and the second reservoir may at least partially surround the other of the first reservoir and the second reservoir, again as shown in the embodiments from the Figures.

As to the size of each provided first reservoir and the second reservoir, these may appreciably be varied depending on the intended application of the aerosol provision system 1. However, and in so far as the aerosol provision system may intended to be relatively portable, and placed in a pocket when not in use, the first reservoir 31 and/or the second reservoir 101 may comprise a volumetric capacity of no more than 50 ml; no more than 40 ml; no more than 30 ml; no more than 25 ml; no more than 20 ml; or at the most space saving/portable end no more than 15 ml. Notionally, in embodiments where the second reservoir is for use as more of a top-up, or booster, to supplement the content of the first aerosol with other flavourings; additives; or even a medicament, such that the consumption of second aerosol generating material in the second reservoir may typically be less than the consumption of first aerosol generating material in the first reservoir over time, in some embodiments the volumetric capacity of the second reservoir may be less than the volumetric capacity of the first reservoir (e.g. such that the first reservoir may comprise a volumetric capacity of no more than 40 ml, or no more than 50 ml, and the second reservoir may comprise a volumetric capacity of no more than 25 ml, or no more than 20 ml, for instance).

Although not necessarily, it may be seen that the herein described aerosol provision systems 1 may be provided as part of a system which comprises the previously described consumable 2 and aerosol provision device 4 (as shown in the embodiments of FIGS. 1-6B, for instance)—where the consumable 2 is configured to releasably couple to the aerosol provision device, e.g. via the consumable receiving section/receptacle 8 from the aerosol provision device 4. In such embodiments therefore, it may be seen that the consumable (as shown in some of the embodiments from FIGS. 7-8B at least) may comprise at least a portion of the aerosol outlet channel 38;62; the first reservoir 31; the second reservoir 101; the mouthpiece 33; and/or an aerosol/mouthpiece outlet 60 for outputting at least the first aerosol (and, in some instances, for outputting a mixture of the first aerosol and the second aerosol where these two aerosol are configured to mix inside the consumable before reaching the aerosol/mouthpiece outlet 60.

Noting the above however, potential applications of the herein described second reservoir 101, as alluded to previously, include the use of this second reservoir 101 being used as part of a retrofitting type arrangement, whereby the second reservoir 101 can be used a bolt-on to an existing aerosol provision system such as those shown in FIGS. 1-6B. Thus in accordance with these embodiments/applications, there may be provided a module 200 which comprises the second reservoir 101, and which is configured to receive the first aerosol from an existing aerosol provision system 1. Such a module 200 will now be described with reference to some of the embodiments, and example variants A-C, shown in FIG. 9.

At a general level, the module 200 is configured to be releasably coupled to the aerosol provision system, such as to a portion thereof (e.g. the consumable 2 [where employed], or a mouthpiece 31, of the aerosol provision system 1) for allowing the module to receive the first aerosol from the upstream portions of the aerosol provision system 1. In this way for instance, the module 200 may be configured to receive the first aerosol from the aerosol outlet channel 38;62, and pass this first aerosol into an aerosol outlet channel 107 (which may be thus effectively a continuation of the aerosol outlet channel 38;62), such to allow the first aerosol to pass through the module 200 towards an aerosol/mouthpiece outlet from the module 200.

The module 200 may be configured to be releasably coupled to the aerosol provision system 1 through the provision of an attachment portion 109 from the module 200. As to what this attachment portion 109 might be, it will be appreciated that this may be configured appropriately, depending on the intended application of the module 200. For instance, in the particular embodiment from the variants shown in FIG. 9, the attachment portion 109 may be resilient, and comprise a projection which is configured to engage with a corresponding recess from the aerosol provision system 1 for releasably coupling the module 200 to the aerosol provision system 1. Appreciably however, the attachment portion 109 may equally comprise a recess which is configured to engage with a corresponding projection or part from the aerosol provision system 1 for releasably coupling the module 200 to the aerosol provision system 1, or could even comprise a magnetic portion for engaging with a magnetic portion from the aerosol provision system 1. A yet another potential embodiment for the attachment portion 109 could be where the attachment portion 109 alternatively/additionally comprises a threaded arrangement which is configured to engage with a corresponding threaded arrangement from the aerosol provision system for releasably coupling the module 200 to the aerosol provision system 1, such as to the mouthpiece 33 or consumable 2 therefrom where these are employed.

For assisting with providing a snug, and sealed, coupling between the module and the portion of the aerosol provision system to which the module is attached (e.g. the mouthpiece 33), in some embodiments, the module may define a recess 111 for accommodating a portion of the aerosol provision system, such as the mouthpiece portion of the aerosol provision system as shown in the embodiments of FIG. 9. In some embodiments, the recess may be shaped to match a surface profile of the mouthpiece 33 of the aerosol provision system 1, again as shown in the embodiments of FIG. 9. In this way, the module may be configured to act as a cap which is configured to couple/locate over, or couple/locate to, the mouthpiece 33 from the aerosol provision system 1.

As alluded to above, any employed module 200 may be configured to comprise the second reservoir 101, and any second aerosol generating material therein, such as a flavouring material or a medicament as described previously. Noting this, and the potential application of the module, it is envisaged in some embodiments that the module may be disposable; and/or configured to be consumable, or even configured such that it is intended to be single-use (i.e. the second reservoir is sufficiently small such that it is intended to only provide a single small amount of aerosol before being depleted of second aerosol generating material). In some embodiments however, the second reservoir may be configured to be refillable for allowing the module 200 (or the aerosol provision system 1 more generally, where the module is not employed) to be reused/refilled.

In so far as the module 200 may be configured to be located over a mouthpiece of the aerosol provision system, for coupling purposes, in some embodiments, the module may then comprise its own/second mouthpiece 33A for providing the user with an appropriate surface to place their lips on the module when the module is coupled over the other/existing mouthpiece 33 of the aerosol provision system 1.

Appreciating the provision of such potential modules 200 therefore, it may be seen that provided herein may also thus effectively be corresponding methods of using this module, as well as various retrofitting methods for retrofitting such a method onto an existing aerosol provision system 1. For instance, one such method may include a method of retrofitting an aerosol provision system 1 configured to generate the first aerosol using a first aerosol-generating material received from the first reservoir 31 of the aerosol provision system 1, wherein the method comprises: releasably coupling the module 200, comprising the second reservoir 101 storing the second aerosol-generating material, to the aerosol provision system 1; generating the first aerosol using the first aerosol-generating material; and generating the second aerosol, inside the module 200, using the second aerosol-generating material.

In such methods therefore, the first aerosol could then be passed through the module; and then supplied along with the second aerosol to at least one outlet (including potentially separate outlets for each aerosol per the variant B from FIG. 9) from the module 200. In some variants of the methods as well, the methods could comprise mixing the first aerosol and the second aerosol inside the module (as shown for instance in variants A and C from FIG. 9), or have the first aerosol supplied through the second reservoir (as shown for instance in variant C from FIG. 9). Appreciably therefore, it will be appreciated that the herein described apparatuses and systems may equally be used as part of corresponding methods employing these same apparatuses and systems, for instance as per any of the described method operations as listed in the claims/clauses at the end of this specification.

Appreciating the foregoing therefore, at least, there has been described an aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing a second aerosol-generating material, wherein the aerosol provision system is configured to generate a second aerosol using the second aerosol-generating material;
wherein the aerosol provision system is configured to generate the first aerosol independently of the second aerosol.

There has also been described an aerosol provision system as described above, wherein the aerosol provision system further comprises a consumable and an aerosol provision device, wherein the aerosol provision device comprises a consumable receiving section that includes an interface arranged to cooperatively engage with an interface from the consumable so as to releasably couple the consumable to the aerosol provision device.

There has also been described a consumable for use in an aerosol provision system for generating an aerosol, wherein the consumable comprises:
a first reservoir for storing a first aerosol-generating material, wherein the aerosol provision system is configured to generate a first aerosol using the first aerosol-generating material;
a second reservoir for storing a second aerosol-generating material, wherein the aerosol provision system is configured to generate a second aerosol using the second aerosol-generating material;
wherein the aerosol provision system is configured to generate the first aerosol independently of the second aerosol.

There has also been described a consumable for use in an aerosol provision system for generating an aerosol, wherein the consumable comprises:
a first reservoir for storing a first aerosol-generating material, wherein the aerosol provision system is configured to generate a first aerosol using the first aerosol-generating material;
a second reservoir for storing a second aerosol-generating material, wherein the consumable is configured to generate a second aerosol using the second aerosol-generating material;
wherein the consumable is configured to generate the first aerosol independently of the second aerosol.

There has also been described a method of generating an aerosol in an aerosol provision system, wherein the method comprises:
generating a first aerosol using a first aerosol-generating material received from a first reservoir of the aerosol provision system;
generating a second aerosol using a second aerosol-generating material received from a second reservoir of the aerosol provision system;
wherein the first aerosol is generated independently of the second aerosol.

There has also been described a method of retrofitting an aerosol provision system configured to generate a first aerosol using a first aerosol-generating material received from a first reservoir of the aerosol provision system, wherein the method comprises:
releasably coupling a module, comprising a second reservoir storing a second aerosol-generating material, to the aerosol provision system;
generating a first aerosol using the first aerosol-generating material;
generating a second aerosol, inside the module, using the second aerosol-generating material.

There has also been described a module, for use in an aerosol provision system which is configured to generate a first aerosol using a first aerosol-generating material, wherein the module is configured to be releasably coupled to the aerosol provision system, and wherein the module comprises:
a second reservoir for storing a second aerosol-generating material; and
an attachment portion for releasably coupling the module to the aerosol provision system;
wherein the module is configured for receiving a first aerosol from the aerosol provision system when the module is coupled to the aerosol provision system using the attachment portion, and is configured to generate a second aerosol using the second aerosol-generating material when the module is receiving the first aerosol.

There has also been described an assembly comprising the as described above, and the aerosol provision system to which the module is configured to be releasably coupled.

There has also been described all of the envisaged wide ranging embodiments as noted in the various clauses and claims as recited at the end of this specification.

There has also been described an aerosol provision system 1 for generating an aerosol. The aerosol provision system comprises a first reservoir 31 for storing a first aerosol-generating material, wherein the aerosol provision system 1 is configured to generate a first aerosol using the first aerosol-generating material. The aerosol provision system 1 also comprises a second reservoir 101 for storing a second aerosol-generating material, wherein the aerosol provision system 1 is configured to generate a second aerosol using the second aerosol-generating material. In this way, the first aerosol may be generated in a way which then allows the second aerosol to then be either added to, mixed with, and/or supplied alongside the first aerosol in a way which allows the user to effectively customise to what extent this first aerosol is supplemented with the second aerosol as part of an end aerosol which is delivered to the user.

In order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilised and modifications may be made without departing from the scope of the claims. Various embodiments may suitably comprise, consist of, or consist essentially of, various combinations of the disclosed elements, components, features, parts, steps, means, etc. other than those specifically described herein, and it will thus be fully appreciated that features of the dependent claims (or any herein recited dependent clauses) may be combined with features of the independent claims (or independent clauses) in combinations other than those explicitly set out in the claims or clauses. The disclosure may include other inventions not presently claimed, but which may be claimed in future. Accordingly, any permutation of the features from the claims may be combined as required, and/or may be combined with any permutation of the features from the herein recited clauses at the end of this specification.

For instance, although a number of the herein described aerosol provision systems have been described as employing a consumable 2 and an aerosol provision device 4 type-arrangement, where one part is configured to be releasably coupled to the other part, it will be appreciated that some variants of the aerosol provision system 1 may not employ such a two-part structure, such that all of the components of the consumable 2 and the aerosol provision device 4 may be located as part of a singular component.

Equally, although a number of the herein described aerosol provision systems have been described as employing both a first reservoir (in respect of the first aerosol generating material for generating the first aerosol), and a second reservoir (in respect of the second aerosol generating material for generating the second aerosol), it will be appreciated that the herein described systems and methods may be employed to other systems and methods whereby an additional third/fourth/fifth reservoir (containing a respective third/fourth/fifth aerosol generating material for generating a respective third/fourth/fifth aerosol) is present. In this way, the system/method may be configured for potentially allowing more/different combinations of aerosol to be provided, such as a combination of the first aerosol with any of these second/third/fourth/fifth/nth aerosols. In this way as well, it is envisaged that the first aerosol may be mixed, or supplied separately to/independently of, or together with any of these other aerosols in any required different way/time.

For instance, in one example, there may be provided a system with three reservoirs, whereby the aerosol provision system is configured to allow the user to select from delivering the first aerosol with a second aerosol (e.g. containing a certain flavouring; additive; sensate; or acid, and/or containing cannabinoidsfor instance) via second aerosol generating material from the second reservoir 101, or delivering the first aerosol with a third/different aerosol (e.g. containing a different flavouring (e.g. menthol); additive; sensate; or acid, and/or containing nicotine for instance) via third/different aerosol generating material from a third/different reservoir which is separate from the second reservoir.

Or in another example, there may be provided a system with three reservoirs, whereby the aerosol provision system is configured to allow the user to mix the first aerosol with the second aerosol, but then also provide an optional/toggleable/customisable third reservoir in which one or more acids are provided, wherein the user can use the aerosol provision system to control how much acid is ultimately provided as part of the end aerosol which is delivered to the user (for instance using a throttling portion 105 as part of the third reservoir 113 and any inlet 50C therefor). An example of this arrangement is shown in the example embodiment of FIG. 11, for instance. In any such embodiment as well, and potentially others, there may also be provided a plurality (e.g. a primary and secondary) of second reservoirs 101A;101B, each with its own [different] aerosol generating material in it (e.g. one second reservoir 101A comprising a flavouring material comprising menthol, and another second reservoir 101B comprising a different flavouring material, such as mango). In this way, the aerosol provision system 1 may be configured to allow the user to switch between the two second reservoirs 101A; 101B for selecting which type of second aerosol is formed. The aerosol provision system 1 might then be further configured to allow the user to customise the aerosol delivered from the third reservoir 113 [to the first reservoir 101] to control how much acid [from the third aerosol generating material, which comprises one or more such acids] is delivered to the end user which is delivered to the user, noting the amount of acid delivered has been found to have an effect on the user perception of flavouring materials supplied as part of this same end aerosol to the user (e.g. at the mouthpiece outlet 60).

It may be seen therefore that the herein described techniques may be employed with any number of reservoirs 31;101;101A; 101B;113, each for respectively generating an aerosol which may be individually mixed with aerosols from any number of, or combination of, other reservoirs from the aerosol provision system 1, and in a variety of different orders/locations, as required, for better providing the user with flexibility as to what should be located in their end aerosol which they are configured to inhale via any ultimate/end outlet 60 from the aerosol provision system 1.

Equally as well, it is also envisaged that the herein described module 200 may have applications for retrofitting to any form of existing aerosol provision system 1, and may even have applications for adding an aerosol to any form of existing first aerosol or other fluid (e.g. for application in providing an aerosol to an aerosol from an inhaler for instance), or for adding the aerosol from the module 200 to a fluid source from a container/bottle-such as adding or mixing the aerosol to a fluid 301 located in a fluid outlet channel 302, or fluid outlet tube 302, from a container 300, such as a drinks container 300A, as shown in FIG. 10 for instance.

In this way, and with reference to such embodiments as shown in FIG. 10 for instance, upon the user of the container 300 drawing up fluid through the fluid outlet channel 302 of the container 300, the module 200 (which may be coupled to the lid 304 of the container using its attachment portion 109) may be configured to provide an aerosol (e.g. an aerosol comprising a flavouring agent (such as menthol, or tobacco)), or even a medicament, to the user at the same time as the fluid being delivered to the user. In this way therefore, the module 200 may serve as an incredibly useful module for supplying flavouring agents and/or additives, or even medicament, as an aerosol to the user of the container 300 alongside any fluid being dispensed therefrom.

For completeness as well, it is be noted that although the herein described aerosol provision systems 1 have been described as potentially heating at least one of the first and/or second reservoirs, it is be noted that either of these reservoirs may be configured to be equally cooled and/or not heated for generating the respective aerosol relating to that reservoir, via use of an appropriate aerosol generator and/or aerosol generating material which is configured to create the appropriate aerosol in these reservoirs without the use of heat (e.g. through using a vibrational, mechanical, pressurisation or electrostatic type aerosol generator 40, or physical means, as described previously).

With respect to generating each of the herein described aerosols as well, whether this be in respect of using the first aerosol generating material from the first reservoir; the second aerosol generating material from the second reservoir; and/or any provided third/fourth/fifth/nth aerosol generating material from the third/fourth/fifth/nth reservoir, it has also been found that adding an appropriately sized headspace H1;H2;H3 to each of these reservoirs may assist with the formation of aerosol therein. By the term ‘headspace’, as shown in the embodiment of FIG. 11 for instance, this may be understood as meaning a space inside the reservoir 31;101;113 which is otherwise vacant/unoccupied (e.g. just a space occupying air) with other parts from the reservoir (such as the aerosol generating material relating to that reservoir; and/or such as relating to any aerosolisable material transport element or substrate from the reservoir which is used to otherwise accommodate the aerosol generating material relating to that reservoir). Thus with the provision of this ‘empty’ headspace in the reservoir, this space may thus allow for improved aerosol generation inside the reservoir, through providing the aerosol with a more sufficient space in which to form the aerosol. As to the extent of this potential headspace, where employed, it is envisaged that in some particularly effective embodiments, any combination (or each) of the herein described reservoir(s) may comprise a headspace H1;H2;H3 which is no smaller than 5% of the total volumetric capacity of the reservoir; or more limitedly no smaller than 10% of the total volumetric capacity of the reservoir; or more limitedly no smaller than 15% of the total volumetric capacity of the reservoir; and/or more limitedly no smaller than 20% of the total volumetric capacity of the reservoir; or more limitedly no smaller than 25% of the total volumetric capacity of the reservoir; or more limitedly no smaller than 30% of the total volumetric capacity of the reservoir; or more limitedly no smaller than 40% of the total volumetric capacity of the reservoir; or more limitedly no smaller than 50% of the total volumetric capacity of the reservoir; or more limitedly no smaller than 60% of the total volumetric capacity of the reservoir; or more limitedly no smaller than 70% of the total volumetric capacity of the reservoir; or more limitedly no smaller than 80% of the total volumetric capacity of the reservoir; or more limitedly no smaller than 90% of the total volumetric capacity of the reservoir. It will be appreciated in some embodiments that the headspace of one reservoir may be different to the headspace of a different reservoir, to better match the intended aerosol being generated for that particular reservoir.

For completeness as well, it will also be appreciated that any employed second reservoir 101 may have particular application where it comprises, at a very general level, an active substance which is configured to be delivered to a user of the aerosol provision system, as part of any first aerosol delivered to the user using the first aerosol-generating material. In this way, the active substance may effectively serve to enhance, augment or otherwise alter a property; characteristic; flavour; and/or effect of the first aerosol, depending on the particular active substance(s) which are employed, and may also help more fundamentally to help provide one or more physiological effects on the user of the aerosol provision system as part of the first aerosol delivery.

By the term active substance here, this is intended to cover any substance (or combination of substances) which may impart a physiological effect on the user of the aerosol provision system. Thus with reference to some of the terminology as used herein, any employed active substance may comprise, for example, any combination of a flavouring material; nicotine; one or more cannabinoids, such as tetrahydrocannabinol and/or cannabidiol; one or more sensates, such as a cooling agent; a warming agent; and/or a tingling agent.

Where the active substance comprises a flavouring material, it will be appreciated that this material enhance the flavour imparted on the user via the first aerosol.

Whatever the active substance(s) which is employed, it will be appreciated that in some embodiments, the active substance may be configured to be delivered to the user during use of the aerosol provision system 1, and/or during generation of the first aerosol. This delivery may be through delivering a portion of the active substance to at least one outlet from the aerosol provision system, such as an outlet on the mouthpiece 33, which is in fluid communication with the second reservoir 101.

Although not necessarily, in some embodiments, the active substance may be configured to be delivered to the outlet as part of a second aerosol or a vapour, such as per some of the methodologies discussed previously with respect to any of FIGS. 7-11 at least.

Where such an at least one outlet is employed, each such outlet in some embodiments may be located on an external surface of the mouthpiece 33, and/or which may be an external surface which the user's mouth is configured to touch or cover when the mouthpiece 33 is in use. This at least one outlet could be in fluid communication with the aerosol outlet 60, and/or could be an outlet(s) which is separate from, yet potentially located proximate to, or located around, or at least partially surrounding the aerosol outlet 60.

By way of the above therefore, and at a general level, the aerosol provision system 1 may be configured to deliver the active substance, potentially (but not necessarily) as an aerosol/vapour, to the at least one outlet of the mouthpiece 33 during use of the aerosol provision system, and/or during generation of the first aerosol of the aerosol, whilst a user's mouth is located over the mouthpiece 33. This may thus allow the user to receive the active substance in their mouth, such as on at least one lip, or a tongue, of their mouth, such that the active substance may provide a physiological effect on the user of the aerosol provision system, and/or enhance a property; characteristic; flavour; and/or effect of the first aerosol, for example in some instances to act to enhance the flavour (and any related sensation) imparted on the user as part of the first aerosol.

It will be appreciated that where any such active substance is employed, that this in some embodiments may comprise any of a solid phase active substance; a liquid or gel phase active substance; or a gaseous phase active substance. In some embodiments, the flavouring material could equally comprise a gel phase active substance, or could comprise a gritty texture (e.g. from the combination of a liquid/gel phase active substance along with at least one solid phase active substance within the liquid/gel phase active substance). In some embodiments, a gas phase active substance is derivable from a liquid phase active substance contained within a reservoir.

In so far as any active substance is employed in the second reservoir 101, in some embodiments there may be provided at least one one-way mechanism 191, such as a one-way valve for allowing active substance to pass from the second reservoir 101 to the at least one outlet 60A, but not from the at least one outlet to the second reservoir 101.

As to how any active substance may be delivered to the at least one outlet in use, it will be appreciated that this may be effected either automatically by the aerosol provision system 1 in use, such as via an actuator (such as a piston) which may be configured to force/direct a portion of the active substance, and/or in response to a predetermined event occurring.

It is also envisaged, however, that in some embodiments the active substance may be delivered to the at least one outlet in response to a predetermined event which is configured to be effected by a mouth of a user from the aerosol provision system whilst the mouth is located over the mouthpiece 33. In this way, the user may then be able to decide, by implementing an action with their mouth, such as via a lip or a tongue of the mouth, when they would like to receive a portion of the active substance. An example of this may be where the active substance comprises a flavouring material, for example such as menthol, and/or a sensate comprising a cooling agent and/or a tingling agent. In this way, the user may be able to perform an appropriate action with their mouth on the mouthpiece to allow the active substance to be delivered to the user's mouth via the at least one outlet, to thus then provide a minty/cool/icy feeling in the user's mouth which the user may find pleasing, e.g. as part of an aerosol.

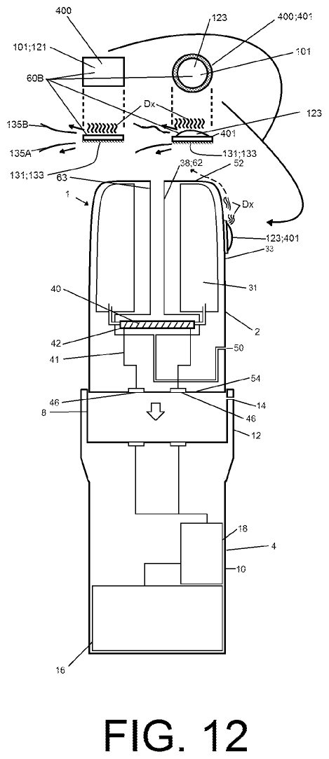

In terms of what such a predetermined event may comprise, it is envisaged that this in some embodiments may comprise the second reservoir 101 being at least partially compressed by the mouth of a user (e.g. using the user's teeth or a lip) to squeeze a portion of the active substance towards the at least one outlet (e.g. via an exposed, compressible, surface from the second reservoir 101 which is located flush with an outer surface of the mouthpiece 33, which is an exposed, compressible, surface which the user's mouth may compress in use). This might then act as a variant of the embodiment from FIG. 8, where the second reservoir then comprises a resilient/compressible surface, which may be flush with an outer surface of the mouthpiece 33, and which the user can then compress with their mouth, to squeeze a portion of the active substance towards the at least one outlet 60B. In such embodiments, the second reservoir 101 as noted above may be at least partially compressible and/or may be located in a cavity or recess from the mouthpiece 33 for receiving the second reservoir 101. In some of these embodiments, and then for allowing the second reservoir to be changed, the aerosol provision system 1 may comprise a consumable 400 (such as the consumable 400 types shown in the embodiments from FIGS. 12-14) which comprises the second reservoir 101, wherein the consumable 400 is detachable from the mouthpiece 33 and/or wherein the consumable 400 is configured to be received in any such employed cavity from the mouthpiece 33.

From the foregoing remarks therefore, it is appreciated that the present disclosure may also provide for embodiments as related to the numbered, non-limiting, paragraphs set out as follows:

1. An aerosol provision system for generating an aerosol, wherein the aerosol provision system comprises:
a mouthpiece comprising at least one outlet;
a first reservoir for storing aerosol-generating material, wherein the aerosol provision system is configured to generate an aerosol using the aerosol-generating material; and
a second reservoir comprising an active substance, which may optionally comprise a flavouring material;
wherein the aerosol provision system is configured to deliver the active substance to the at least one outlet of the mouthpiece in response to a predetermined event which is configured to be effected by a mouth of a user from the aerosol provision system whilst the mouth is located over the mouthpiece.

2. An aerosol provision according to paragraph 1, wherein the predetermined event is configured to be effected by a lip or tongue from the mouth of the user.

3. An aerosol provision according to paragraph 1 or 2, wherein the predetermined event is configured to be effected by the actuation of an actuator from the aerosol provision system using the mouth of a user.

4. An aerosol provision according to paragraph 3, wherein the mouthpiece comprises the actuator.

5. An aerosol provision according to any preceding paragraph, wherein the predetermined event comprises the second reservoir being at least partially compressed by the mouth of a user to squeeze a portion of the active substance towards the at least one outlet.

6. An aerosol provision according to any preceding paragraph, wherein the second reservoir is at least partially compressible.

7. An aerosol provision according to any preceding paragraph, wherein the second reservoir comprises an active substance transport element for storing the active substance in the second reservoir.

8. An aerosol provision according to any preceding paragraph, further comprising a one-way valve for allowing active substance to pass from the second reservoir to the at least one outlet, but not from the at least one outlet to the second reservoir.

9. An aerosol provision according to any preceding paragraph, wherein the aerosol provision system comprises a consumable which comprises the second reservoir, wherein the consumable is detachable from the mouthpiece.

10. An aerosol provision according to paragraph 9, wherein the mouthpiece comprises a cavity for receiving the consumable.

11. An aerosol provision according to any preceding paragraph, wherein the active substance comprises flavouring material, such as optionally menthol.

12. An aerosol provision according to any preceding paragraph, wherein the active substance comprises nicotine.

13. An aerosol provision according to any preceding paragraph, wherein the active substance comprises (or is) a solid phase active substance.

14. An aerosol provision according to any preceding paragraph, wherein the active substance comprises (or is) a liquid phase active substance.

15. An aerosol provision according to any preceding paragraph, wherein the active substance comprises (or is) a gaseous phase active substance.

16. An aerosol provision according to any preceding paragraph, wherein the active substance comprises (or is) a gel phase active substance.

17. A cartridge for an aerosol provision system for generating an aerosol, wherein the cartridge comprises:
a mouthpiece comprising at least one outlet;
a first reservoir for storing aerosol-generating material, wherein the aerosol provision system is configured to generate an aerosol using the aerosol-generating material; and
a second reservoir comprising an active substance;
wherein the cartridge is configured to deliver the active substance to the at least one outlet of the mouthpiece in response to a predetermined event which is configured to be effected by a mouth of a user from the aerosol provision system whilst the mouth is located over the mouthpiece.

18. A mouthpiece for an aerosol provision system for generating an aerosol, wherein the mouthpiece comprises:
at least one outlet;
a first reservoir for storing aerosol-generating material, wherein the aerosol provision system is configured to generate an aerosol using the aerosol-generating material; and
a second reservoir comprising an active substance;
wherein the mouthpiece is configured to deliver the active substance to the at least one outlet in response to a predetermined event which is configured to be effected by a mouth of a user from the aerosol provision system whilst the mouth is located over the mouthpiece.

19. A method of enhancing an aerosol delivered to a mouth of a user from an aerosol provision system, wherein the mouth is located over a mouthpiece from the aerosol provision system, wherein the method comprises:
generating the aerosol using aerosol-generating material from a first reservoir of the aerosol provision system;
delivering the aerosol to the mouth of the user whilst the mouth is located over the mouthpiece; and
delivering an active substance to the mouth of the user from a second reservoir of the aerosol provision system, which is separate from the first reservoir, whilst the mouth is located over the mouthpiece and whilst the aerosol is delivered to the mouth of the user.

20. A method according to claim 19, wherein the method further comprises:
effecting a predetermined event by a mouth of a user from the aerosol provision system whilst the mouth is located over the mouthpiece; and
delivering the active substance to the mouth of the user in response to the predetermined event being effected by the mouth of the user.

The above disclosure aside, the present disclosure also provides for the embodiments as illustrated in FIGS. 12-14, which relate to the usage of a consumable 400 for use with an aerosol provision system 1.

In these embodiments, there is thus provided, at a very general level, a consumable 400, for use in an aerosol provision system 1 for generating a first aerosol, wherein the consumable comprises a reservoir 101 for storing an active substance, wherein the consumable 400 comprises an outlet for delivering the active substance from the second reservoir to a user of the aerosol provision system.

Thus at a general level, it may be appreciated that the consumable 400 may be effectively retrofitted onto an existing aerosol provision system 1, such as in some narrower embodiments by being releasable engaged to, or attached to, a portion of the aerosol provision system 1. As to what such a portion might be, this inferably could be any appropriate part of the aerosol provision system 1 which may allow the active substance from the consumable 400 to be delivered to the user of the aerosol provision system 1. In this way, the consumable 400 may allow the user to be exposed to both the first aerosol from the aerosol provision system 1, and also the active substance from the reservoir 101 of the consumable 400.

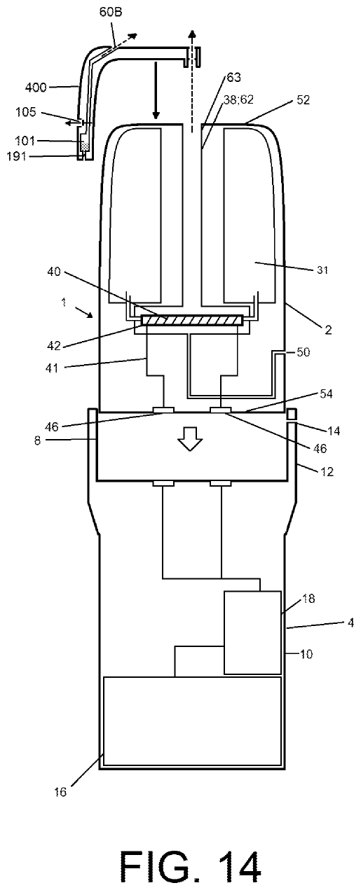

In some embodiments, such as those shown in FIGS. 12-14, it may be thus seen that the consumable 400 may be configured to releasably attach/engage a mouthpiece 33 from the aerosol provision system 1, and/or any employed cartridge 2 of the aerosol provision system 1. Any consumable 400 may equally be releasable attached/engageable with an outer surface of the aerosol provision system 1 (as shown in the embodiments of FIGS. 12-14, where this outer surface may be a surface of the mouthpiece 33, for example). Yet in some embodiments, as shown in FIG. 14 for instance, the consumable 400 may be releasably attached/engageable with an inner surface 63 of the aerosol provision system 1, such as by being releasably attached/engageable with the aerosol outlet tube 38;62, from the mouthpiece 33, which is configured to receive the first aerosol. This latter attachment/engagement has been found to be particularly beneficial in so far as it has been found to reduce the extent of re-condensing first aerosol which may otherwise form on the outer surfaces of the mouthpiece 33/aerosol provision system 1.

In so far as any consumable 400 may be employed, it may be appreciated that the consumable 400 may comprise the reservoir 101 for storing the active substance.

The reservoir 101 in some instances, along with comprising the active substance, could potentially also include a carrier constituent for the active substance. This might especially be so, where the active substance comprises a solid phase active substance, or non-flowing substance, for example. So in these instances, the carrier constituent can help to deliver the active substance from the reservoir 101 towards the user. Where such a carrier constituent is employed, this appreciably could comprise liquid components, preferably selected from: water; glycerine; propylene glycol and combinations thereof (e.g. as per some of the compositions described above, e.g. those described from Table 1 for example).

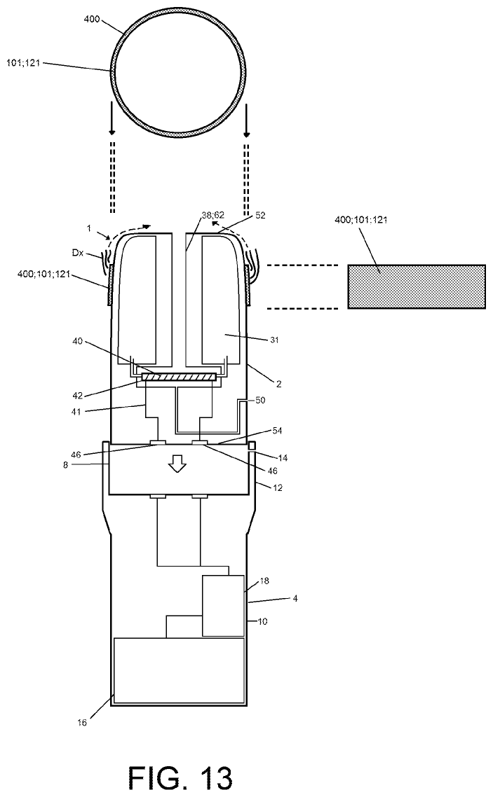

As to the nature of any employed reservoir 101 as well, appreciably this could be provided in a number of different ways, such as through the second reservoir 101 comprising a porous material 121 for storing the active substance. Such porous material 121 may then be configured to hold, or be soaked in, the active substance. The nature of this porous material appreciably could be any number of things, such as through the porous material 121 comprising paper; cardboard; or a foam, for example. In so far as paper might be employed, in some embodiments the paper may comprise rice paper for allowing the overall thickness of the porous material 121 to be reduced, and thus allow for a relatively high surface area/volume ratio for the second reservoir 101 as a whole. An example of such embodiments may be seen in the embodiment from FIG. 12 (see the top-left consumable example) and also FIG. 13, which each show there being provided a consumable 400 comprising cardboard (or potentially paper), whose second reservoir 101 is a portion of the cardboard (or paper) itself, which is porous and which is soaked in the active substance. In this way, the active substance may be delivered/atomised/vaporised from the cardboard to generate a second aerosol or a vapour, which can then diffuse toward the user for then mixing with the first aerosol passing out from the outlet 60 of the aerosol provision system 1, as shown by the diffusion areas ‘Dx’ in FIGS. 12 and 13.

With reference to any such porous material 121, it may been seen that this may be also more widely employed to provide a permeable portion, from the aerosol provision system (or consumable 400/second reservoir 101), which is configured to allow the active substance to be selectively released from the second reservoir 101 as air is supplied to the active substance from an air inlet from the aerosol provision system. Such an air inlet could, for instance, be an air inlet from the consumable, or could be used in some other embodiments, such as that shown in FIG. 8A where there is employed the second air inlet 50B. In this way, the permeable portion could for example comprise the porous material 121, in the second reservoir 101, which might allow any active substance located in this second reservoir 101 to be selectively released from the porous material 121 as air is supplied to the active substance from the second air inlet 50B.

An example application of the above embodiments may be thus to have the active substance from the consumable 400 as comprising a flavouring material, such as menthol. In this way, and to the extent that the user might then want a menthol flavouring, and/or an extra amount of nicotine, during usage of the first aerosol/aerosol provision system 1, the user can then selectively attach the consumable 400 to the aerosol provision system 1 such to then allow the user to bolster/enhance the first aerosol with the active substance comprising this extra flavouring material. In this way, the functionality of the original aerosol provision system 1 can be boosted/modified, as required, through appropriate usage of any required consumable 400 and its constituent active substance, which itself could be an aerosol-generating material/vapour-generating material in some embodiments, as will be appreciated.

In some embodiments, any employed consumable 400 could also be provided as part of a pod 401, or capsule 401, consumable, as shown in the embodiment from FIG. 12 (see the top-right consumable example). Where such a consumable is employed 400, the reservoir 101 therefrom may comprise a resilient outer shell 123 for storing the active substance. The resilient outer shell may then be partially deformable, even though it may be filled with active substance, in a conceptually similar arrangement to that of a detergent pod/capsule. In this respect, and in some embodiments, the resilient outer shell 123 may comprise a polymeric material, and/or could be transparent or translucent (or more generally the second reservoir 101 could be transparent or translucent). In this way, a user may then be able to identify a remaining amount of active substance in the reservoir, through this transparent or translucent portion.

Where such a consumable is employed, it may be appreciated that the resilient outer shell 123 may be made of a porous or breathable material, which may be permeable enough for vapour release, such to allow an aerosol/vapour to be released/diffused from the consumable 400. Tying in with this, greater release/diffusion of a second aerosol may be possible where a relatively thin resilient outer shell 123 is used, such as through the resilient outer shell comprising a maximum thickness of no more than 1 mm. By the term ‘maximum thickness’ here, this may be understood as meaning that no part of the resilient outer shell 123 has a thickness which is more than 1 mm. However, to provide an element of strength to the resilient outer shell 123, the resilient outer shell 123 may have a thickness which is more than 0.075 μm. Equally however, the consumable 400 may instead additionally/alternatively comprise at least one outlet 60B in communication with the reservoir 101 for allowing the active substance to be released from the consumable 400.

For allowing any employed consumable 400 to be more easily engaged/attached to the aerosol provision system 1, such as those shown in the embodiments of FIGS. 12-13, in some embodiments the consumable 400 may comprise an attachment means 131 for releasably attaching the consumable 400 to a portion, such as an external surface, and/or a mouthpiece 33, of the aerosol provision system 1. Any such attachment means 131 could inferably comprise any number of things, such as (but not limited to) any of: an adhesive; an adhesive patch; a resilient portion (for example along the lines of the attachment portion 109 shown in the embodiment from FIG. 9); a releasable latch (e.g. which might engage the recesses shown in the embodiment of FIG. 9, on the outer surface of the cartridge 2/aerosol provision system 1); and/or a fastener. The usage of an adhesive patch 133 is shown in the embodiments from FIG. 12 (see the top-left, and top-right, consumable examples), which may comprise an adhesive patch 133 for allowing the consumable 400 to be releasably attached to the outer surface of the aerosol provision system 1, such as the mouthpiece 33.

Related to the above features, it may be seen that in some instances thereof, the consumable 400 may be provided with a removable (potentially peelable) portion 135A, such as a portion defining a coating or film, which covers the attachment means 131. In this way, the removable portion 135A may be retained over the attachment means 131, until just prior to use of the consumable 400, where the removable portion 135A may then be removed to expose the attachment means 131 of the consumable 400. In this way, the removable portion 135A may help preserve the integrity (and/or any adhesiveness) of the attachment means 131.

Any provided consumable 400 may equally be provided with a removable (potentially peelable) portion 135B for inhibiting the release of any active substance from the consumable 400, in some embodiments—such as those shown in the embodiments from FIG. 12 (see the top-left, and top-right, consumable examples). In this way, and to the extent that the removable portion 135B in this instance may be sufficiently impermeable to vapour flow, this removable portion 135B may assist with preventing any vapour release/diffusion from the consumable 400 until when the consumable 400 is intended to be used.

That being said, the provision of such an impermeable removable portion 135B may not be necessary, for example to the extent that the consumable 400 might alternatively be retained in a sealed blister pack prior to use, which may otherwise assist with preventing any premature release of active substance from the consumable through not allowing any flow of air there-over, or there-past, which may otherwise assist with this active substance escapingfrom the consumable 400 in use.

Where any consumable 400, such as some those described above, is employed, it is envisaged that the consumable 400 may be configured to deliver its active substance without heating the active substance, or using a propellant, such as a compressed gas of gaseous propellant. In this way, the action of the user imparting an airflow through/over the consumable 400 (for example via any air inlet from the consumable—as shown in the embodiment from FIG. 14, or through any porous portion of the consumable 400 as per the cardboard consumable embodiments from FIGS. 12 and 13, or through the porous resilient outer shell 123 as per the second example from FIG. 12) may be enough to allow the active substance to be imparted, e.g. as part of an aerosol/vapour in some cases, in a direction towards the user during use. In so far as any provided consumable 400 does comprise an air inlet, however, as shown for example in the embodiment of FIG. 14, there may in some of these embodiments be also provided at least one one-way mechanism 191, such as a one-way valve, for allowing air to pass into the second reservoir 101, but not allow active substance to pass from the second reservoir 101 to the air inlet.

Any employed consumable 400 may also, in some embodiments, comprise the previously described throttling techniques for controlling the flow of material in/out of the consumable 400. In this way, and put differently, the consumable 400 may be configured to selectively prevent the delivery/escape of active substance from the reservoir, or may be configured to vary the rate at which the active substance is delivered from the reservoir. As to how this is achieved, this may appreciably be achieved through mechanical means and/or electrical means as required. For instance, a particular embodiment for varying the rate at which active substance may be delivered from the reservoir 101 towards the user may be through the consumable 400 comprising the throttling portion 105 (as shown in the embodiment from FIG. 14). With respect to this throttling portion 105 therefore, depending on its location, the throttling portion 105 may be configured to control the flow of air to the second reservoir 101, and/or control the flow of active substance from the second reservoir 101 towards the user and any provided outlet from the consumable 400. Where such a throttling portion 105 is provided, the throttling portion 105 could appreciably comprise a mechanical throttling portion, such as a moveable member inside the air/substance flow path relating to the second reservoir 101, such as being located just downstream of any provided air inlet of the consumable 400, and/or between the reservoir 101 and any provided outlet 60B from the consumable 400 (as shown in the embodiment from FIG. 14). The throttling portion 105 in some embodiments could also comprise a sharp bend, or portion of the consumable 400 which induces a change in direction, or perhaps even a vortex flow, inside the consumable 400.

From the foregoing disclosure, it will be appreciated that a number of different possibilities are described for augmenting an aerosol provision system, which generates a first aerosol using a first aerosol-generating material from a first reservoir, in a way which improves the characteristics or possibilities for the aerosol provision system as a whole. These possibilities could include the introduction of some of the herein described second aerosol techniques; and/or via some of the throttling techniques herein described; or via some of the described headspace techniques; or even via the introduction of the one or more active substances as herein described, from a second reservoir (which may in some narrower instances be provided as part of a consumable), which can be additionally delivered to the user of the aerosol provision system 1 during use.

To be clear, it will be appreciated that where any second reservoir 101 is provided, notwithstanding any other features of the second reservoir 101 which may be employed, the second reservoir 101 in any of these embodiments, as required, may be configured to not comprise any combination of (or all of) a vaporiser; a heater/heating element; and/or a propellant. Put differently, in accordance with some embodiments the second reservoir may not comprise:
a) a vaporiser;
b) a vaporiser or a propellant;
c) a heater/heating element; and/or
d) a heater/heating element or a propellant.

Staying with any employed second reservoir 101, the second reservoir in some embodiments may not be maintained under reduced pressure.

It is also recognised that any employed second reservoir 101, as herein described, may also in some embodiments (such as those shown in the embodiments from the Figures, for example) not comprise a spring-loaded syringe.

It may also be seen that in accordance with some embodiments (such as, by way of a mere example, those relating to any employed consumable 400 as herein described) that any active substance in some of these embodiments may not be delivered from the second reservoir 101 to a user of the aerosol provision system 1 through an atomizing spray nozzle.

With respect to the first reservoir as well, for completeness, it may be seen that in some embodiments, any such first reservoir may be configured to not comprise a vaporiser; and/or a heater/heating element. And again, additionally/alternatively, in some embodiments, the first reservoir may not comprise a propellant.

In some embodiments, it is also envisaged in some instances, as required, that:
a) the first reservoir comprises a vaporiser which is a heater/heating element and the second reservoir does not comprise a vaporiser;
b) the first reservoir comprises a vaporiser which is a heater/heating element and the second reservoir does not comprise a vaporiser or a propellant;
c) the first reservoir comprises a vaporiser which is a heater/heating element and the second reservoir does not comprise a heater/heating element; and/or
d) the first reservoir comprises a vaporiser which is a heater/heating element and the second reservoir does not comprise a heater/heating element or a propellant.

In so far as both a first reservoir and a second reservoir (which for completeness is understood as also including any reservoir from any employed consumable 400) is employed to deliver nicotine to a user of the aerosol provision system 1, it may be seen that in some embodiments, it may be that the first reservoir comprises nicotine, but the second reservoir does not comprise nicotine. This could have the advantage in some cases of preventing a user from boosting the nicotine content deliverable from an existing aerosol provision system, via an added second reservoir which contains yet more nicotine, to an undesirable level. This being said, there may be other embodiments where nicotine, in some amount (which may not be the same as any nicotine amount, if any, which is present in the first reservoir), is employed as part of any provided second reservoir 101 (or reservoir from any employed consumable 400).

Appreciably though, and in accordance with some embodiments, any employed first and second reservoirs may comprise nicotine, wherein the concentration of nicotine contained within the second reservoir is greater than that contained in the first reservoir. Equally, in some embodiments, any employed first and second may comprise nicotine, wherein the concentration of nicotine contained within the first reservoir is greater than that contained in the second reservoir 101.

Where any combination of flavouring material(s) and sensate(s) are employed, in accordance with some specific embodiments, it may be the case that the first reservoir comprises one or more flavouring material(s) with any employed second reservoir 101 then comprising one or more sensate(s). In some other cases, it may be instead that the first reservoir comprises one or more sensate(s) and any employed second reservoir 101 comprises one or more flavouring material(s).

Related to this, and in some embodiments, it may be the case that the first and second reservoirs comprise one or more flavouring material(s), wherein the concentration of the one or more flavouring material(s) contained within the second reservoir 101 is greater than that contained in the first reservoir. Similarly, in some embodiments, the first and second reservoirs may comprise one or more flavouring material(s), wherein the concentration of the one or more flavouring material(s) contained within the first reservoir is greater than that contained in the second reservoir 101.

Thus from the foregoing, it may be seen that a plethora of different combinations exist as to what is contained in the first reservoir alongside what is contained in any employed second reservoir 101 (or reservoir from any employed consumable 400, which may be also be construed as operating in a related way to such a second reservoir). This thus provides a great degree of flexibility in the design/configuration of the aerosol provision system as a whole, as is exemplified at least by the various possibilities of features as outlined in the various set of clauses at the end of this specification. For the avoidance of any doubt, it will be commensurately appreciated that any features from these clauses may be combined as required in any combination beyond those as expressly set out in these clauses, noting the great flexibility and interchangeability in the usage of such features which the present disclosure clearly provides for.

### Consistory Set of Clauses

1. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing an active substance.

2. An aerosol provision system according to clause 1, wherein the aerosol provision system comprises an outlet for delivering the active substance from the second reservoir to a user of the aerosol provision system.

3. An aerosol provision system according to any preceding clause, wherein the active substance comprises a second aerosol-generating material, wherein the aerosol provision system is configured to generate a second aerosol using the second aerosol-generating material.

4. An aerosol provision system according to clause 3, wherein the aerosol provision system is configured to generate the first aerosol independently of the second aerosol.

5. An aerosol provision system according to any preceding clause, further comprising an aerosol generator for generating the first aerosol.

6. An aerosol provision system according to clause 5, wherein the aerosol generator comprises a heating element.

7. An aerosol provision system according to any preceding clause, when further dependent on clause 3 at least, wherein the aerosol provision system is configured to generate the first aerosol at a first temperature, and is configured to generate the second aerosol at a second temperature, wherein the first temperature is greater than the second temperature.

8. An aerosol provision system according to any preceding clause, when further dependent on clause 3 at least, wherein the aerosol provision system is configured to generate the second aerosol at a temperature of no more than 50 degree Celsius.

9. An aerosol provision system according to any preceding clause, when further dependent on clause 3 at least, wherein the aerosol provision system is configured to generate the second aerosol at a temperature of no more than 40 degree Celsius.

10. An aerosol provision system according to any preceding clause, when further dependent on clause 3 at least, wherein the aerosol provision system is configured to generate the second aerosol without actively heating the second aerosol generating material.

11. An aerosol provision system according to any preceding clause, wherein the aerosol provision system is configured to deliver the active substance without actively heating the active substance.

12. An aerosol provision system according to any preceding clause, wherein the second reservoir is configured to be at least partially heated, and/or at least passively heated, by the first aerosol.

13. An aerosol provision system according to any preceding clause, wherein the second reservoir is configured to be heated during delivery of the active substance.

14. An aerosol provision system according to any preceding clause, wherein the second reservoir is configured to not be heated during delivery of the active substance.

15. An aerosol provision system according to any preceding clause, wherein the second reservoir is configured to be cooled during delivery of the active substance.

16. An aerosol provision system according to any preceding clause, wherein the first reservoir is configured to be not be heated during generation of the first aerosol.

17. An aerosol provision system according to any preceding clause, wherein the first reservoir is configured to be cooled during generation of the first aerosol.

18. An aerosol provision system according to any preceding clause, wherein the first reservoir is configured to be heated during generation of the first aerosol.

19. An aerosol provision system according to any preceding clause, wherein the aerosol provision system is configured to generate the first aerosol at a temperature of at least 15 degrees Celsius.

20. An aerosol provision system according to any preceding clause, wherein the aerosol provision system is configured to deliver the active substance downstream of the first aerosol.

21. An aerosol provision system according to any preceding clause, wherein the second reservoir is downstream of the first reservoir.

22. An aerosol provision system according to any preceding clause, wherein the aerosol provision system is configured to deliver the active substance upstream of the first aerosol.

23. An aerosol provision system according to any preceding clause, wherein the aerosol provision system is configured to deliver the active substance the active substance into the first reservoir.

24. An aerosol provision system according to any preceding clause, wherein the second reservoir is upstream of the first reservoir.

25. An aerosol provision system according to any preceding clause, wherein the aerosol provision system is configured to supply the first aerosol through the second reservoir.

26. An aerosol provision system according to any preceding clause, wherein the aerosol provision system is configured to deliver the active substance through the first reservoir.

27. An aerosol provision system according to any preceding clause, further comprising an aerosol outlet channel for receiving the first aerosol.

28. An aerosol provision system according to clause 27, wherein the aerosol outlet channel comprises an aerosol outlet tube.

29. An aerosol provision system according to clause 27 or 28, wherein the aerosol provision system is configured to supply the active substance into the aerosol outlet channel.

30. An aerosol provision system according to clause 29, wherein the aerosol provision system is configured to supply the active substance into the aerosol outlet channel via at least one opening in the aerosol outlet channel.

31. An aerosol provision system according to clause 30, wherein the at least one opening comprises a plurality of openings.

32. An aerosol provision system according to clause 30 or 31, wherein each opening comprises a cross-sectional area of no more than 25 mm².

33. An aerosol provision system according to any of clauses 30-32, wherein each opening comprises a cross-sectional area of no more than 15 mm².

34 An aerosol provision system according to any of clauses 27-33, wherein the aerosol outlet channel is configured to supply the first aerosol to a first aerosol outlet from the aerosol provision system.

35. An aerosol provision system according to clause 34, when further dependent on clause 30 at least, wherein each opening is located upstream of the first aerosol outlet.

36. An aerosol provision system according to clause 34 or 35, wherein the aerosol provision system is configured to supply the active substance into the aerosol outlet channel in a direction which extends towards the first aerosol outlet.

37. An aerosol provision system according to any of clauses 27-36, when further dependent on clause 5 at least, wherein the aerosol provision system is configured to supply the active substance into the aerosol outlet channel in a direction which extends away from the aerosol generator.

38. An aerosol provision system according to any preceding clause, when dependent on clause 27 at least, wherein the aerosol provision system is configured to supply the active substance around the aerosol outlet channel.

39. An aerosol provision system according to any preceding clause, when further dependent clauses 2 and 34 at least, wherein the outlet is separate from the first aerosol outlet.

40 An aerosol provision system according to clause 39, wherein the outlet at least partially surrounds the first aerosol outlet.

41. An aerosol provision system according to any preceding clause, when further dependent on clause 2 at least wherein the outlet is annular.

42. An aerosol provision system according to any preceding clause, when further dependent on clauses 2 and 34 at least, wherein the first aerosol outlet is downstream of the outlet.

43. An aerosol provision system according to any preceding clause, when further dependent on clauses 2 and 30 at least, wherein the outlet comprises the at least one opening in the aerosol outlet channel.

44. An aerosol provision system according to any preceding clause, wherein the aerosol provision system is configured to generate the first aerosol using air supplied from a first air inlet from the aerosol provision system.

45. An aerosol provision system according to any preceding clause, wherein the aerosol provision system is configured to deliver the active substance using air supplied from a second air inlet from the aerosol provision system.

46. An aerosol provision system according to clause 45, when further dependent on clause 44, wherein the first air inlet is separate from the second air inlet.

47. An aerosol provision system according to any preceding clause, further comprising a mouthpiece.

48. An aerosol provision system according to clause 47, when further dependent on clause 46, wherein the second air inlet is located more proximal to the mouthpiece than the first air inlet is located to the mouthpiece.

49. An aerosol provision system according to clause 47 or 48, wherein the second reservoir is configured to engage the mouthpiece.

50. An aerosol provision system according to clause 49, wherein the second reservoir is configured to engage an outer surface of the mouthpiece.

51. An aerosol provision system according to any of clauses 47-50, wherein the second reservoir is configured to operate as a sleeve which can engage around the mouthpiece.

52. An aerosol provision system according to any of clauses 47-51, wherein the second reservoir is configured to be located within a recess of the mouthpiece, wherein the recess is shaped to accommodate at least a portion of the second reservoir.

53. An aerosol provision system according to clause 52, wherein the recess is shaped to accommodate the entirety of the second reservoir.

54. An aerosol provision system according to any preceding clause, wherein the second reservoir comprises a compressible portion, wherein the compressible portion is configured such that compression of the compressible portion is configured to allow the active substance to be released from the second reservoir.

55. An aerosol provision system according to any preceding clause, wherein the second reservoir is a consumable.

56. An aerosol provision system according to any preceding clause, wherein the second reservoir comprises an attachment means for releasably attaching the second reservoir to a portion of the aerosol provision system.

57. An aerosol provision system according to clause 56, when further dependent on clause 47 at least, wherein the portion comprises the mouthpiece.

58. An aerosol provision system according to clause 56 or 57, wherein the portion comprises an aerosol outlet tube, from the mouthpiece, which is configured to receive the first aerosol.

59. An aerosol provision system according to any of clauses 56-58, wherein the attachment means comprises an adhesive.

60. An aerosol provision system according to any of clauses 56-59, wherein the attachment means comprises an adhesive patch.

61. An aerosol provision system according to any of clauses 56-50, wherein the attachment means comprises a resilient portion.

62. An aerosol provision system according to any of clauses 56-61, wherein the attachment means comprises a releasable latch.

63. An aerosol provision system according to any of clauses 56-62, wherein the attachment means comprises a fastener.

64. An aerosol provision system according to any preceding clause, further comprising the first aerosol-generating material in the first reservoir.

65. An aerosol provision system according to clause 64, wherein the first aerosol-generating material comprises a flavouring material.

66. An aerosol provision system according to clause 65, wherein the flavouring material comprises at least one of menthol; tobacco.

67. An aerosol provision system according to any preceding clause, wherein the first aerosol-generating material comprises a medicament.

68. An aerosol provision system according to any preceding clause, further comprising the active substance in the second reservoir.

69. An aerosol provision system according to any preceding clause, wherein the active substance comprises a solid phase active substance.

68. An aerosol provision system according to any preceding clause, wherein the active substance comprises a liquid phase active substance.

69. An aerosol provision system according to any preceding clause, wherein the active substance comprises a gel phase active substance.

70. An aerosol provision system according to any preceding clause, wherein the active substance comprises a gas phase active substance.

71. An aerosol provision system according to any preceding clause, wherein the active substance comprises nicotine, a flavouring material, a sensate material, or a combination thereof.

72. An aerosol provision system according to clause 71, wherein the flavouring material comprises at least one of menthol; and/or tobacco.

73. An aerosol provision system according to any preceding clause, wherein the the active substance comprises a medicament.

74. An aerosol provision system according to any preceding clause, wherein the aerosol provision system is configured to generate the first aerosol simultaneously to delivering the active substance.

75. An aerosol provision system according to any preceding clause, wherein the aerosol provision system is configured to mix the active substance with the first aerosol.

76. An aerosol provision system according to clause 75, wherein the aerosol provision system is configured to supply the mixed first aerosol and active substance to a first aerosol outlet from the aerosol provision system.

77. An aerosol provision system according to any preceding clause, when further dependent on clause 27 at least, wherein the second reservoir at least partially surrounds the aerosol outlet channel.

78. An aerosol provision system according to any preceding clause, when further dependent on clause 28, wherein the second reservoir is configured to surround the aerosol outlet tube.

79. An aerosol provision system according to any preceding clause, wherein the second reservoir is annular.

80. An aerosol provision system according to any preceding clause, wherein the second reservoir at least partially surrounds the first reservoir.

81. An aerosol provision system according to any preceding clause, wherein the first reservoir at least partially surrounds the second reservoir.

82. An aerosol provision system according to any preceding clause, wherein the first reservoir comprises a volumetric capacity of no more than 50 ml.

83. An aerosol provision system according to any preceding clause, wherein the first reservoir comprises a volumetric capacity of no more than 25 ml.

84. An aerosol provision system according to any preceding clause, wherein the second reservoir comprises a volumetric capacity of no more than 50 ml.

85. An aerosol provision system according to any preceding clause, wherein the second reservoir comprises a volumetric capacity of no more than 25 ml.

86. An aerosol provision system according to any preceding clause, when further dependent on clause 3 at least, wherein the aerosol provision system is configured for allowing the rate of generation of the second aerosol to be varied.

87. An aerosol provision system according to any preceding clause, when further dependent on clause 3 at least, wherein the aerosol provision system further comprises a throttling portion for varying at least one of the rate of generation of the second aerosol and the rate at which the second aerosol is configured to exit the aerosol provision system.

88. An aerosol provision system according to clause 87, wherein the throttling portion comprises a mechanical throttling portion.

89. An aerosol provision system according to clause 87 or 88, when further dependent on clause 27, wherein the throttling portion is located outside of the aerosol outlet channel.

90. An aerosol provision system according to any of clauses 87-89, when further dependent on clause 30, wherein the throttling portion is located upstream of the at least one opening.

91. An aerosol provision system according to any of clauses 87-90, when further dependent on clause 45, wherein the second air inlet comprises an adjustable size, and the throttling portion comprises the second air inlet.

92. An aerosol provision system according to any of clauses 87-91, wherein the throttling portion is configured to control a flow rate of air which is delivered to the second reservoir.

93. An aerosol provision system according to any of clauses 87-92, wherein the throttling portion comprises a variable sized orifice from the aerosol provision system.

94. An aerosol provision system according to any of clauses 87-93, wherein the throttling portion is user adjustable.

95. An aerosol provision system according to any preceding clause, wherein the aerosol provision system further comprises a cartridge and an aerosol provision device, wherein the aerosol provision device comprises a cartridge receiving section that includes an interface arranged to cooperatively engage with an interface from the cartridge so as to releasably couple the cartridge to the aerosol provision device.

96. An aerosol provision system according to clause 95, when further dependent on clause 5 at least, wherein the aerosol provision device further comprises a power supply and control circuitry configured to selective supply power from the power supply to the first aerosol generator from the cartridge.

97. An aerosol provision system according to any of clauses 95-96, wherein the cartridge comprises the first reservoir.

98. An aerosol provision system according to any of clauses 95-97, wherein the cartridge comprises the second reservoir.

99. An aerosol provision system according to any of clauses 95-98, when further dependent on clause 5, wherein the cartridge comprises the first aerosol generator.

100. An aerosol provision system according to any of clauses 95-99, when further dependent on clause 27, wherein the cartridge comprises the aerosol outlet channel.

101. An aerosol provision system according to any preceding clause, wherein the aerosol provision system further comprises at least one of a module, or consumable, which comprises the second reservoir.

102. An aerosol provision system according to clause 101, wherein the module/consumable is configured to be separable from the first reservoir.

103. An aerosol provision system according to clause 101 or 102, wherein the module/consumable is configured to receive the first aerosol.

104. An aerosol provision system according to any of clauses 101-103, wherein the active substance is configured to be located inside the module/consumable.

105. An aerosol provision system according to any of clauses 101-104, wherein the active substance is configured to mix with the first aerosol inside the module/consumable.

106. An aerosol provision system according to any of clauses 101-105, wherein the active substance is configured to mix with the first aerosol outside the module/consumable.

107. An aerosol provision system according to any of clauses 101-106, wherein the module/consumable comprises a mouthpiece.

108. An aerosol provision system according to any of clauses 101-107, when further dependent on clause 95, wherein the module/consumable is configured to releasably couple with the cartridge.

109. An aerosol provision system according to clause 108, wherein the cartridge comprises a mouthpiece, and the module/consumable comprises a second cartridge.

110. An aerosol provision system according to clause 108 or 109, wherein the module/consumable is configured to releasably couple over, or to, a mouthpiece from the cartridge for releasably coupling the module/consumable to the cartridge.

111. An aerosol provision system according to any preceding clause, wherein the first aerosol generating material comprises one or more sensates.

112. An aerosol provision system according to any preceding clause, wherein the first aerosol comprises one or more sensates.

113. An aerosol provision system according to any preceding clause, wherein the active substance comprises one or more sensates.

114. An aerosol provision system according to any preceding clause, when further dependent on clause 3 at least, wherein the second aerosol comprises one or more sensates.

115. An aerosol provision system according to any of clauses 111-114, wherein the one or more sensates is in an amount of 0.01 to 12% w/w.

116. An aerosol provision system according to any of clauses 112-115, wherein the one or more sensates comprises one or more cooling agent(s), one or more warming agents(s); and/or one or more tingling agent(s).

117. An aerosol provision system according to any preceding clause, wherein the first aerosol generating material comprises one or more acids.

118. An aerosol provision system according to any preceding clause, wherein the first aerosol comprises one or more acids.

119. An aerosol provision system according to any preceding clause, wherein the active substance comprises one or more acids.

120. An aerosol provision system according to any preceding clause, when further dependent on clause 3 at least, wherein the second aerosol comprises one or more acids.

121. An aerosol provision system according to any preceding clause, wherein the first aerosol generating material comprises one or more flavouring materials.

122. An aerosol provision system according to any preceding clause, wherein the first aerosol comprises one or more flavouring materials.

123. An aerosol provision system according to any preceding clause, wherein the active substance comprises one or more flavouring materials.

124. An aerosol provision system according to any preceding clause, when further dependent on clause 3 at least, further comprising a third reservoir for storing a third aerosol-generating material, wherein the aerosol provision system is configured to generate a third aerosol using the second aerosol-generating material.

125. An aerosol provision system according to clause 124, wherein the aerosol provision system is configured to generate the third aerosol independently of the first aerosol.

126. An aerosol provision system according to clause 124 or 125, wherein the aerosol provision system is configured to generate the third aerosol independently of the second aerosol.

127. An aerosol provision system according to any of clauses 124-126, wherein the aerosol provision system is configured to not generate the third aerosol at the same time as the second aerosol.

128. An aerosol provision system according to any of clauses 124-127, wherein the aerosol provision system is configured to prevent the second aerosol from being generated at the same time as the third aerosol is generated.

129. An aerosol provision system according to any of clauses 124-128, wherein the aerosol provision system is configured to operate in:
a first mode of operation in which the aerosol provision system is configured to generate the first aerosol and the second aerosol; and
a second mode of operation in which the aerosol provision system is configured to generate the first aerosol and the third aerosol.

130. An aerosol provision system according to clause 129, wherein the first mode of operation, the aerosol provision system is configured to not generate the third aerosol.

131. An aerosol provision system according to clause 129 or 130, wherein the second mode of operation, the aerosol provision system is configured to not generate the second aerosol.

132. An aerosol provision system according to any preceding clause, wherein the first reservoir comprises at least one headspace which is no smaller than 10% of the total volumetric capacity of the first reservoir.

133. An aerosol provision system according to any preceding clause, wherein the second reservoir comprises at least one headspace which is no smaller than 10% of the total volumetric capacity of the second reservoir.

134. An aerosol provision system according to any preceding clause, when further dependent on clause 124 at least, wherein the third reservoir comprises at least one headspace which is no smaller than 10% of the total volumetric capacity of the second reservoir.

135. An aerosol provision system according to any preceding clause, wherein the first reservoir comprises at least 10% carbon dioxide by volume.

136. An aerosol provision system according to any preceding clause, wherein the second reservoir comprises at least 10% carbon dioxide by volume.

137. An aerosol provision system according to any preceding clause, when further dependent on clause 124 at least, wherein the third reservoir comprises at least 10% carbon dioxide by volume.

138. An aerosol provision system according to any preceding clause, wherein either the first reservoir or the second reservoir comprises at least 9% carbon dioxide by volume; or wherein either the first reservoir or the second reservoir comprises at least 8% carbon dioxide by volume; or wherein either the first reservoir or the second reservoir comprises at least 7% carbon dioxide by volume; or wherein either the first reservoir or the second reservoir comprises at least 6% carbon dioxide by volume; or wherein either the first reservoir or the second reservoir comprises at least 5% carbon dioxide by volume; or wherein either the first reservoir or the second reservoir comprises at least 4% carbon dioxide by volume; or wherein either the first reservoir or the second reservoir comprises at least 3% carbon dioxide by volume; or wherein either the first reservoir or the second reservoir comprises at least 2% carbon dioxide by volume; or wherein either the first reservoir or the second reservoir comprises at least 1% carbon dioxide by volume.

139. An aerosol provision system according to any preceding clause, wherein the first aerosol and/or the first aerosol generating material comprises one or more cannabinoids.

140. An aerosol provision system according to any preceding clause, wherein the active substance comprises one or more cannabinoids.

141. An aerosol provision system according to any preceding clause, when further dependent on clause 124 at least, wherein the third aerosol and/or the third aerosol generating material comprises one or more cannabinoids.

142. An aerosol provision system according to any of clauses 139-141, wherein the one or more cannabinoids are configured to be heated.

143. An aerosol provision system according to any preceding clause, wherein the first aerosol and/or the first aerosol generating material is customisable.

144. An aerosol provision system according to any preceding clause, wherein the active substance, or the second aerosol and/or the second aerosol generating material when further dependent on clause 3, is customisable.

145. An aerosol provision system according to any preceding clause, when further dependent on clause 124 at least, wherein the third aerosol and/or the third aerosol generating material is customisable.

146. An aerosol provision system according to any preceding clause, wherein the active substance, or the second aerosol and/or the second aerosol generating material when further dependent on clause 3, is configured to be customisable using the aerosol provision system.

147. An aerosol provision system according to any preceding clause, when further dependent on clause 124 at least, wherein the third aerosol and/or the third aerosol generating material is configured to be customisable using the aerosol provision system.

148. An aerosol provision system according to any preceding clause, wherein the composition of at least one of the aerosols is configured to be customisable using the aerosol provision system.

149. An aerosol provision system according to preceding clause, wherein the flow rate of at least one of the aerosols is configured to be customisable using the aerosol provision system.

150. An aerosol provision system according to any preceding clause, wherein the flow rate of air which is operable to be delivered to at least one of the aerosol generating materials is configured to be customisable using the aerosol provision system.

151. An aerosol provision system according to any preceding clause, wherein the flow rate of air which is operable to be delivered to at least one of the aerosol generating materials is configured to be customisable using the aerosol provision system.

152. An aerosol provision system according to any preceding clause, wherein the first aerosol generating material or the active substance, or the second aerosol generating material when further dependent on clause 3 at least, comprises water.

153. An aerosol provision system according to any preceding clause, wherein the first aerosol generating material or the active substance, or the second aerosol generating material when further dependent on clause 3 at least, comprises no more than 20% w/w water.

154. An aerosol provision system according to any preceding clause, wherein the first aerosol generating material or the active substance, or the second aerosol generating material when further dependent on clause 3 at least, comprises no more than 80% w/w glycerine.

155. An aerosol provision system according to any preceding clause, wherein the first aerosol generating material or the active substance, or the second aerosol generating material when further dependent on clause 3 at least, comprises no more than 75% w/w propylene glycol.

156. An aerosol provision system according to any preceding clause, wherein the first aerosol generating material or the active substance, or the second aerosol generating material when further dependent on clause 3 at least, comprises propylene glycol.

157. An aerosol provision system according to clause 156, wherein the first aerosol generating material or the active substance, or the second aerosol generating material when further dependent on clause 3 at least, comprises no more than 75% propylene glycol.

158. An aerosol provision system according to any preceding clause, wherein the second reservoir comprises paper, wherein the paper is configured for storing the active substance.

159. An aerosol provision system according to clause 158, wherein the paper comprises rice paper.

160. An aerosol provision system according to any preceding clause, wherein the second reservoir comprises cardboard, wherein the cardboard is configured for storing the active substance.

161. An aerosol provision system according to clause 160, wherein the cardboard comprises a maximum thickness of no more than 10 mm.

162. An aerosol provision system according to clause 160 or 161, wherein the cardboard comprises a sheet of cardboard.

163. An aerosol provision system according to any preceding clause, when further dependent on clause 47 at least, when the second reservoir is shaped to match an outer surface of the mouthpiece.

164. An aerosol provision system according to any preceding clause, wherein the second reservoir comprises a resilient outer shell for storing the active substance.

165. An aerosol provision system according to clause 164, wherein the resilient outer shell comprises a polymeric material.

166. An aerosol provision system according to clause 164 or 165, wherein the resilient outer shell comprises a maximum thickness of no more than 1 mm.

167. An aerosol provision system according to any of clauses 164-167, when further dependent on clause 54 at least, wherein the attachment means is affixed to the resilient outer shell.

168. An aerosol provision system according to any preceding clause, wherein the second reservoir comprises a porous material for storing the active substance.

169. An aerosol provision system according to clause 168, wherein the porous material is configured to hold, or be soaked in, the active substance.

170. An aerosol provision system according to clause 168 or 169, wherein the porous material comprises paper; cardboard; or a foam.

171 An aerosol provision system according to any preceding clause, wherein the second reservoir is configured to be moved from a first position in which:
the active substance cannot be delivered to the outlet; and/or
the active substance cannot exit from the second reservoir.

172 An aerosol provision system according to any preceding clause, when further dependent on clause 3 at least, wherein the second reservoir is configured to be moved from a first position in which:
the second aerosol cannot be generated; and/or
the second aerosol-generating material cannot exit from the second reservoir.

173. An aerosol provision system according to any preceding clause, wherein the second reservoir is configured to be moved to a second position in which:
the active substance can be delivered to the outlet; and/or
the active substance can exit from the second reservoir.

174 An aerosol provision system according to any preceding clause, when further dependent on clause 3 at least, wherein the second reservoir is configured to be moved to a second position in which:
the second aerosol can be generated; and/or
the second aerosol-generating material can exit from the second reservoir.

175. An aerosol provision system according to clause 173 or 174, when further dependent on clause 171 or 172, wherein the second reservoir is configured to be moved between the first position and the second position.

176. An aerosol provision system according to clause 175, wherein the second reservoir is configured to rotate between the first position and the second position.

177. An aerosol provision system according to clause 175, wherein the second reservoir is configured to twist between the first position and the second position.

178. An aerosol provision system according to clause 175, wherein the second reservoir is configured to translate between the first position and the second position.

179. An aerosol provision system according to clause 175, wherein the second reservoir is configured to slide between the first position and the second position.

180. An aerosol provision system according to any preceding clause, wherein the second reservoir is annular.

181. An aerosol provision system according to any preceding clause, wherein the aerosol provision system is configured to deliver the active substance from the second reservoir without heating the second reservoir.

182. An aerosol provision system according to any preceding clause, when further dependent on clause 3 at least, wherein the aerosol provision system is configured to generate the second aerosol using the second aerosol-generating material without heating the second reservoir.

183. A consumable, for use in an aerosol provision system for generating an aerosol, wherein the consumable comprises:
a first reservoir for storing a first aerosol-generating material, wherein the aerosol provision system is configured to generate a first aerosol using the first aerosol-generating material; and
a second reservoir for storing an active substance.

184. A consumable according to clause 183, wherein the consumable comprises an outlet for delivering the active substance from the second reservoir to a user of the aerosol provision system.

185. A consumable according to clause 183 or 184, wherein the active substance comprises a second aerosol-generating material, wherein the consumable is configured to generate a second aerosol using the second aerosol-generating material.

186. A consumable according to clause 185, wherein the aerosol provision system is configured to generate the first aerosol independently of the second aerosol.

187. A consumable for use in an aerosol provision system for generating an aerosol, wherein the consumable comprises:
a first reservoir for storing a first aerosol-generating material, wherein the aerosol provision system is configured to generate a first aerosol using the first aerosol-generating material;
a second reservoir for storing a second aerosol-generating material, wherein the consumable is configured to generate a second aerosol using the second aerosol-generating material.

188. A consumable according to clause 187, wherein the consumable is configured to generate the first aerosol independently of the second aerosol.

189. A consumable for use in an aerosol provision system for generating a first aerosol, wherein the consumable comprises a reservoir for storing an active substance.

190. A consumable according to clause 189, wherein the consumable comprises an outlet for delivering the active substance from the reservoir to a user of the aerosol provision system.

191. A consumable according to clause 189 or 190, wherein the active substance comprises a second aerosol-generating material, wherein the consumable is configured to generate a second aerosol using the second aerosol-generating material.

192. A consumable according to clause 191, wherein the consumable is configured to generate the second aerosol without heating the second aerosol-generating material.

193. A consumable according to any of clauses 189-192, wherein the consumable is configured to deliver the active substance from the reservoir to a user of the aerosol provision system without heating the active substance.

194. A consumable according to any of 189-193, wherein the consumable is configured for allowing the active substance to be released from the reservoir through at least one outlet of the consumable.

195. A consumable according to any of clauses 189-194, wherein the reservoir comprises a compressible portion, wherein the compressible portion is configured such that compression of the compressible portion is configured to allow the active substnace to be released from the reservoir.

196. A consumable according to clause any of clauses 189-195, wherein the consumable comprises an attachment means for releasably attaching the consumable to a portion of the aerosol provision system.

197. A consumable according to clause 196, wherein the attachment means comprises an adhesive.

198. A consumable according to any of clauses 196-197, wherein the attachment means comprises an adhesive patch.

199. A consumable according to any of clauses 196-198, wherein the attachment means comprises a resilient portion.

200. A consumable according to any of clauses 196-199, wherein the attachment means comprises a releasable latch.

201. A consumable according to any of clauses 196-200, wherein the attachment means comprises a fastener.

202. A consumable according to any of clauses 189-201, wherein the reservoir comprises paper, wherein the paper is configured for storing the active substance.

203. A consumable according to clause 202, wherein the paper comprises rice paper.

204. A consumable according to any of clauses 189-203, wherein the reservoir comprises cardboard, wherein the cardboard is configured for storing the active substance.

205. A consumable according to any clause 204, wherein the cardboard comprises a maximum thickness of no more than 10 mm.

206. A consumable according to any of clauses 204-205, wherein the cardboard comprises a sheet of cardboard.

207. A consumable according to any of clauses 189-206, wherein the reservoir comprises a resilient outer shell for storing the active substance.

208. A consumable according to clause 207, wherein the resilient outer shell comprises a polymeric material.

209. A consumable according to any of clauses 207-208, wherein the resilient outer shell comprises a maximum thickness of no more than 1 mm.

210. A consumable according to any of clauses 207-209, when further dependent on clause 196 at least, wherein the attachment means is affixed to the resilient outer shell.

211. A consumable according to any of clauses 189-210, wherein the reservoir comprises a porous material for storing the active substance.

212. A consumable according to clause 211, wherein the active substance is configured to hold, or be soaked in, the porous material.

213. A consumable according to any of clauses 211-212, wherein the porous material comprises paper; cardboard; or a foam.

214. A consumable according to any of clauses 189-213, further comprising the active substance in the reservoir.

215. A consumable according to any of clauses 189-214, wherein the active substance comprises a solid phase active substance.

216. A consumable according to any of clauses 189-215, wherein the active substance comprises a liquid and/or gel phase active substance.

217. A consumable according to any of clauses 189-216, wherein the active substance comprises a gas phase active substance.

218. A consumable according to any of clauses 189-217, wherein active substance comprises nicotine and/or a flavouring material.

219. A consumable according to clause 218, wherein the flavouring material comprises at least one of menthol; and/or tobacco.

220. A consumable according to any of clauses 189-219, wherein the active substance comprises a medicament.

221. An assembly comprising a consumable according to any of clauses 187-220 and the aerosol provision system.

222. An assembly according to clause 221, wherein the aerosol provision system further comprises:
a cartridge; and
an aerosol provision device which comprises a cartridge receiving section that includes an interface arranged to cooperatively engage with an interface from the cartridge so as to releasably couple the cartridge to the aerosol provision device.

223. A method of generating an aerosol in an aerosol provision system, wherein the method comprises:
generating a first aerosol using a first aerosol-generating material received from a first reservoir of the aerosol provision system; and
generating a second aerosol using a second aerosol-generating material received from a second reservoir of the aerosol provision system.

224. A method according to clause 223, wherein the first aerosol is generated independently of the second aerosol.

225. A method according to clause 223 or 224, wherein the method further comprises generating the second aerosol using the second aerosol-generating material without heating the second reservoir.

226. A method of generating an aerosol in an aerosol provision system, wherein the method comprises:
generating a first aerosol using a first aerosol-generating material received from a first reservoir of the aerosol provision system; and
delivering an active substance from a second reservoir, to an outlet of the aerosol provision system, for delivering the active substance to a user of the aerosol provision system.

227. A method according to clause 226, wherein the method further comprises:
delivering the active substance to the outlet without heating the second reservoir.

228. A method according to any of clauses 223-227, wherein the method initially comprises:
attaching a consumable comprising the second reservoir to the aerosol provision system before generating the first aerosol.

229. A method according to clause 228, wherein the method further comprises attaching the consumable to an outer surface of the aerosol provision system.

230. A method according to clause 228 or 229, wherein the method further comprises attaching the consumable to a mouthpiece of the aerosol provision system.

231. A method according to any of clauses 228-230, wherein the method further comprises attaching the consumable to a cartridge of the aerosol provision system.

232. A method of retrofitting an aerosol provision system configured to generate a first aerosol using a first aerosol-generating material received from a first reservoir of the aerosol provision system, wherein the method comprises:
releasably coupling a module/consumable, comprising a second reservoir storing an active substance, to the aerosol provision system;
generating a first aerosol using the first aerosol-generating material;
delivering the active substance, from inside the module/consumable, to a user of the aerosol provision system.

233. A method of retrofitting an aerosol provision system configured to generate a first aerosol using a first aerosol-generating material received from a first reservoir of the aerosol provision system, wherein the method comprises:
releasably coupling a consumable, comprising a second reservoir storing an active substance, to the aerosol provision system;
generating a first aerosol using the first aerosol-generating material;
delivering the active substance, from inside the module/consumable, to a user of the aerosol provision system.

234. A method according to clause 233, wherein the method further comprises:
supplying the first aerosol to the module/consumable;
passing the first aerosol through the module/consumable; and
supplying the first aerosol and the active substance to at least one outlet.

235. A method according to clause 226-234, wherein the method further comprises: mixing the first aerosol with the active substance.

236. A method according to any of clauses 226-235, wherein the active substance comprises a flavouring material.

237. A method according to any of clauses 226-236, wherein the active substance comprises a medicament.

238. A method according to any of clauses 226-229, wherein the method further comprises delivering the active substance downstream of the first aerosol.

239. A method according to any of clauses 224-230, wherein the second reservoir is downstream of the first reservoir.

240. A method according to any of clauses 224-231, wherein the method further comprises generating the second aerosol in parallel to the first aerosol.

241. A method according to any of clauses 223-232, wherein the method further comprises supplying the first aerosol through the second reservoir.

242. A module/consumable, for use in an aerosol provision system which is configured to generate a first aerosol using a first aerosol-generating material, wherein the module/consumable is configured to be releasably coupled to the aerosol provision system, and wherein the module/consumable comprises:
a second reservoir for storing a second aerosol-generating material; and
an attachment portion for releasably coupling the module/consumable to the aerosol provision system;
wherein the module/consumable is configured for receiving a first aerosol from the aerosol provision system when the module/consumable is coupled to the aerosol provision system using the attachment portion, and is configured to generate a second aerosol using the second aerosol-generating material when the module/consumable is receiving the first aerosol.

243. A module/consumable, for use in an aerosol provision system which is configured to generate a first aerosol using a first aerosol-generating material, wherein the module/consumable is configured to be releasably coupled to the aerosol provision system, and wherein the module/consumable comprises:
a second reservoir for storing an active substance; and
an attachment portion for releasably coupling the module/consumable to the aerosol provision system;
wherein the module/consumable is configured for receiving a first aerosol from the aerosol provision system when the module/consumable is coupled to the aerosol provision system using the attachment portion, and is configured to deliver the active substance, from the module/consumable, to a user of the aerosol provision system.

244. A module/consumable according to clause 242 or 243, wherein the second aerosol-generating material, or the active substance, comprises a flavouring material.

245. A module/consumable according to clause any of clauses 242-244, wherein the second reservoir is annular.

246. A module/consumable according to any of clauses 242-245, wherein the module further comprises an outlet tube for receiving the first aerosol, wherein the second reservoir at least partially surrounds the outlet tube.

247. A module/consumable according to any of clauses 242-246, wherein the second reservoir comprises a volumetric capacity of no more than 50 ml.

248. A module/consumable according to any of clauses 242-247, wherein the second reservoir comprises a volumetric capacity of no more than 25 ml.

249. A module/consumable according to any of clauses 242-248, wherein the module is disposable.

250. A module according to any of clauses 242-249, wherein the module is configured to be consumable.

251. An assembly comprising the module/consumable according to any of clauses 242-250, and the aerosol provision system to which the module/consumable is configured to be releasably coupled.

252. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing an active substance;
wherein the aerosol provision system comprises an outlet for delivering the active substance from the second reservoir to a user of the aerosol provision system; and
wherein the second reservoir does not comprise a vaporiser or a propellant.

253. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing an active substance;
wherein the aerosol provision system comprises an outlet for delivering the active substance from the second reservoir to a user of the aerosol provision system;
wherein the second reservoir does not comprise a vaporiser or a propellant;
wherein the first aerosol-generating material comprises nicotine and a carrier constituent selected from a combination of glycerine and propylene glycol;
wherein the active substance comprises one or more flavouring material(s) and/or one or more sensate(s).

254. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing an active substance;
wherein the aerosol provision system comprises an outlet for delivering the active substance as a gas phase active substance from the second reservoir to a user of the aerosol provision system; and
wherein the second reservoir does not comprise a vaporiser or a propellant.

255. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing an active substance;
wherein the aerosol provision system comprises an outlet for delivering the active substance as a gas phase active substance from the second reservoir to a user of the aerosol provision system;
wherein the second reservoir does not comprise a vaporiser or a propellant;
wherein the first aerosol-generating material comprises nicotine and a carrier constituent selected from a combination of glycerine and propylene glycol;
wherein the active substance comprises one or more flavouring material(s) and/or one or more sensate(s).

256. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir comprising a porous material for storing an active substance;
wherein the aerosol provision system comprises an outlet for delivering the active substance as a gas phase active substance from the second reservoir to a user of the aerosol provision system; and
wherein the second reservoir does not comprise a vaporiser or a propellant.

257. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir comprising a porous material for storing an active substance;
wherein the aerosol provision system comprises an outlet for delivering the active substance as a gas phase active substance from the second reservoir to a user of the aerosol provision system;
wherein the second reservoir does not comprise a vaporiser or a propellant;
wherein the first aerosol-generating material comprises nicotine and a carrier constituent selected from a combination of glycerine and propylene glycol;
wherein the active substance comprises one or more flavouring material(s) and/or one or more sensate(s).

### First Set of Clauses

1. A module, for use with an aerosol provision system which is configured to generate a first aerosol using a first aerosol-generating material, wherein the module is configured to be releasably coupled to the aerosol provision system, and wherein the module comprises:
a second reservoir for storing a second aerosol-generating material; and
an attachment portion for releasably coupling the module to the aerosol provision system;
wherein the module is configured for receiving a first aerosol from the aerosol provision system when the module is coupled to the aerosol provision system using the attachment portion, and is configured to generate a second aerosol using the second aerosol-generating material when the module is receiving the first aerosol.

2. A module according to clause 1, wherein the module further comprises an outlet tube for receiving the first aerosol, wherein the second reservoir at least partially surrounds the outlet tube.

3. A module according to any preceding clause, wherein the second reservoir is annular.

4. A module according to any preceding clause, wherein the module is configured to selectively prevent generation of the second aerosol from the second aerosol generating material.

5. A module according to any preceding clause, wherein the module is configured to vary the rate at which second aerosol is generated from the second aerosol generating material.

6. A module according to any preceding clause, wherein the module further comprises a throttling portion for varying at least one of:
i) the rate at which air is supplied to the second reservoir;
ii) the rate of generation of the second aerosol; and
iii) the rate at which the second aerosol is configured to flow, or exit through, the module.

7. A module according to clause 6, wherein the throttling portion comprises a mechanical throttling portion.

8. A module according to clause 6 or 7, wherein the throttling portion comprises a mechanical throttling portion.

9. A module according to any of clauses 6-8, wherein the throttling portion is user adjustable.

10. A module according to any of clauses 6-9, wherein the throttling portion comprises a variable sized orifice.

11. A module according to any preceding clause, further comprising an air inlet for supplying air to the second reservoir.

12 A module according to any of clause 11, when further dependent on clause 10, wherein the variable sized orifice comprises the air inlet.

13. A module according to any preceding clause, wherein the second aerosol-generating material comprises a flavouring material.

14. A module according to any preceding clause, wherein the second aerosol-generating material comprises a medicament.

15. A module according to any preceding clause, wherein the second reservoir comprises a volumetric capacity of no more than 50 ml.

16. A module according to any preceding clause, wherein the second reservoir comprises a volumetric capacity of no more than 25 ml.

17. A module according to any preceding clause, wherein the module defines a recess for accommodating the aerosol provision system.

18. A module according to any preceding clause, wherein the module defines a recess for releasably accommodating a mouthpiece of the aerosol provision system.

19. A module according to any preceding clause, wherein the attachment portion is resilient.

20. A module according to any preceding clause, wherein the attachment portion comprises a projection which is configured to engage with, or comprises a recess which is configured to be engaged by, the aerosol provision system for releasably coupling the module to the aerosol provision system.

21. A module according to any preceding clause, wherein the attachment portion comprises a threaded arrangement which is configured to engage with a corresponding threaded arrangement from the aerosol provision system for releasably coupling the module to the aerosol provision system.

22. A module according to any preceding clause, wherein the module is disposable.

23. A module according to any preceding clause, wherein the module is configured to be consumable.

24. An assembly comprising the module according to any preceding clause, and the aerosol provision system to which the module is configured to be releasably coupled.

25. An assembly according to clause 24, wherein the aerosol provision system further comprises a consumable and an aerosol provision device, wherein the aerosol provision device comprises a consumable receiving section that includes an interface arranged to cooperatively engage with an interface from the consumable so as to releasably couple the consumable to the aerosol provision device.

26. An assembly according to clause 25, wherein the module is configured for releasably coupling to the consumable from the aerosol provision system.

27. An assembly according to clause 25 or 26, wherein the module is configured to releasably couple over, or to, a mouthpiece from the consumable for releasably coupling the module to the aerosol provision system.

28. A method of generating an aerosol in an aerosol provision system, wherein the method comprises:
generating a first aerosol using a first aerosol-generating material received from a first reservoir of the aerosol provision system;
generating a second aerosol using a second aerosol-generating material received from a second reservoir from a module which is releasably coupled to the aerosol provision system;
wherein the first aerosol is generated independently of the second aerosol.

29. A method of retrofitting an aerosol provision system configured to generate a first aerosol using a first aerosol-generating material received from a first reservoir of the aerosol provision system, wherein the method comprises:
releasably coupling a module, comprising a second reservoir storing a second aerosol-generating material, to the aerosol provision system;
generating a first aerosol using the first aerosol-generating material;
generating a second aerosol, inside the module, using the second aerosol-generating material.

30. A method according to clause 28 or 29, wherein method further comprises: releasably coupling the module to a consumable from the aerosol provision system.

31. A method according to any of clauses 28-30, wherein method further comprises: releasably coupling the module to a mouthpiece from the aerosol provision system.

32. A method according to any of clauses 28-31, wherein the method further comprises:
supplying the first aerosol to the module;
passing the first aerosol through the module; and
supplying the first aerosol and the second aerosol to at least one outlet from the module.

33. A method according to any of clauses 28-32, wherein the method further comprises: mixing the first aerosol and the second aerosol inside the module.

34. A method according to any of clauses 28-33, wherein the second aerosol-generating material comprises a flavouring material.

35. A method according to any of clauses 28-34, wherein the method further comprises generating the second aerosol downstream of the first aerosol.

36. A method according to any of clauses 28-35, wherein the second reservoir is downstream of the first reservoir.

37. A method according to any of clauses 28-36, wherein the method further comprises supplying the first aerosol through the second reservoir.

38. A method according to any of clauses 28-37, wherein the method further comprises: supplying one or more acids to the first reservoir whilst the first aerosol is being generated.

### Second Set of Clauses

1. A module, for use with a container configured to deliver a fluid through a fluid outlet channel of the container, wherein the module is configured to be releasably coupled to the container, and wherein the module comprises:
a reservoir for storing an aerosol-generating material; and
an attachment portion for releasably coupling the module to the container;
wherein the module is configured for receiving a fluid from the container when the module is coupled to the container using the attachment portion, and is configured to generate an aerosol using the aerosol-generating material when the module is receiving the fluid.

2. A module according to clause 1, wherein the module further comprises an outlet tube for receiving the fluid from the fluid outlet channel of the container, wherein the reservoir at least partially surrounds the outlet tube.

3. A module according to any preceding clause, wherein the reservoir is annular.

4. A module according to any preceding clause, wherein the module is configured to selectively prevent generation of the aerosol from the aerosol generating material.

5. A module according to any preceding clause, wherein the module is configured to vary the rate at which aerosol is generated from the aerosol generating material.

6. A module according to any preceding clause, wherein the module further comprises a throttling portion for varying at least one of:
i) the rate at which air is supplied to the reservoir;
ii) the rate of generation of the aerosol; and
iii) the rate at which the aerosol is configured to flow, or exit through, the module.

7. A module according to clause 6, wherein the throttling portion comprises a mechanical throttling portion.

8. A module according to any of clauses 6-7, wherein the throttling portion is user adjustable.

9. A module according to any of clauses 6-8, wherein the throttling portion comprises a variable sized orifice.

10. A module according to any preceding clause, further comprising a mouthpiece.

11. A module according to any preceding clause, further comprising an air inlet for supplying air to the reservoir.

12 A module according to any of clause 11, when further dependent on clause 9, wherein the variable sized orifice comprises the air inlet.

13. A module according to any preceding clause, wherein the aerosol-generating material comprises a flavouring material.

14. A module according to any preceding clause, wherein the aerosol-generating material comprises a medicament.

15. A module according to any preceding clause, wherein the reservoir comprises a volumetric capacity of no more than 50 ml.

16. A module according to any preceding clause, wherein the reservoir comprises a volumetric capacity of no more than 25 ml.

17. A module according to any preceding clause, wherein the module defines a recess for accommodating at least a portion of the container.

18. A module according to any preceding clause, wherein the module defines a recess for releasably accommodating a lid of the container.

19. A module according to any preceding clause, wherein the attachment portion is resilient.

20. A module according to any preceding clause, wherein the attachment portion comprises a projection which is configured to engage with, or comprises a recess which is configured to be engaged by, the aerosol provision system for releasably coupling the module to the container.

21. A module according to any preceding clause, wherein the attachment portion comprises a threaded arrangement which is configured to engage with a corresponding threaded arrangement from the container for releasably coupling the module to the container.

22. A module according to any preceding clause, wherein the module is disposable.

23. A module according to any preceding clause, wherein the module is configured to be consumable.

24. An assembly comprising the module according to any preceding clause, and the container to which the module is configured to be releasably coupled.

25. An assembly according to clause 24, wherein the module is configured for releasably coupling to a lid from the container.

26. An assembly according to clause 24 or 25, wherein the module is configured to releasably couple over, or to, a mouthpiece from the container for releasably coupling the module to the container.

27. An assembly according to any of clauses 24-26, wherein the container comprises a drinks bottle.

28. A method of supplying an aerosol to a fluid received from a container, wherein the method comprises:
passing a fluid from the container into a module;
generating an aerosol using aerosol-generating material received from a reservoir from the module which is releasably coupled to the container;
wherein the aerosol is generated at the same time as the fluid is passed from the container into the module.

29. A method according to clause 28, wherein the method further comprises:
releasably coupling the module to the container.

30. A method according to clause 28 or 29, wherein the method further comprises:
releasably coupling the module to a lid from the container.

31. A method according to any of clauses 28-30, wherein the method further comprises:
supplying the fluid and the aerosol to at least one outlet from the module.

32. A method according to any of clauses 28-31, wherein the method further comprises:
mixing the fluid and the aerosol inside the module.

33. A method according to any of clauses 28-32, wherein the aerosol-generating material comprises a flavouring material.

34. A method according to any of clauses 28-33, wherein the method further comprises:
passing the fluid from a fluid outlet channel of the container into the module, wherein the reservoir is downstream of the fluid outlet channel.

35. A method according to any of clauses 28-34, wherein the method further comprises supplying the fluid through the reservoir.

### Third Set of Clauses

1. An aerosol provision system for generating an aerosol, wherein the aerosol provision system comprises:
a first reservoir for storing a first aerosol-generating material, wherein the aerosol provision system is configured to generate a first aerosol using the first aerosol-generating material; and
a second reservoir for storing a second aerosol-generating material, wherein the aerosol provision system is configured to generate a second aerosol using the primary second aerosol-generating material.

2. An aerosol provision system according to clause 1, wherein the second reservoir comprises a primary second reservoir, the second aerosol-generating material comprises a primary second aerosol-generating material, and the second aerosol comprises a primary second aerosol;
wherein the aerosol provision system further comprises a secondary second reservoir for storing a secondary second aerosol-generating material, wherein the aerosol provision system is configured to generate a secondary second aerosol using the secondary second aerosol-generating material.

3. An aerosol provision system according to clause 2, wherein the composition of the primary second aerosol-generating material is different to the composition of the secondary second aerosol-generating material.

4. An aerosol provision system according to clause 2 or 3, wherein the primary second aerosol-generating material comprises a flavouring material which is absent from the secondary second aerosol-generating material.

5. An aerosol provision system according to any of clauses 2-4, wherein the aerosol provision system is configured to selectively toggle between generating the primary second aerosol-generating material and the secondary second aerosol-generating material.

6. An aerosol provision system according to any of clauses 2-5, wherein the aerosol provision system is configured to selectively prevent one of the primary and the secondary second aerosols from being generated at the same time as the other of the primary and the secondary second aerosols.

7. An aerosol provision system according to any preceding clause, further comprising a third reservoir for storing a third aerosol-generating material, wherein the aerosol provision system is configured to generate a third aerosol using the third aerosol-generating material.

8. An aerosol provision system according to clause 7, wherein the third aerosol-generating material comprises one or more acids.

9. An aerosol provision system according to clause 7 or 8, wherein the third aerosol is configured to be delivered to the first reservoir.

10. An aerosol provision system according to any preceding clause, wherein the second aerosol-generating material comprises one or more acids.

11. An aerosol provision system according to any preceding clause, wherein the second aerosol is configured to be delivered to the first reservoir.

12. An aerosol provision system according to any of clauses 1-10, wherein the first aerosol is configured to be delivered to the second reservoir.

13. An aerosol provision system according to any preceding clause, further comprising an aerosol generator for generating the first aerosol.

14. An aerosol provision system according to clause 13, wherein the aerosol generator comprises a heating element.

15. An aerosol provision system according to any preceding clause, wherein each second reservoir comprises a substrate for accommodating the aerosol generating material inside the second reservoir.

16. An aerosol provision system according to any preceding clause, further comprising the second aerosol generating material, wherein the second reservoir comprises a headspace.

17. An aerosol provision system according to clause 16, wherein the headspace is no smaller than 10% of the total volumetric capacity of the second reservoir.

18. An aerosol provision system according to any preceding clause, wherein the first reservoir comprises a volumetric capacity of no more than 50 ml.

19. An aerosol provision system according to any preceding clause, wherein the second reservoir comprises a volumetric capacity of no more than 50 ml.

20. An aerosol provision system according to any preceding clause, wherein the aerosol provision system is configured to generate the second aerosol downstream of the first aerosol.

21. A consumable for use in an aerosol provision system for generating an aerosol, wherein the consumable comprises:
a first reservoir for storing a first aerosol-generating material, wherein the aerosol provision system is configured to generate a first aerosol using the first aerosol-generating material; and
a second reservoir for storing a second aerosol-generating material, wherein the aerosol provision system is configured to generate a second aerosol using the primary second aerosol-generating material.

22. An aerosol provision system comprising a consumable according to clause 21 and an aerosol provision device, wherein the aerosol provision device comprises a consumable receiving section that includes an interface arranged to cooperatively engage with an interface from the consumable so as to releasably couple the consumable to the aerosol provision device.

23. A method of generating an aerosol in an aerosol provision system, wherein the method comprises:
generating a first aerosol using a first aerosol-generating material received from a first reservoir of the aerosol provision system; and
generating a second aerosol using a second aerosol-generating material received from a second reservoir of the aerosol provision system.

24. A method according to clause 24, wherein the method further comprises:
supplying one or more acids to the first reservoir whilst the first aerosol is being generated.

25. A method according to clause 23 or 24, wherein the method further comprises:
supplying the first aerosol and the second aerosol to at least one outlet from the aerosol provision system.

26. A method according to clause 25, wherein the at least one outlet from the aerosol provision system comprises at least one mouthpiece outlet from the aerosol provision system.

27. A method according to any of clauses 23-26, wherein the second reservoir comprises a primary second reservoir, the second aerosol-generating material comprises a primary second aerosol-generating material, and the second aerosol comprises a primary second aerosol;
wherein the method further comprises:
stopping generation of the primary second aerosol; and
generating a secondary second aerosol using a secondary second aerosol-generating material received from a secondary second reservoir of the aerosol provision system, wherein the secondary second reservoir is separate from the primary second reservoir.

28. A method according to clause 27, wherein the composition of the primary second aerosol-generating material is different to the composition of the secondary second aerosol-generating material.

29. An method according to clause 27 or 28, wherein the primary second aerosol-generating material comprises a flavouring material which is absent from the secondary second aerosol-generating material.

### Fourth Set of Clauses

1. An aerosol provision system for generating an aerosol, wherein the aerosol provision system comprises:
a first reservoir for storing a first aerosol-generating material, wherein the aerosol provision system is configured to generate a first aerosol using the first aerosol-generating material;
a second reservoir for storing a second aerosol-generating material, wherein the aerosol provision system is configured to generate a second aerosol using the second aerosol-generating material;
wherein the aerosol provision system is configured to generate the first aerosol independently of the second aerosol.

2. An aerosol provision system according to clause 1, further comprising an aerosol generator for generating the first aerosol.

3. An aerosol provision system according to clause 2, wherein the aerosol generator comprises a heating element.

4. An aerosol provision system according to any preceding clause, wherein the aerosol provision system is configured to generate the second aerosol downstream of the first aerosol.

5. An aerosol provision system according to any preceding clause, wherein the aerosol provision system is configured to supply the first aerosol through the second reservoir.

6. An aerosol provision system according to any preceding clause, further comprising an aerosol outlet channel for receiving the first aerosol.

7. An aerosol provision system according to clause 6, wherein the aerosol provision system is configured to supply the second aerosol into the aerosol outlet channel.

8. An aerosol provision system according to clause 7, wherein the aerosol provision system is configured to supply the second aerosol into the aerosol outlet channel via at least one opening in the aerosol outlet channel, wherein the at least one opening comprises a plurality of openings.

9. An aerosol provision system according to any of clauses 6-8, wherein the aerosol outlet channel is configured to supply the first aerosol to a first aerosol outlet from the aerosol provision system, wherein the aerosol provision system is configured to supply the second aerosol into the aerosol outlet channel in a direction which extends towards the first aerosol outlet.

10. An aerosol provision system according to any preceding clause, wherein the aerosol provision system is configured to generate the first aerosol using air supplied from a first air inlet from the aerosol provision system.

11. An aerosol provision system according to clause 10, wherein the aerosol provision system is configured to generate the second aerosol using air supplied from a second air inlet from the aerosol provision system, wherein the first air inlet is separate from the second air inlet.

12. An aerosol provision system according to any preceding clause, wherein the second aerosol-generating material comprises nicotine and/or a flavouring material.

13. An aerosol provision system according to clause 12, wherein the flavouring material comprises at least one of menthol; and/or tobacco.

14. An aerosol provision system according to any preceding clause, wherein the aerosol provision system is configured to generate the first aerosol simultaneously to generating the second aerosol.

15. An aerosol provision system according to any preceding clause, wherein the aerosol provision system is configured to mix the second aerosol with the first aerosol.

16. An aerosol provision system according to any preceding clause, wherein the second reservoir comprises a volumetric capacity of no more than 50 ml.

17. An aerosol provision system according to any preceding clause, wherein the aerosol provision system is configured to:
i) selectively prevent generation of the second aerosol from the second aerosol generating material; and/or
ii) vary the rate at which second aerosol is generated from the second aerosol generating material.

18. An aerosol provision system according to any preceding clause, wherein the aerosol provision system further comprises a throttling portion for varying at least one of:
i) the rate at which air is supplied to the second reservoir;
ii) the rate of generation of the second aerosol; and/or
iii) the rate at which the second aerosol is configured to exit the aerosol provision system.

19. An aerosol provision system according to clause 18, wherein the throttling portion is configured to control a flow rate of air which is delivered to the second reservoir.

20. An aerosol provision system according to any of clauses 18-19, wherein the throttling portion is user adjustable.

21. An aerosol provision system according to any preceding clause, wherein the aerosol provision system further comprises a consumable and an aerosol provision device, wherein the aerosol provision device comprises a consumable receiving section that includes an interface arranged to cooperatively engage with an interface from the consumable so as to releasably couple the consumable to the aerosol provision device, wherein the consumable comprises the first reservoir.

22. A consumable for use in an aerosol provision system for generating an aerosol, wherein the consumable comprises:
a first reservoir for storing a first aerosol-generating material, wherein the aerosol provision system is configured to generate a first aerosol using the first aerosol-generating material;
a second reservoir for storing a second aerosol-generating material, wherein the aerosol provision system is configured to generate a second aerosol using the second aerosol-generating material;
wherein the aerosol provision system is configured to generate the first aerosol independently of the second aerosol.

23. An aerosol provision system comprising a consumable according to clause 22 and an aerosol provision device, wherein the aerosol provision device comprises a consumable receiving section that includes an interface arranged to cooperatively engage with an interface from the consumable so as to releasably couple the consumable to the aerosol provision device.

24. A method of generating an aerosol in an aerosol provision system, wherein the method comprises:
generating a first aerosol using a first aerosol-generating material received from a first reservoir of the aerosol provision system;
generating a second aerosol using a second aerosol-generating material received from a second reservoir of the aerosol provision system;
wherein the first aerosol is generated independently of the second aerosol.

25. A method according to clause 24, wherein the method further comprises:
supplying the first aerosol to an aerosol outlet channel;
supplying the second aerosol to the aerosol outlet channel; and
supplying the first aerosol and the second aerosol to at least one outlet from the aerosol provision system.

26. A method according to clause 24 wherein the method further comprises:
supplying the first aerosol to the second reservoir;
passing the first aerosol through the second reservoir; and
supplying the first aerosol and the second aerosol to at least one outlet from the aerosol provision system.

27. A method according to any of clauses 24-26, wherein the method further comprises: mixing the first aerosol and the second aerosol.

28. A method according to any of clauses 24-27, wherein the second aerosol-generating material comprises a flavouring material.

29. A method according to any of clauses 24-28, wherein the method further comprises generating the second aerosol downstream of the first aerosol.

30. A method according to any of clauses 24-29, wherein the method further comprises: supplying one or more acids to the first reservoir whilst the first aerosol is being generated.

1. An aerosol provision system for generating an aerosol, wherein the aerosol provision system comprises:
a first reservoir for storing a first aerosol-generating material, wherein the aerosol provision system is configured to generate a first aerosol using the first aerosol-generating material; and
a second reservoir for storing an active substance, wherein the aerosol provision system comprises an outlet for delivering the active substance from the second reservoir to a user of the aerosol provision system.

2. An aerosol provision system according to claim 1, wherein the first reservoir is separate from the second reservoir.

3. An aerosol provision system according to claim 1, wherein the active substance comprises a second aerosol-generating material, wherein the aerosol provision system is configured to generate a second aerosol using the second aerosol-generating material.

4. An aerosol provision system according to claim 3, wherein the aerosol provision system is configured to generate the first aerosol independently of the second aerosol.

5. An aerosol provision system according to claim 3, wherein the aerosol provision system is configured to generate the second aerosol without actively heating the second aerosol generating material.

6. An aerosol provision system according to claim 1, wherein the aerosol provision system is configured to deliver the active substance downstream of the first aerosol.

7. An aerosol provision system according to claim 1, wherein the second reservoir is downstream of the first reservoir.

8. An aerosol provision system according to claim 1, further comprising an aerosol outlet channel for receiving the first aerosol, wherein the aerosol provision system is configured to supply the active substance into the aerosol outlet channel.

9. An aerosol provision system according to claim 1:
wherein the aerosol provision system is configured to generate the first aerosol using air supplied from a first air inlet from the aerosol provision system;
wherein the aerosol provision system is configured to supply air to the active substance using air supplied from a second air inlet from the aerosol provision system;
wherein the first air inlet is separate from the second air inlet.

10. An aerosol provision system according to claim 1, further comprising a mouthpiece, wherein the second reservoir is configured to engage the mouthpiece.

11. An aerosol provision system according to claim 10, wherein the second reservoir is configured to engage an outer surface of the mouthpiece.

12. An aerosol provision system according to claim 10, wherein the second reservoir is configured to operate as a sleeve which can engage around the mouthpiece.

13. An aerosol provision system according to claim 10, wherein the second reservoir is configured to be located within a recess of the mouthpiece, wherein the recess is shaped to accommodate at least a portion of the second reservoir.

14. An aerosol provision system according to claim 1, wherein the second reservoir comprises a compressible, permeable and/or penetrable portion.

15. An aerosol provision system according to claim 1, wherein the second reservoir comprises a compressible and/or penetrable portion, wherein the compressible and/or penetrable portion is configured such that compression and/or penetration of the compressible and/or penetrable portion is configured to allow the active substance to be released from the second reservoir.

16. An aerosol provision system according to claim 1, wherein the second reservoir comprises a permeable portion, wherein the permeable portion is configured to allow the active substance to be selectively released from the second reservoir as air is supplied to the active substance from a second air inlet from the aerosol provision system.

17. An aerosol provision system according to claim 1, wherein the second reservoir comprises an attachment means for releasably attaching the second reservoir to a portion of the aerosol provision system.

18. An aerosol provision system according to claim 17, when further dependent on claim 10 at least, wherein the portion comprises the mouthpiece.

19. An aerosol provision system according to claim 17, when further dependent on claim 10 at least, wherein the portion comprises an aerosol outlet tube, from the mouthpiece, which is configured to receive the first aerosol.

20. An aerosol provision system according to claim 17, wherein the attachment means comprises an adhesive.

21. An aerosol provision system according to claim 1, further comprising the active substance in the second reservoir.

22. An aerosol provision system according to claim 1, wherein the active substance comprises:
a liquid and/or a gel phase active substance,
a gas phase active substance,
a solid phase active substance,
one or more flavouring material(s),
nicotine,
one or more cannabinoid(s),
tetrahydrocannabinol and/or cannabidiol,
one or more sensate(s), or
one or more cooling agents and/or warming agents.

23-30. (canceled)

31. An aerosol provision system according to claim 1, wherein the second reservoir further comprises a carrier constituent for the active substance, wherein the carrier constituent optionally comprises a liquid; preferably the liquid is selected from: water, glycerine, propylene glycol, and combinations thereof.

32. An aerosol provision system according to claim 1, wherein the second reservoir is annular.

33. An aerosol provision system according to claim 1, wherein the second reservoir comprises a volumetric capacity of no more than 25 ml.

34. An aerosol provision system according to claim 10, wherein the aerosol provision system further comprises a consumable which comprises the second reservoir.

35. An aerosol provision system according to claim 34, wherein the consumable is configured to releasably couple over, or to, the mouthpiece from the aerosol provision system.

36. An aerosol provision system according to claim 1, wherein the second reservoir comprises paper, wherein the paper is configured for storing the active substance.

37. An aerosol provision system according to claim 1, wherein the second reservoir comprises cardboard, wherein the cardboard is configured for storing the active substance.

38. A consumable, for use in an aerosol provision system for generating a first aerosol, wherein the consumable comprises a reservoir for storing an active substance, wherein the consumable comprises an outlet for delivering the active substance from the reservoir to a user of the aerosol provision system.

39. A consumable according to claim 38, wherein the consumable is configured to deliver the active substance from the second reservoir, via the outlet, without heating the active substance.

40. A consumable according to claim 38, wherein the reservoir comprises a compressible and/or penetrable portion, wherein the compressible and/or penetrable portion is configured such that compression and/or penetration of the compressible and/or penetrable portion is configured to allow the active substance to be released from the reservoir to the outlet.

41. A consumable according to claim 38, wherein the reservoir comprises a permeable portion, wherein the permeable portion is configured to allow the active substance to be selectively released from the reservoir as air is supplied to the active substance from a second air inlet from the aerosol provision system.

42. A consumable according to claim 38, wherein the consumable comprises an attachment means for releasably attaching the consumable to a portion of the aerosol provision system.

43. A consumable according to claim 42, wherein the attachment means comprises an adhesive.

44. A consumable according to claim 38, wherein the reservoir comprises a resilient outer shell for storing the active substance.

45. A consumable according to claim 38, further comprising the active substance in the reservoir.

46. A consumable according to claim 38, wherein the active substance comprises any combination of:
i) a flavouring material;
ii) nicotine;
iii) one or more cannabinoids, preferably tetrahydrocannabinol and/or cannabidiol;
iv) a sensate, preferably a cooling agent and/or a warming agent.

47. A consumable according to claim 38, wherein the reservoir further comprises a carrier constituent for the active substance, wherein the carrier constituent optionally comprises a liquid; preferably the liquid is selected from: water, glycerine, propylene glycol, and combinations thereof.

48. An assembly comprising a consumable according to claim 38 and the aerosol provision system.

49. An assembly according to claim 48, wherein the aerosol provision system further comprises:
a cartridge; and
an aerosol provision device which comprises a cartridge receiving section that includes an interface arranged to cooperatively engage with an interface from the cartridge so as to releasably couple the cartridge to the aerosol provision device.

50. A method of generating an aerosol in an aerosol provision system, wherein the method comprises:
generating a first aerosol using a first aerosol-generating material received from a first reservoir of the aerosol provision system; and
delivering an active substance from a second reservoir, to an outlet of the aerosol provision system, for delivering the active substance to a user of the aerosol provision system.

51. A method according to claim 50, wherein the method further comprises:
delivering the active substance to the outlet without heating the second reservoir.

52. A method according to claim 50, wherein the method initially comprises:
attaching a consumable comprising the second reservoir to the aerosol provision system, before generating the first aerosol.

53. A method according to claim 52, wherein the method further comprises:
attaching the consumable to an outer surface of the aerosol provision system.

54. A method according to claim 52, wherein the method further comprises:
attaching the consumable to a mouthpiece of the aerosol provision system.

55. A method according to claim 52, wherein the method further comprises:
attaching the consumable to a cartridge of the aerosol provision system.

56. A method of retrofitting an aerosol provision system configured to generate a first aerosol using a first aerosol-generating material received from a first reservoir of the aerosol provision system, wherein the method comprises:
releasably coupling a consumable comprising a second reservoir storing an active substance, to the aerosol provision system;
generating a first aerosol using the first aerosol-generating material;
delivering an active substance from the second reservoir, to an outlet of the consumable or the aerosol provision system, for delivering the active substance to a user of the aerosol provision system.

57. A method according to claim 50, wherein the active substance comprises any combination of:
i) a flavouring material;
ii) nicotine;
iii) one or more cannabinoids, preferably tetrahydrocannabinol and/or cannabidiol;
iv) a sensate, preferably a cooling agent and/or a warming agent.

58. A method according to claim 50, wherein the method further comprises:
delivering the active substance downstream of the first aerosol.

59. A method according to claim 50, wherein the method further comprises:
delivering the active substance in parallel to the first aerosol.
